(19) United States
(12) Patent Application Publication
(10) Pub. No.: US 2017/0233446 A1
(43) Pub. Date: Aug. 17, 2017

(54) METHODS AND COMPOSITIONS USING KLOTHO-FGF FUSION POLYPEPTIDES

(71) Applicants: David GLASS, Cambridge, MA (US); Shou-Ih HU, New Providence, NJ (US)

(72) Inventors: David GLASS, Cambridge, MA (US); Shou-Ih HU, New Providence, NJ (US)

(73) Assignee: Novartis AG, Basel (CH)

(21) Appl. No.: 15/271,198

(22) Filed: Sep. 20, 2016

Related U.S. Application Data
(63) Continuation-in-part of application No. 14/531,620, filed on Nov. 3, 2014, now Pat. No. 9,475,857.
Application No. 14/531,620 is a division of application No. 13/796,711, filed on Mar. 12, 2013, now Pat. No. 8,932,589.
Application No. 13/796,711 is a continuation-in-part of application No. 12/360,970, filed on Jan. 28, 2009, now Pat. No. 8,481,031.
(60) Provisional application No. 61/063,015, filed on Jan. 28, 2008.

(51) Int. Cl. C07K 14/50; C12N 9/24

(52) U.S. Cl. CPC C07K 14/50; C12N 9/2402; C12Y 302/01031; C07K 2319/02; C07K 2319/74; A61K 38/00

(57) ABSTRACT

The present invention is directed to methods, kits and compositions for preventing or treating age-related conditions or metabolic disorders. The Klotho fusion polypeptides of the invention include at least a Klotho protein or an active fragment thereof The Klotho fusion proteins are useful in the treatment and prevention of a variety of age-related conditions and metabolic disorders.

This application is a continuation-in-part of U.S. application Ser. No. 14/531,620 filed Nov. 3, 2014, now U.S. Pat. No. 9,475,857, which is a divisional of U.S. application Ser. No. 13/796,711 filed Mar. 12, 2013, now U.S. Pat. No. 8,932,589, which is a continuation-in-part of U.S. application Ser. No. 12/360,970 filed Jan. 28, 2009, now U.S. Pat. No. 8,481,031, which claims priority to U.S. Provisional Application Ser. No. 61/063,015 filed 28 Jan. 2008, the entire-contents of all these applications are incorporated herein by reference in their entirety.

### 1. BACKGROUND

The alpha-Klotho gene encodes a 130 kDa single pass type I transmembrane protein with an extracellular domain and a short cytoplasmic domain. The extracellular domain of alpha-Klotho protein comprises two subdomains termed, KL-D1 and KL-D2. These two subdomains share sequence homology to 13-glucosidase of bacteria and plants. The extracellular domain of the alpha-Klotho protein may be bound to the cell surface by the transmembrane domain or may be cleaved and released into the extracellular milieu. Cleavage of the extracellular domain appears to be facilitated by local low extracellular Ca²⁺ concentrations.

In addition to alpha-Klotho, a homolog of alpha-Klotho, beta-Klotho, has been identified (Ito et al., Mech. Dev. 98:115-9 (2000)). Beta-Klotho is also a single pass type I transmembrane protein with extracellular KL-D1 and KL-D2 subdomains.

Modulation of alpha-Klotho expression has been demonstrated to produce aging related characteristics in mammals. Mice homozygous for a loss of function mutation in the alpha-Klotho gene develop characteristics resembling human aging, including shortened lifespan, skin atrophy, muscle wasting, arteriosclerosis, pulmonary emphysema and osteoporosis (Kuro-o et al., Nature, 390:45-51 (1997)). In contrast, overexpression of the alpha-Klotho gene in mice extends lifespan and increases resistance to oxidative stress relative to wild-type mice (Kurosu et al., Science 309:1829-1833 (2005); Yamamoto et al., J. Biol. Chern. 280:38029-38034 (2005)).

Fibroblast growth factors (FGFs) constitute a family of homologous polypeptide growth factors expressed in many organisms (Omitz and Itoh, Genome Biol. 2: reviews 3005.1-3005.12 (2001)). Among vertebrate species, FGFs are highly conserved in both gene structure and amino-acid sequence, having between 13-71% amino acid identity with one another. In humans, there are 22 known members of the FGF family (FGF15 is the mouse ortholog of human FGF19, hence there is no human FGF15). During early development, FGFs regulate cell proliferation, migration, and differentiation, but in the adult organism, FGFs maintain homeostasis, function in tissue repair, and respond to injury.

FGFs function as growth factors by binding and thereby activating cell-surface FGF receptors. FGF receptors (FGFRs) are tyrosine kinase receptors that activate signal transduction through autophosphorylation of FGFR, phosphorylation of FRS2 (FGF receptor substrate 2) and ERK1/2 (extracellular signal-regulated protein kinase 112), and activating Egr-1 (early growth response-1). FGFs also have a high affinity for heparin sulfate proteoglycans. When bound to FGFs, heparin sulfate enhances the activation of FGFRs.

Recent studies have demonstrated strikingly similar biological characteristics between FGF23-deficient mice and alpha-Klotho-deficient mice (Shimada et al., J. Clin. Invest. 113:561-568 (2004); Yoshida et al. Endocrinology 143:683-689 (2002)), indicating functional crosstalk between FGF23 and alpha-Klotho. These studies led to the identification of alpha-Klotho as an obligatory partner of FGF23, in terms of both binding and signaling through its cognate FGF receptors (Urakawa et al., Nature 22:1524-6 (2007)). The alpha-Klotho gene is mainly expressed in kidney, parathyroid gland and choroid plexus. It is hypothesized that the tissue-specific expression of alpha-Klotho restricts activation of FGF23 signaling to those tissues.

Similar to FGF23/alpha-Klotho, beta-Klotho is an obligatory partner of FGF19 and FGF21, both in binding and in signaling through their respective cognate FGF receptors (Ogawa et al., Proc. Natl. Acad. Sci. USA 104:7432-7 (2007); Lin et al., J. Biol Chern. 282:27227-84 (2007); and Wu et al., J. Biol. Chern. 282:29069-72 (2007)). Such studies have also demonstrated the involvement of beta-Klotho in regulating tissue-specific metabolic activity. Beta-Klotho was initially shown to act with FGF21 as a cofactor for regulating carbohydrate and lipid metabolism in adipose tissue. Beta-Klotho in conjunction with FGF19 regulates bile acid metabolism in liver, thus explaining elevated bile synthesis in beta-Klotho deficient mice (Ito et al., J Clin Invest. 2005 August; 115(8):2202-8).

U.S. Pat. No. 6,579,850 describes polypeptides and compositions comprising an alpha-Klotho polypeptide. Human and mouse alpha-Klotho polypeptides are disclosed. The patent also disclosed that compositions comprising the polypeptides are useful in treating a syndrome resembling premature aging, treating adult diseases, and suppressing aging.

U.S. Pat. No. 7,223,563 describes isolated nucleic acids encoding the FGF23 polypeptide sequence or recombinant cells comprising such an isolated nucleic acid. The patent further relates to methods of diagnosing and treating hypophosphatemic and hyperphosphatemic disorders, osteoporosis, dermatomyositis, and coronary artery disease.

U.S. Pat. No. 7,259,248 describes isolated nucleic acids encoding the FGF21 polypeptide sequence. The patent further relates to methods of diagnosing and treating liver disease, conditions related to thymic function, and methods of treating conditions of the testis.

### 2. SUMMARY OF THE INVENTION

The present invention is directed to methods, kits and compositions for preventing or treating age-related conditions or metabolic disorders with Klotho fusion polypeptides or soluble Klotho polypeptides. The Klotho fusion polypeptides of the present invention are formed of a Klotho protein or an active fragment thereof (e.g., sKlotho). In some embodiments, the present invention provides a Klotho fusion polypeptide comprising a Klotho protein or an active fragment thereof and a fibroblast growth factor or an active fragment thereof

In a first aspect, the invention provides a fusion polypeptide having at least one extracellular subdomain of a Klotho protein and a fibroblast growth factor or an active fragment thereof The Klotho extracellular domain may be derived from either the alpha or beta Klotho isoforms. Further, although the FGF component of the Klotho fusion polypeptide is described primarily with reference to fibroblast growth factor-19, fibroblast growth factor-21 and fibroblast growth factor-23, it is contemplated that any of the twenty-three known FGFs can be used in practicing the invention. The reader of the instant application may assume that each of every combination of alpha or beta extracellular domain with each human FGF protein or an active fragment thereof are individually and specifically contemplated.

According to the present invention, the extracellular domain of the Klotho protein can include one or both of the KL-D1 and KL-D2 domains of a Klotho protein. In some embodiments, the Klotho fusion polypeptide of the invention has at least two extracellular subdomains of a Klotho protein. For example, the two extracellular subdomains can be two KL-D1 domains in tandem repeats, two KL-D2 domains in tandem repeats, or one KL-D1 domain and one KL-D2 domain. In one embodiment, the fusion polypeptide of the invention comprises amino acids 28-292 of the full length alpha Klotho protein. In another embodiment, the fusion polypeptide of the invention comprises amino acids 52-997 of the full length beta Klotho protein.

According to the present invention, a polypeptide comprising at least one extracellular subdomain of a Klotho protein and a FGF or an active fragment thereof may be linked together covalently, for example, chemically linked or fused in frame by a peptide bond. They may also linked via a linker. Non-limiting examples of polypeptide linker are SEQ ID NOs: 11, 12, 13, 14, 15, 16, 17, and 18. Such linkers may comprise at least one and up to about 30 repeats of SEQ ID NOs: 11, 12, 13, 14, 15, 16, 17 and 18.

According to the present invention, the extracellular subdomain of a Klotho protein and the fibroblast growth factor can be operatively linked to one another in a variety of orientations and manners. For example, the extracellular subdomain of the Klotho protein can be operatively linked to the N-terminus of the fibroblast growth factor or alternatively the fibroblast growth factor can be operatively linked to the N-terminus of an extracellular subdomain of the Klotho protein.

In one embodiment, the present invention provides a fusion polypeptide comprising a sKlotho of a Klotho protein and a linker. In another embodiment, the present invention provides a fusion polypeptide comprising a sKlotho of the alpha Klotho protein and a linker. In another embodiment, the present invention provides a fusion polypeptide comprising a sKlotho of the beta Klotho protein and a linker. In yet another embodiment, the present invention provides a human FGF protein or an active fragment thereof (e.g., without signal peptide) and a linker. Pharmaceutical compositions comprising the fusion proteins of the invention and their uses for treating or preventing age-related conditions or metabolic disorders are also encompassed by the present invention.

In one embodiment, the present invention provides a fusion polypeptide comprising a sKlotho of alpha Klotho protein with signal peptide fused (directly or indirectly via a linker) to FGF-23. In another embodiment, the present invention provides a fusion polypeptide comprising a sKlotho of alpha Klotho protein without signal peptide fused (directly or indirectly via a linker) to FGF-23. In another embodiment, the present invention provides sKlotho of alpha Klotho protein with signal peptide fused (directly or indirectly via a linker) to FGF-23 without signal peptide. In another embodiment, the present invention provides a fusion polypeptide comprising sKlotho of alpha Klotho protein without signal peptide fused (directly or indirectly via a linker) to FGF-23 without signal peptide.

In one embodiment, the present invention provides a fusion polypeptide comprising a sKlotho of alpha Klotho protein with signal peptide fused (directly or indirectly via a linker) to FGF-23 (R179Q) variant. In another embodiment, the present invention provides a fusion polypeptide comprising a sKlotho of alpha Klotho protein without signal peptide fused (directly or indirectly via a linker) to FGF-23 (R179Q) variant. In another embodiment, the present invention provides sKlotho of alpha Klotho protein with signal peptide fused (directly or indirectly via a linker) to FGF-23 (R179Q) variant without signal peptide. In another embodiment, the present invention provides a fusion polypeptide comprising sKlotho of alpha Klotho protein without signal peptide fused (directly or indirectly via a linker) to FGF-23 (R179Q) variant without signal peptide.

In one embodiment, the present invention provides a fusion polypeptide comprising (1) sKlotho of alpha Klotho protein with signal peptide; (2) a linker; and (3) FGF-23 (R179Q) variant without signal peptide. In another embodiment, the present invention provides a fusion polypeptide comprising (1) sKlotho of alpha Klotho protein without signal peptide; (2) a linker; and (3) FGF-23 (R179Q) variant without signal peptide. In some embodiments, the fusion polypeptides of the invention are glycosylated.

In one embodiment, the present invention provides a fusion polypeptide comprising (1) sKlotho of alpha Klotho protein with signal peptide (SEQ ID NO: 44 or SEQ ID NO:45); (2) a linker comprising SEQ ID NO:11; and (3) FGF-23 (R179Q) variant without signal peptide (SEQ ID NO: 43). In another embodiment, the present invention provides a fusion polypeptide comprising (1) sKlotho of alpha Klotho protein without signal peptide (SEQ ID NO:7); (2) a linker comprising SEQ ID NO:11; and (3) FGF-23 (R179Q) variant without signal peptide (SEQ ID NO: 43). In one embodiment, the present invention provides a fusion polypeptide comprising the amino acid sequence of SEQ ID NO:19, 20, 40, or 41. In some embodiments, the fusion polypeptides of the invention are glycosylated.

In one embodiment, the present invention provides a fusion polypeptide comprising sKlotho of alpha Klotho protein with signal peptide (SEQ ID NO:44 or SEQ ID NO:45); and a linker comprising SEQ ID NO:11. In another embodiment, the present invention provides a fusion polypeptide comprising sKlotho of alpha Klotho protein without signal peptide (SEQ ID NO:7); and a linker comprising SEQ ID NO:11. In some embodiments, the fusion polypeptides of the invention are glycosylated.

In one embodiment, the present invention provides a fusion polypeptide comprising a human FGF protein or an active fragment thereof (e.g., without the signal peptide); and a linker comprising SEQ ID NO:11. In some embodiments, the fusion polypeptides of the invention are glycosylated.

In one embodiment, the present invention provides a pharmaceutical composition (e.g., in an intra-muscular administering form) comprising (e.g., as a sole pharmaceutically active ingredient) a fusion polypeptide (e.g., glycosylated or non-glycosylated) that comprises (1) sKlotho of alpha Klotho protein with signal peptide (SEQ ID NO: 44 or SEQ ID NO:45); (2) a linker comprising SEQ ID NO:11; and (3) FGF-23 (R179Q) variant without signal peptide (SEQ ID NO: 43); and uses of the pharmaceutical composition for treating and/or preventing age-related conditions, such as muscular atrophy. In another embodiment, the present invention provides a pharmaceutical composition (e.g., in an intra-muscular administering form) comprising (e.g., as a sole pharmaceutically active ingredient) a fusion polypeptide (e.g., glycosylated or non-glycosylated) that comprises (1) sKlotho of alpha Klotho protein without signal peptide (SEQ ID NO:7); (2) a linker comprising SEQ ID NO:11; and (3) FGF-23 (R179Q) variant without signal peptide (SEQ ID NO: 43); and uses of the pharmaceutical composition for treating and/or preventing age-related conditions, such as muscular atrophy. In one embodiment, the present invention provides a pharmaceutical composition (e.g., in an intra-muscular administering form) comprising (e.g., as a sole pharmaceutically active ingredient) a fusion polypeptide (e.g., glycosylated or non-glycosylated) comprising the amino acid sequence of SEQ ID NO:19, 20, 40, or 41; and uses of the pharmaceutical composition for treating and/or preventing age-related conditions, such as muscular atrophy.

In one embodiment, the present invention provides a pharmaceutical composition (e.g., in an intra-muscular administering form) comprising (e.g., as a sole pharmaceutically active ingredient) a fusion polypeptide (e.g., glycosylated or non-glycosylated) that comprises sKlotho of alpha Klotho protein with signal peptide (SEQ ID NO:44 or SEQ ID NO:45); and a linker comprising SEQ ID NO:11; and uses of the pharmaceutical composition for treating and/or preventing age-related conditions, such as muscular atrophy. In another embodiment, the present invention provides a pharmaceutical composition (e.g., in an intra-muscular administering form) comprising (e.g., as a sole pharmaceutically active ingredient) a fusion polypeptide (e.g., glycosylated or non-glycosylated) comprising sKlotho of alpha Klotho protein without signal peptide (SEQ ID NO:7); and a linker comprising SEQ ID NO:11; and uses of the pharmaceutical composition for treating and/or preventing age-related conditions, such as muscular atrophy.

In one embodiment, the present invention provides a pharmaceuticals composition comprising (e.g., as a sole pharmaceutically active ingredient) a fusion polypeptide (e.g., glycosylated or non-glycosylated) that comprises a human FGF protein or an active fragment thereof (e.g., without the signal peptide); and a linker comprising SEQ ID NO: 11.

Pharmaceutical compositions comprising the fusion proteins of the invention and their uses for treating or preventing age-related conditions (e.g., muscle atrophy) or metabolic disorders (e.g., diabetes) are also encompassed by the present invention.

In one embodiment, the present invention provides a fusion polypeptide that is at least 85%, at least 90%, at least 91%, at least 92%, at least 93%, at least 94%, at least 95%, at least 96%, at least 96%, at least 97%, at least 98%, at least 99% identical to SEQ ID NO: 19. In another embodiment, the present invention provides a fusion polypeptide that is at least 85%, at least 90%, at least 91%, at least 92%, at least 93%, at least 94%, at least 95%, at least 96%, at least 96%, at least 97%, at least 98%, at least 99% identical to SEQ ID NO: 20.

In one embodiment, the present invention provides a fusion polypeptide that is at least 85%, at least 90%, at least 91%, at least 92%, at least 93%, at least 94%, at least 95%, at least 96%, at least 96%, at least 97%, at least 98%, at least 99% identical to SEQ ID NO: 40. In another embodiment, the present invention provides a fusion polypeptide that is at least 85%, at least 90%, at least 91%, at least 92%, at least 93%, at least 94%, at least 95%, at least 96%, at least 96%, at least 97%, at least 98%, at least 99% identical to SEQ ID NO: 41.

In one embodiment, the present invention provides a fusion polypeptide comprising a sKlotho of beta Klotho protein with signal peptide fused (directly or indirectly via a linker) to FGF-19 or an active fragment thereof In another embodiment, the present invention provides a fusion polypeptide comprising a sKlotho of beta Klotho protein without signal peptide fused (directly or indirectly via a linker) to FGF-19 or an active fragment thereof In another embodiment, the present invention provides a fusion polypeptide comprising a sKlotho of beta Klotho protein with signal peptide fused (directly or indirectly via a linker) to FGF-21 or an active fragment thereof In another embodiment, the present invention provides a fusion polypeptide comprising a sKlotho of beta Klotho protein without signal peptide fused (directly or indirectly via a linker) to FGF-21 or an active fragment thereof

The invention provides nucleic acid sequences encoding any of the Klotho fusion polypeptides described herein and host cells containing the nucleic acids.

The invention also provides composition having any of the Klotho fusion polypeptides contemplated herein. The compositions of the invention can further include heparin.

The invention also provides a method for treating or preventing an age-related condition in an individual. An individual (e.g, human) is administered a therapeutically effective dose of a pharmaceutical composition containing a Klotho fusion polypeptide, having at least one extracellular subdomain of a Klotho protein (e.g., alpha Klotho protein) and a fibroblast growth factor or an active fragment thereof so as to treat or prevent the age-related condition. In particular, the invention provides a method of treating or preventing muscle wasting comprising administering to an individual (e.g., human) an therapeutically effective amount of a fusion polypeptide having at least one extracellular subdomain of an alpha Klotho protein and a fibroblast growth factor (or an active fragment thereof).

Additionally, the invention provides a method for treating or preventing a metabolic disorder in an individual. An individual is administered a therapeutically effective dose of a pharmaceutical composition containing a fusion polypeptide of the invention, having at least one extracellular subdomain of a Klotho protein and a fibroblast growth factor (or an active fragment thereof) so as to treat the metabolic disorder. In particular, a fusion polypeptide of the invention having at least one extracellular subdomain of a beta-Klotho protein and a fibroblast growth factor 21 is useful for treating a metabolic disorder.

Klotho-FGF23 fusion polypeptides of the invention can be used for treating or preventing hyperphosphatemia or calcinosis in an individual. A pharmacologically effective dose of a pharmaceutical composition containing the Klotho fusion polypeptide of the invention, having at least one extracellular subdomain of a Klotho protein and a fibroblast growth factor, is administered to treat or prevent hyperphosphatemia or calcinosis. In particular, a Klotho fusion polypeptide of the invention having at least one extracellular subdomain of an alpha Klotho protein and a fibroblast growth factor 23 is useful for treating hyperphosphatemia or calcinosis.

Klotho-FGF23 fusion polypeptides of the invention can be used for treating or preventing chronic renal disease or chronic renal failure in an individual. A therapeutically effective dose of a pharmaceutical composition containing the Klotho fusion polypeptide of the invention, having at least one extracellular subdomain of a Klotho protein (e.g., alpha Klotho protein) and a fibroblast growth factor, is administered to treat or prevent chronic renal disease or chronic renal failure.

Klotho-FGF23 fusion polypeptides of the invention can be used for treating or preventing cancer (e.g., breast cancer) in an individual. A therapeutically effective dose of a pharmaceutical composition containing the Klotho fusion polypeptide of the invention, having at least one extracellular subdomain of a Klotho protein (e.g., alpha Klotho protein) and a fibroblast growth factor, is administered to treat or prevent cancer or breast cancer.

The present invention provides fusion polypeptides comprising at least one extracellular subdomain of Klotho protein and a FGF or an active fragment thereof for use in medicine. In one embodiment, the present invention provides fusion polypeptides comprising at least one extracellular subdomain of Klotho protein and a FGF or an active fragment thereof for use in treating or preventing muscle atrophy. The present invention also provides a method of treating or preventing an age related condition (e.g., muscle atrophy) comprising administering to an individual in need thereof a therapeutically effective dose of a pharmaceutical composition comprising a soluble Klotho protein.

The invention also includes kits for treating or preventing an age-related disorder or metabolic disorder in an individual. The kit includes instructions for use and a purified Klotho fusion polypeptide having at least one extracellular subdomain of a Klotho protein and a fibroblast growth factor.

The invention also provides a kit for producing a Klotho fusion polypeptide of the invention. The kit of the invention includes instructions for use and a nucleic acid encoding a Klotho fusion polypeptide, having at least one extracellular subdomain of Klotho protein and a fibroblast growth factor.

### 3. BRIEF DESCRIPTION OF THE DRAWINGS

FIGS. 1A and 1B illustrate several different embodiments of the Klotho fusion polypeptides of the invention. The represented fusion polypeptides include one or more Klotho extracellular subdomains operatively linked to a fibroblast growth factor. Polypeptides containing one or more Klotho extracellular subdomains include, for example, an extracellular domain of

Klotho (e.g., a.a. 1 to 982 of human Klotho), or an active fragment of Klotho.

FIGS. 2A-2Z-F illustrate the amino acid and nucleic acid sequences of several Klotho fusion polypeptides of the invention and components thereof (e.g., Klotho extracellular domain, FGF).

FIGS. 3A-3C depict protein expression of an sKlotho-FGF23 fusion protein. FIG. 3A shows that sKlotho-FGF23 fusion protein was detected in conditioned media by Western blotting with anti-FGF23 antibodies. FIG. 3B shows that sKlotho-FGF23 fusion protein was detected in conditioned media by SDS-PAGE and Coomassie blue staining FIG. 3C shows a highly purified sKlotho-FGF23-6× His fusion protein, analyzed by SDS-PAGE and Coomassie blue staining.

FIG. 4 illustrates the results of an Egr-1 luciferase assay comparing the activation level of Egr-1 in cells treated with conditioned media containing either a Klotho fusion polypeptide, a FGF 23 polypeptide only, a soluble Klotho (sKlotho) polypeptide only, and a soluble Klotho polypeptide in combination with a FGF 23 polypeptide in the absence or presence of heparin (20 1-1 g/ml).

FIGS. 5A-SB depict the results of an Egr-1 luciferase assay comparing the activation level of Egr-1 in cells treated with purified Klotho fusion polypeptide, FGF 23 polypeptide, or soluble Klotho polypeptide in the absence or presence of heparin. Figure SA shows an the results of an experiment comparing the activation level of Egr-1 in cells treated with FGF 23 alone, sKlotho-His (10 nM or 20 nM) and a combination of FGF 23 and sKlotho-His (10 nM or 20 nM) in the absence or presence of heparin (20 1-1 g/ml). Figure SB shows Egr-1 luciferase reporter activity in cells treated with sKlotho-FGF23-His fusion (0 nM, 0.6 nM, 1.21 nM, 2.41 nM, 4.83 nM, 9.65 nM, and 19.3 nM).

FIGS. 6A-6B illustrate the effect of treatment with a purified sKlotho fusion polypeptide on C2C12 muscle cells. FIG. 6A shows measurements of myotube diameter in C2C12 muscle cells treated with either IGF-1 (10 nM), FGF2 (20 nglml), or a purified Klotho fusion polypeptide (20 nM), in the absence or presence of dexamethasone (100 J.LM). FIG. 6B shows the phosphorylation of signaling pathway proteins in C2C12 muscle cells by IGF-1 (10 nM), FGF2 (20 nglml), or a purified Klotho fusion polypeptide (20 nM), in the absence or presence of rapamycin (40 nM).

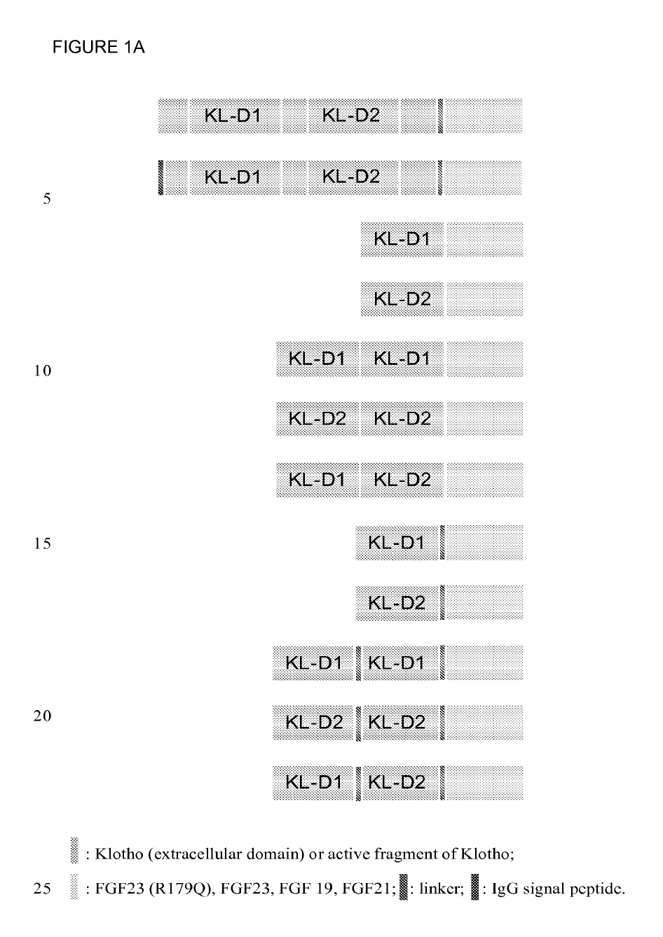

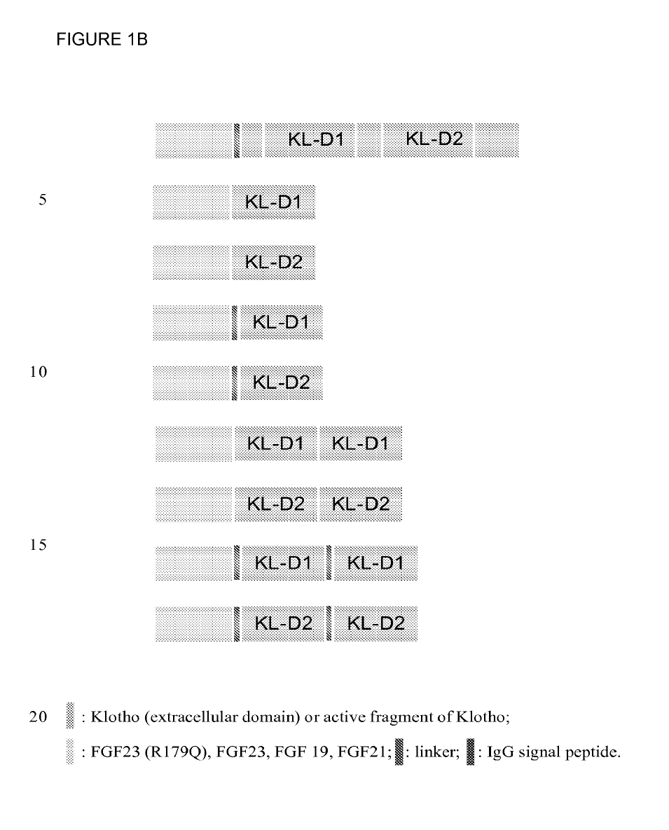

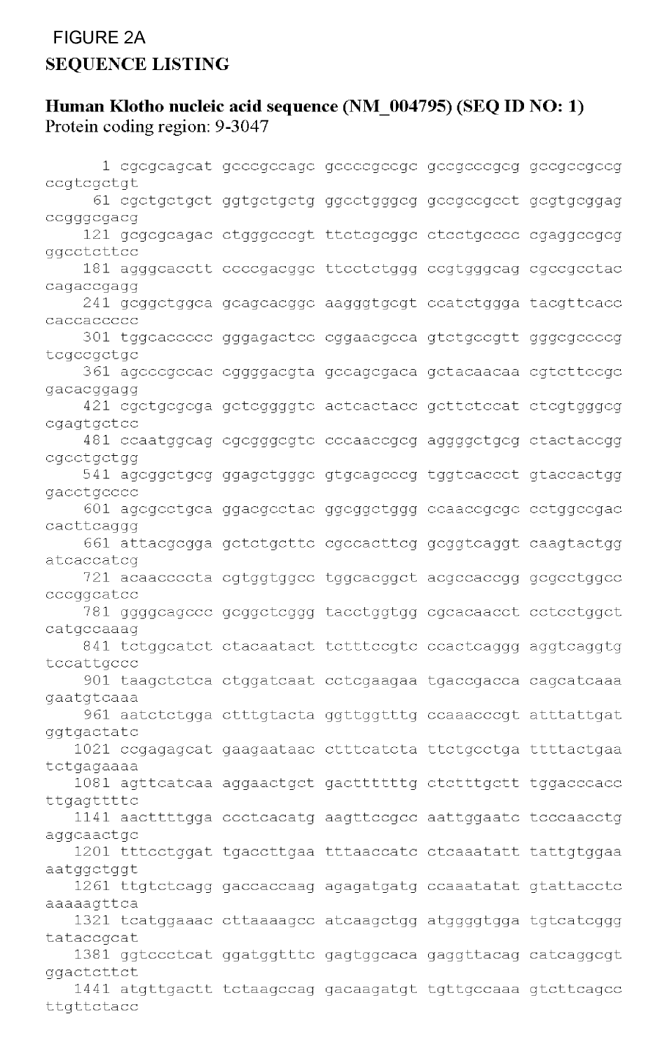

### 4. DETAILED DESCRIPTION

The present invention is directed to methods, kits and compositions for preventing or treating age-related conditions and metabolic disorders. The fusion polypeptides of the invention include a Klotho protein or active fragment thereof. In some embodiments, the fusion polypeptides of the invention include a Klotho protein or an active fragment thereof operatively linked to a fibroblast growth factor polypeptide or active fragment thereof. The Klotho fusion proteins or sKlotho of the present invention are useful in the treatment and prevention of a variety of age-related conditions including sarcopenia, skin atrophy, muscle wasting, brain atrophy, atherosclerosis, arteriosclerosis, pulmonary emphysema, osteoporosis, osteoarthritis, immunologic incompetence, high blood pressure, dementia, Huntington's disease, Alzheimer's disease, cataracts, age-related macular degeneration, prostate cancer, stroke, diminished life expectancy, memory loss, wrinkles, impaired kidney function, and age-related hearing loss; and metabolic disorders including Type II Diabetes, Metabolic Syndrome, hyperglycemia, and obesity.

The present invention, is based at least in part, on the finding that despite the physical constraints (e.g., large size of both the Klotho and FGF polypeptides) the Klotho-FGF fusion polypeptides are highly effective in activating an FGF receptor. This finding is unexpected given that fusion of these two proteins would likely interfere with the heterodimerization and thus the activities of the proteins; e.g., the binding domains of the proteins may be perturbed by the fusion or the proteins may be misoriented spatially if put together in a “cis” formation.

The Klotho-FGF fusion polypeptides described herein are advantageous because they allow the administration of a single therapeutic protein that has enhanced activity compared to Klotho or FGF administered alone or together as separate polypeptides. The use of Klotho and FGF as a single fusion polypeptide rather than as two separate polypeptides (i.e., a Klotho polypeptide and a separate FGF polypeptide) is more effective at activating the FGF receptor.

### Definitions

“Klotho polypeptide”, “Klotho protein”, or “Klotho” as used herein, includes active fragments, derivatives, mimetics, variants and chemically modified compounds or hybrids thereof of wild-type “Klotho”. A Klotho active fragment has the ability to bind to an FGF polypeptide. Generally, a Klotho active polypeptide contains at least a Klotho subdomain (e.g., KL-D1 and KL-D2). Wild-type Klotho has the amino acid sequence as is found in nature. Exemplary Klotho polypeptides suitable for use with the present invention include alpha-Klotho (SEQ ID NO: 2) and beta-Klotho (SEQ ID NO: 4). Nucleotide and amino acid sequences of the alpha-Klotho and beta-Klotho are found in the GenBank database at Accession No. NM_004795; NP_004786 and NM_175737; NP_783864, respectively. Klotho polypeptides include those described in U.S. Pat. No. 6,579,850, the content of which is herein incorporated by reference in its entirety. The Klotho polypeptides include those from other species besides humans, including alpha-Klotho from mouse (NP_038851), rat (NP_112626), rabbit (NP_001075692) and beta-Klotho from mouse (NP_112457). Species predicted to have alpha-Klotho include chimpanzee (XP_522655), macaque (XP_001101127), horse (XP_001495662), cow (XP_001252500), platypus (XP_001510981), and chicken (XP_417105). Species predicted to have beta-Klotho include chimpanzee (XP_526550), macaque (XP_001091413), horse (XP_001495248), dog (XP_536257), rat (XP_001078178), platypus (XP_001512722), and chicken (XP_423224). The Klotho polypeptides have an amino acid sequence that is substantially identical to the amino acid sequence of SEQ ID NO: 2 or SEQ ID NO:4; i.e., at least 70%, 75%, 80%, 85%, 90%, 95%, 96%, 97%, 98%, 99% or more identical at the amino acid sequences of SEQ ID NO:2 or SEQ ID NO:4, or active fragment thereof.

“Fusion polypeptide” or “fusion protein”, as used herein, shall mean a polypeptide comprising two or more different polypeptides or active fragments thereof that are not naturally present in the same polypeptide. In some embodiments, the two or more different polypeptides are operatively linked together covalently, e.g., chemically linked or fused in frame by a peptide bond. As used herein a “Klotho fusion polypeptide” is a fusion polypeptide which includes an amino acid sequence from a Klotho polypeptide or active fragment thereof

“Fibroblast growth factor” and “FGF” are used interchangeably herein and shall refer to polypeptides that regulate cell proliferation, migration, differentiation, homeostasis, tissue repair and response to injury in an animal, including a human subject. FGFs have the ability to bind to a fibroblast growth factor receptor and regulate its activity, including autophosphorylation of FGFR, phosphorylation of FRS2 (FGF receptor substrate 2) and ERK1/2 (extracellular signal-regulated protein kinase 112), and activating Egr-1 (early growth response-1). The term “FGF” includes active fragments, derivatives, mimetics, variants and chemically modified compounds or hybrids thereof of wild-type “FGF”, e.g., as known in the art and as described in U.S. Pat. No. 7,223,563 and U.S. Pat. No. 7,259,248, the contents of which are incorporated by reference in their entirety. Wild-type FGF has an amino acid sequence as is found in nature. Exemplary fibroblast growth factors suitable for use with the present invention include fibroblast growth factor-19 (FGF19; SEQ ID NO: 31), fibroblast growth factor-21 (FGF21; SEQ ID NO: 33), and fibroblast growth factor-23 (FGF23; SEQ ID NO: 35). The FGF polypeptides include those from other species besides humans, including murine FGFs. Generally, FGF polypeptides have an amino acid sequence that is substantially identical to the amino acid sequence of SEQ ID NO: 31, SEQ ID NO:33 or SEQ ID NO:35; i.e., having an amino acid sequence is which is at least 70%, 75%, 80%, 85%, 90%, 95%, 96%, 97%, 98%, 99% or more identical to the amino acid sequences of SEQ ID NO: 31 SEQ ID NO:33 or SEQ ID NO:35, or active fragments thereof

The term “FGF”, includes active fragments of the full-length polypeptide. Active FGF fragments that are able to bind to their corresponding FGF receptors are known in the art and also contemplated for use in the present invention. One skilled in the art would appreciate, based on the sequences disclosed herein, that overlapping fragments of the FGFs can be generated using standard recombinant technology, for example, that described in Sambrook et al. (1989, Molecular Cloning: A Laboratory Manual, Cold Spring Harbor Laboratory Press, New York) and Ausubel et al. (1997, Current Protocols in Molecular Biology, Green & Wiley, New York). One skilled in the art would appreciate, based on the disclosure presented herein, that the biological activity of FGF fragments could be tested by methods well known in the art and described herein, including binding to the FGF receptor. Similarly, cell culture models which possess the necessary FGF signal transduction machinery (i.e. FGF receptor) may be transfected with FGF fragments and subsequently tested for alterations in FGF signaling, relative to wild type FGF.

FGFs are grouped into seven subfamilies based on the homology of the FGF core homology domain (approximately 120 amino acids long), which is flanked by N- and C-terminal sequences that are highly variable in both length and primary sequence, particularly among different FGF subfamilies (Goetz et al., Molecular and Cellular Biology, 2007, Vol 27, 3417-3428). An FGF active polypeptide generally contains at least an FGF core homology domain. In some embodiments, an FGF active polypeptide may contain, in addition to an FGF core homology domain, flanking sequences which may confer additional specificity in binding FGF receptors. FGF19, FGF21, and FGF23 are grouped in the FGF19 subfamily because the core region of these ligands share high sequence identity relative to other FGFs (FGF19 v. FGF21: 38% identity; FGF19 v. FGF23: 36% identity). FGF19 subfamily members act analogously to signaling molecules of the endocrine system and regulate diverse physiological processes uncommon to classical FGFs (e.g., FGF19: energy and bile acid homeostasis; FGF21: glucose and lipid metabolism; and FGF 23: phosphate and vitamin D homeostasis).

“Fibroblast growth factor receptor” and “FGFR” as used herein refer to any one of FGFRs 1-4 known in the art, or splice variants thereof(e.g., FGFR1c). Exemplary fibroblast growth factor receptors suitable for use with the present invention include fibroblast growth factor receptor-19 (e.g., FGFR4-beta Klotho), fibroblast growth factor receptor-21 (e.g., FGFR1c-alpha Klotho), and fibroblast growth factor receptor-23 (e.g., FGFR1c-alpha Klotho, FGFR3-alpha Klotho, FGFR4-alpha Klotho).

“Extracellular domain”, as used herein, refers to the fragment of a transmembrane protein existing outside of a cell (e.g., not including the intracellular or transmembrane region). The “extracellular domain of the Klotho protein”, “soluble Klotho”, or “sKlotho” (e.g., SEQ ID NO: 7; SEQ ID NO: 39), refers to an extracellular domain of the Klotho polypeptide that is capable of binding a fibroblast growth factor, and/or capable of enabling the binding of a fibroblast growth factor to a fibroblast growth factor receptor by binding to the fibroblast growth factor. The Klotho extracellular domain corresponds to amino acid residues 28-982 of the full length alpha Klotho sequence (SEQ ID NO: 2) and to amino acid residues 52-997 of the full length beta Klotho sequence (SEQ ID NO:4).

“Extracellular subdomain of Klotho protein” and “extracellular subdomain of Klotho protein” are used interchangeably herein and shall refer to a region in the extracellular domain of the Klotho polypeptide that is capable of binding a fibroblast growth factor, and/or is capable of enabling the binding of a fibroblast growth factor to a fibroblast growth factor receptor by binding to the fibroblast growth factor. The Klotho extracellular domain has two homologous subdomains that are repeated, i.e., KL-D1 (SEQ ID NO: 5) and KL-D2 (SEQ ID NO: 6). KL-D1 and KL-D2 correspond respectively to amino acid residues 58-506 and 517-953 of the full length alpha Klotho polypeptide (SEQ ID NO: 2) and respectively to amino acid residues 77-508 and 571-967 of the full length beta Klotho polypeptide (SEQ ID NO:4) and are suitable for use with the present invention. Generally, a polypeptide that contains at least one Klotho subdomain is a Klotho active polypeptide. The Klotho extracellular subdomain for use with the polypeptide of the invention may be an alpha Klotho or beta Klotho KL-D1 domain with an amino acid sequence that is substantially identical to the amino acid sequence of SEQ ID NO: 5 or SEQ ID NO: 37, respectively. Further, the Klotho KL-D1 domain may have an amino acid sequence that is at least 70%, 75%, 80%, 85%, 90%, 95%, 96%, 97%, 98%, 99% or more identical to the amino acid sequence of SEQ ID NO: 5 or SEQ ID NO: 37. The Klotho extracellular subdomain may also be an alpha or beta Klotho polypeptide KL-D2 domain that is substantially identical to the amino acid sequence of SEQ ID NO: 6 or SEQ ID NO: 38, respectively. In a further embodiment, the KL-D2 domain has an amino acid sequence that is at least at least 70%, 75%, 80%, 85%, 90%, 95%, 96%, 97%, 98%, 99% or more identical to the amino acid sequence of SEQ ID NO: 6 or SEQ ID NO: 38.

“Signal peptide”, as used herein, shall mean a peptide chain (3-60 amino acids long) that directs the post-translational transport of a protein to the endoplasmic reticulum and may be cleaved off Exemplary signal peptides suitable for use with the present invention include the Klotho signal peptide (SEQ ID NO:19) and the IgG signal peptide (SEQ ID NO:20).

“Linker”, as used herein, shall mean a functional group (e.g., chemical or polypeptide) that covalently attaches two or more polypeptides or nucleic acids so that they are connected with one another. As used herein, a “peptide linker” refers to one or more amino acids used to couple two proteins together (e.g., to couple the extracellular domain of Klotho and fibroblast growth factor-23). Peptide linkers suitable for use with the present invention include, but are not limited to, polypeptides with amino acid sequences represented by SEQ ID NO:8, SEQ ID NO:9, SEQ ID NO: 10, SEQ ID NO:11, SEQ ID NO:12, SEQ ID NO:13, SEQ ID NO:14, SEQ ID NO: 15, SEQ ID NO: 16, SEQ ID NO: 17 and SEQ ID NO: 18.

“Operatively linked”, as used herein, shall mean the linking of two or more biomolecules so that the biological functions, activities, and/or structure associated with the biomolecules are at least retained. In reference to polypeptides, the term means that the linking of two or more polypeptides results in a fusion polypeptide that retains at least some of the respective individual activities of each polypeptide component. The two or more polypeptides may be linked directly or via a linker. In reference to nucleic acids, the term means that a first polynucleotide is positioned adjacent to a second polynucleotide that directs transcription of the first polynucleotide when appropriate molecules (e.g., transcriptional activator proteins) are bound to the second polynucleotide.

“Specifically binds”, as used herein, shall refer to the ability of a first molecule to bind to a target molecule out of many, different types of molecules to which it may be exposed because of the ability of the first molecule to adopt a particular structure conducive to forming noncovalent interactions between itself and the other target molecule. The first molecule binds to the target forming a stable complex while there is substantially less recognition, contact, or complex formation of the first molecule with any other non-specific molecules.

“Polypeptide variant” or “protein variant”, as used herein, refers to polypeptides in which one or more amino acids have been substituted by different amino acids from a reference sequence. It is well understood in the art that some amino acids may be substituted by others with broadly similar properties without changing the nature of the activity of the polypeptide (conservative substitutions) as described hereinafter. These terms also encompass polypeptides in which one or more amino acids have been added or deleted, or replaced with different amino acids, e.g., protein isoforms. An exemplary variant of fibroblast growth factor-23 suitable for use with the present invention is the fibroblast growth factor-23 variant (R179Q).

“Pharmaceutical composition”, as used herein, shall mean a composition containing a compound (e.g., a fusion polypeptide of the invention) that may be administered to treat or prevent a disease or disorder in an individual.

“Individual” or “subject”, as used herein, shall refer to a mammal, including, but not limited to, a human or non-human mammal, such as a bovine, equine, canine, ovine, or feline.

“Treat”, as used herein, shall mean decrease, suppress, attenuate, diminish, arrest, or stabilize the development or progression of a disease. In the context of the invention, the administration of the polypeptides of the invention may be used to treat age-related conditions, including sarcopenia, skin atrophy, muscle wasting, brain atrophy, atherosclerosis, arteriosclerosis, pulmonary emphysema, osteoporosis, osteoarthritis, immunologic incompetence, high blood pressure, dementia, Huntington's disease, Alzheimer's disease, cataracts, age-related macular degeneration, prostate cancer, stroke, diminished life expectancy, memory loss, wrinkles, impaired kidney function, and age-related hearing loss; and metabolic disorders, including Type II Diabetes, Metabolic Syndrome, hyperglycemia, and obesity.

“Prevent”, as used herein, shall refer to a decrease in the occurrence of a disorder or decrease in the risk of acquiring a disorder or its associated symptoms in a subject. In the context of the invention, the administration of the polypeptides of the invention may be used to prevent age-related conditions, including sarcopenia, skin atrophy, muscle wasting, brain atrophy, atherosclerosis, arteriosclerosis, pulmonary emphysema, osteoporosis, osteoarthritis, immunologic incompetence, high blood pressure, dementia, Huntington's disease, Alzheimer's disease, cataracts, age-related macular degeneration, prostate cancer, stroke, diminished life expectancy, memory loss, wrinkles, impaired kidney function, and age-related hearing loss; and metabolic disorders, including Type II Diabetes, Metabolic Syndrome, hyperglycemia, and obesity. The prevention may be complete, e.g., the total absence of an age-related condition or metabolic disorder. The prevention may also be partial, such that the likelihood of the occurrence of the age-related condition or metabolic disorder in a subject is less likely to occur than had the subject not received the present invention.

“Disease”, as used herein, shall mean any condition or disorder that damages or interferes with the normal function of a cell, tissue, or organ.

“Age-related condition”, as used herein, shall mean any disease or disorder whose incidence in a population or severity in an individual correlates with the progression of age. In one embodiment, the age-related condition is a disease or disorder whose incidence is at least 1.5 fold higher among human individuals greater than 60 years of age relative to human individuals between the ages of 30-40 and in a selected population of greater than 100,000 individuals. Age-related conditions relevant to the present invention include, but are not limited to, sarcopenia, skin atrophy, muscle wasting, brain atrophy, atherosclerosis, arteriosclerosis, pulmonary emphysema, osteoporosis, osteoarthritis, immunologic incompetence, high blood pressure, dementia, Huntington's disease, Alzheimer's disease, cataracts, age-related macular degeneration, prostate cancer, stroke, diminished life expectancy, memory loss, wrinkles, impaired kidney function, and age-related hearing loss.

“Metabolic disorder”, as used herein, shall mean any disease or disorder that damages or interferes with normal function in a cell, tissue, or organ by affecting the production of energy in cells or the accumulation of toxins in a cell, tissue, organ, or individual. Metabolic disorders relevant to the present invention include, but are not limited to, Type II Diabetes, Metabolic Syndrome, hyperglycemia, and obesity.

An “effective dose” or “effective amount” is an amount sufficient to effect a beneficial or desired clinical result. In the context of the invention, it is an amount of a Klotho fusion polypeptide or sKlotho effective to produce the intended pharmacological, therapeutic or preventive result. A therapeutically effective dose results in the prevention or amelioration of the disorder or one or more symptoms of the disorder, (e.g., an age-related condition or metabolic disorder). Therapeutically effective doses will vary depending upon the subject and disease condition being treated, the weight and age of the subject, the severity of the disease condition, the manner of administration and the like which can be readily be determined by one of ordinary skill in the art.

“Klotho nucleic acid molecule”, as used herein is a gene encoding a Klotho protein. An exemplary human Klotho gene is provided at GenBank Accession No. NM_004795 (SEQ ID NO: 1).

“Fragment”, as used herein, refers to a portion of a polypeptide or nucleic acid molecule. This portion contains, preferably, at least 10%, 20%, 30%, 40%, 50%, 60%, 70%, 80%, 90% or more of the entire length of the reference nucleic acid molecule or polypeptide. A fragment may contain 10, 20, 30, 40, 50, 60, 70, 80, 90, or 100, 200, 300, 400, 500, 600, 700, 800, 900, 1000 or up to 3000 nucleotides or amino acids.

The term “substantially identical” refers to a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, 70%, 75%, 80% or 85%, 90%, 91%, 92%, 93%, 94%, 95%, 96%, 97%, 98%, 99% or more identical at the amino acid level or nucleic acid to the sequence used for comparison.

The present invention is directed to methods, kits and compositions for preventing or treating age-related conditions and metabolic disorders. The invention provides a fusion polypeptide having at least one extracellular subdomain of a Klotho protein. In some embodiments, the fusion polypeptides further comprises a fibroblast growth factor or an active fragment thereof The Klotho extracellular domain may be derived from either the alpha or beta Klotho isoforms. Further, although the FGF component of the Klotho fusion polypeptide is described primarily with reference to fibroblast growth factor-19, fibroblast growth factor-21 and fibroblast growth factor-23, it is contemplated that any of the twenty-three known FGFs or an active fragment thereof can be used in practicing the invention.

The extracellular domain of the Klotho protein can include one or both of the KL-D1 and KL-D2 domains of a Klotho protein. In some embodiments, the Klotho fusion polypeptide has at least two extracellular subdomains of a Klotho protein. For example, the two extracellular subdomains can be two KL-D1 domains in tandem repeats, two KL-D2 domains in tandem repeats, or one KL-D1 domain and one KL-D2 domain.

The extracellular subdomain of a Klotho protein and the fibroblast growth factor (or an active fragment thereof) can be operatively linked to one another in a variety of orientations and manners. For example, the extracellular subdomain of the Klotho protein can be operatively linked to the N-terminus of the fibroblast growth factor or alternatively the fibroblast growth factor can be operatively linked to the N-terminus of the at least one extracellular subdomain of the Klotho protein.

The fusion polypeptide of the invention may include one or both of the Klotho extracellular domains, i.e., KL-D1 (SEQ ID NO: 5) and KL-D2 (SEQ ID NO: 6). KL-D1 and KL-D2 correspond respectively to amino acid residues 58-506 and 517-953 of the full length alpha Klotho polypeptide (SEQ ID NO: 2) and to amino acid residues 77-508 and 571-967 of the full length beta Klotho polypeptide (SEQ ID NO:4) and are suitable for use with the present invention. The Klotho fusion polypeptide may have a KL-D1 domain of an alpha Klotho polypeptide having an amino acid sequence that is substantially identical to the amino acid sequence of SEQ ID NO: 5 or of a beta Klotho polypeptide having an amino acid sequence that is substantially identical to the amino acid sequence of SEQ ID NO: 37. Specifically, the Klotho fusion polypeptide may have an amino acid sequence that is at least at least 70%, 75%, 80%, 85%, 90%, 95%, 96%, 97%, 98%, 99% or more identical to SEQ ID NO: 5 or SEQ ID NO: 37. The Klotho fusion polypeptide may have a KL-D2 domain of an alpha Klotho polypeptide with an amino acid sequence that is substantially identical to the amino acid sequence of SEQ ID NO: 6 or of a beta Klotho polypeptide having an amino acid sequence that is substantially identical to the amino acid sequence of SEQ ID NO: 38. Specifically, the Klotho fusion polypeptide may have an amino acid sequence that is at least at least 70%, 75%, 80%, 85%, 90%, 95%, 96%, 97%, 98%, 99% or more identical to SEQ ID NO: 6 or SEQ ID NO: 38, respectively.

In some embodiments, the Klotho fusion polypeptide of the invention is soluble and is capable of binding to an FGF receptor.

The Klotho fusion polypeptides of the invention can contain a polypeptide linker which connects the polypeptide having at least one extracellular subdomain of a Klotho protein and the fibroblast growth factor. Suitable linkers are well known in the art and generally contain several Gly and several Ser residues, e.g., (Gly₄ Ser)₃ (SEQ ID NO: 11), Gly₄ Ser polypeptide (SEQ ID NO: 12), Gly (SEQ ID NO: 13), Gly (SEQ ID NO: 14), Gly Ser (SEQ ID NO: 15), Gly₂ Ser (SEQ ID NO: 16), Ala (SEQ ID NO: 17), and Ala (SEQ ID NO: 18). In some embodiments, the linker will have at least 2 and up to about 30 repeats of an amino acid sequence represented by any one of SEQ ID NO:12, SEQ ID NO:13, SEQ ID NO:14, SEQ ID NO:15, SEQ ID NO:16, SEQ ID NO:17, or SEQ ID NO:18.

When a polypeptide linker is present in the Klotho fusion polypeptide of the invention, the polypeptide having at least one extracellular subdomain of a Klotho protein may be connected by a peptide bond to the N-terminus of the linker polypeptide with the FGF connected by a peptide bond to the C-terminus of the polypeptide linker. Alternatively, the FGF may be connected by a peptide bond to the N-terminus of the linker polypeptide with the polypeptide having at least one extracellular subdomain of Klotho connected by a peptide bond to the C-terminus of the polypeptide linker. A chemical linker can also be used to link the two polypeptides.

The Klotho fusion polypeptide of the invention may include a signal peptide. Exemplary signal peptides for use with the Klotho fusion polypeptide include, but are not limited to the Klotho signal peptide (SEQ ID NO: 8) and the IgG signal peptide (SEQ ID NO: 9).

### 4.1. Klotho and Fibroblast Growth Factor Polypeptides

The Klotho fusion polypeptides of the invention are expected to exhibit biological activities comparable to FGF in nature, such as binding to an FGF receptor and inducing the phosphorylation of an FGF receptor, FRS2 (FGF receptor substrate 2) and ERK1/2 (extracellular signal-regulated protein kinase 112) and activating Egr-1 (early growth response-1) gene. FGF is a secreted peptide growth factor that binds the FGF receptor. The amino acid and nucleic acid sequences of FGF are readily available to those of skill in the art. For example, exemplary nucleotide sequences for FGF19, FGF21, and FGF23 can be found in the GenBank database at Accession numbers: NM_005117, NM_019113, and NM_020638, respectively, and herein as SEQ ID NOs: 30, 32, and 34, respectively. Exemplary amino sequences for FGF19, FGF21, and FGF23 can be found in the GenBank database at Accession numbers: NP_005108, NP_061986, and NP_065689, respectively, and herein as SEQ ID NOs: 31, 35, and 35, respectively. Additionally, FGF may include one or more alterations which aid in the expression of the protein, e.g., the FGF23 (R179Q) variant (SEQ ID NO: 36).

The Klotho protein is a 130 kDa single pass type I transmembrane protein with an extracellular domain and a short cytoplasmic domain. The amino acid and nucleic acid sequences of Klotho are readily available to those of skill in the art. For example, exemplary nucleotide sequences for alpha-Klotho and beta-Klotho can be found in the GenBank database at Accession numbers: NM_004795 and NM_175737, respectively, and herein as SEQ ID NOs: 7 and 8, respectively. Exemplary amino acid sequences for alpha-Klotho and beta-Klotho can be found in the GenBank database at Accession numbers: NP_004786 and NP_783864, respectively, and herein as SEQ ID NOs: 2 and 4, respectively.

The Klotho fusion polypeptide of the invention can bind to a fibroblast growth factor receptor and has an alpha-Klotho or beta-Klotho extracellular domain operatively linked to either fibroblast growth factor-19 (SEQ ID NO: 31), fibroblast growth factor-21 (SEQ ID NO: 33), fibroblast growth factor-23 (SEQ ID NO: 35), or variants thereof (which include fibroblast growth factor-23 variant (R179Q) (SEQ ID NO: 36)).

Specifically, the Klotho fusion polypeptide of the invention may include an alpha-Klotho (SEQ ID NO: 2) which is operatively coupled to fibroblast growth factor-23 (SEQ ID NO: 35) or fibroblast growth factor-23 variant (R179Q) (SEQ ID NO: 36). Additionally, the Klotho fusion polypeptide of the invention may have beta-Klotho (SEQ ID NO: 4), which is operatively coupled to fibroblast growth factor-19 (SEQ ID NO: 31). The Klotho fusion polypeptide of the invention may include a beta-Klotho (SEQ ID NO: 4), which is operatively coupled to fibroblast growth factor-21 (SEQ ID NO: 33).

The invention includes homologs of the various Klotho and FGF genes and proteins encoded by those genes. A “homolog,” in reference to a gene refers to a nucleotide sequence that is substantially identical over at least part of the gene or to its complementary strand or a part thereof, provided that the nucleotide sequence encodes a protein that has substantially the same activity/function as the protein encoded by the gene which it is a homolog of Homologs of the genes described herein can be identified by percent identity between amino acid or nucleotide sequences for putative homologs and the sequences for the genes or proteins encoded by them (e.g., nucleotide sequences for genes encoding Klotho and FGF or their complementary strands). Percent identity may be determined, for example, by visual inspection or by using various computer programs known in the art or as described herein. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-³ and e″¹⁰⁰ indicating a closely related sequence.

As used herein, the terms “homology” and “homologous” are not limited to designate proteins having a theoretical common genetic ancestor, but includes proteins which may be genetically unrelated that have, nonetheless, evolved to perform similar functions and/or have similar structures. Functional homology to the various proteins described herein also encompasses proteins that have an activity of the corresponding protein of which it is a homolog. For proteins to have functional homology, it is not required that they have significant identity in their amino acid sequences, but, rather, proteins having functional homology are so defined by having similar or identical activities. For example, with respect to a Klotho molecule, the polypeptide should have the functional characteristics of binding to an FGF polypeptide and enable the binding of the FGF to an FGFR. With respect to an FGF molecule, the polypeptide should have the functional characteristics of binding to an FGFR and causing the activation of FGFR (e.g., phosphorylation). Assays for assessing FGF binding to the FGF receptor and/or activation of the FGF signaling pathway are known in the art and described herein (See Example 2). Assays for assessing Klotho activity are also known in the art and described herein (e.g., binding to a FGF polypeptide). Proteins with structural homology are defined as having analogous tertiary (or quaternary) structure and do not necessarily require amino acid identity or nucleic acid identity for the genes encoding them. In certain circumstances, structural homologs may include proteins which maintain structural homology only at the active site or binding site of the protein.

In addition to structural and functional homology, the present invention further encompasses proteins having amino acid identity to the various Klotho and FGF amino acid sequences described herein. To determine the percent identity/homology of two amino acid sequences, the sequences are aligned for optimal comparison purposes (e.g., gaps can be introduced in the amino acid sequence of one protein for optimal alignment with the amino acid sequence of another protein). The amino acid residues at corresponding amino acid positions are then compared. When a position in one sequence is occupied by the same amino acid residue as the corresponding position in the other, then the molecules are identical at that position. The percent identity between the two sequences is a function of the number of identical positions shared by the sequences (i.e., % identity=# of identical positions/total# of positions multiplied by 100).

The amino acid sequences of molecules of the invention described herein have an amino acid sequence which is at least about 60%, 70%, 80%, 90%, 95%, 96%, 97%, 98%, 99% or more identical or homologous to an amino acid sequence described herein.

The nucleic acid sequences of molecules of the invention described herein have a nucleotide sequence which hybridizes to or is at least about 60%, 70%, 80%, 90%, 95%, 96%, 97%, 98%, 99% or more identical or homologous to a nucleotide sequence described herein.

Nucleic acid molecules appropriate for use in the fusion polypeptides of the invention may have a Klotho or FGF nucleotide sequence which hybridizes under stringent conditions to the complement of a nucleic acid molecule encoding Klotho or FGF, respectively. As used herein, the term “hybridizes under stringent conditions” is intended to describe conditions for hybridization and washing under which nucleotide sequences at least about 70%, 80%, 85%, 90% or more homologous to each other typically remain hybridized to each other. Such stringent conditions are known to those skilled in the art and can be found in Ausubel et al. Current Protocols in Molecular Biology, Wiley Interscience, New York (2001), 6.3.1-6.3.6. A specific, non-limiting example of stringent hybridization conditions are hybridization in 6× sodium chloride/sodium citrate (SSC) at about 45° C., followed by one or more washes in 0.2×SSC, 0.1% SDS at 50-65° C.

### 4.2. Klotho-FGF Fusion Polypeptides of the Invention

In some embodiments of the invention, a Klotho fusion polypeptide has a polypeptide chain having a first polypeptide sequence of a Klotho polypeptide or an active fragment thereof and a second polypeptide sequence encoding FGF or an active fragment thereof.

The invention includes fusion polypeptides which are at least about 95% or more homologous to an amino acid sequence presented in SEQ ID NO:19-28. The amino acid sequence of SEQ ID NO: 19 encodes a Klotho fusion polypeptide having a Klotho extracellular domain N-terminally linked to the FGF23 (R179Q) variant (SEQ ID NO: 36). The amino acid sequence of SEQ ID NO: 20 encodes a Klotho fusion polypeptide having an IgG signal peptide N-terminally linked to a Klotho extracellular domain lacking a signal peptide N-terminally linked to the FGF23 (R179Q) variant. The amino acid sequence of SEQ ID NO: 21 encodes a Klotho fusion polypeptide having a KL-D1 extracellular subdomain N-terminally linked to the FGF23 (R179Q) variant. The amino acid sequence of SEQ ID NO: 22 encodes a Klotho fusion polypeptide having a KL-D2 extracellular subdomain N-terminally linked to the FGF23 (R179Q) variant. The amino acid sequence of SEQ ID NO: 23 encodes a Klotho fusion polypeptide having two KL-D1 extracellular subdomains N-terminally linked to the FGF23 (R179Q) variant. The amino acid sequence of SEQ ID NO: 24 encodes a Klotho fusion polypeptide having two KL-D2 extracellular subdomains N-terminally linked to the FGF23 (R179Q) variant. The amino acid sequence of SEQ ID NO: 25 encodes a Klotho fusion polypeptide having the FGF23 (R179Q) variant N-terminally linked to a Klotho extracellular domain. The amino acid sequence of SEQ ID NO: 26 encodes a Klotho fusion polypeptide having the FGF23 (R179Q) variant N-terminally linked to a KL-D1 extracellular subdomain. The amino acid sequence of SEQ ID NO: 27 encodes a Klotho fusion polypeptide having the FGF23 (R179Q) variant N-terminally linked to a KL-D2 extracellular subdomain. The amino acid sequence of SEQ ID NO: 28 encodes a Klotho fusion polypeptide having the FGF23 (R179Q) variant N-terminally linked to two KL-D1 extracellular subdomains. The amino acid sequence of SEQ ID NO: 29 encodes a Klotho fusion polypeptide having the FGF23 (R179Q) variant N-terminally linked to two KL-D2 extracellular subdomains.

The Klotho fusion polypeptide of the invention may include an amino acid sequence which is at least about 95% identical to the amino acid sequence set forth in SEQ ID NO:7. The amino acid sequence of SEQ ID NO: 7 encodes a Klotho extracellular domain lacking a signal peptide.

The subject fusion proteins are described herein and can be made using methods known in the art. For example, the fusion polypeptides of the invention may be constructed as described in U.S. Pat. No. 6,194,177. The use of Klotho polypeptides is described in U.S. Pat. No. 6,579,850. The use of FGF nucleic acid molecules is described in U.S. Pat. No. 7,223,563.

In some embodiments, a nucleic acid molecule encoding the Klotho is cloned by PCR and ligated, in frame, with a nucleic acid molecule encoding FGF. The nucleic acid encoding the Klotho-FGF fusion polypeptide is operatively linked to a promoter to allow for expression. The nucleic acid molecule encoding the fusion polypeptide is subsequently transfected into a host cell for expression. The sequence of the final construct can be confirmed by sequencing.

When preparing the fusion proteins of the present invention, a nucleic acid molecule encoding an extracellular subdomain of Klotho will be fused in frame to the nucleic acid molecule encoding FGF. Expression of the resulting nucleic acid molecule results in the extracellular subdomain of Klotho being fused N-terminal in relation to the FGF polypeptide. Fusions are also possible in which the extracellular subdomain of Klotho is fused C-terminal in relation to the FGF polypeptide. Methods for making fusion proteins are well known in the art.

The fusion polypeptides of the invention have at least two polypeptides that are covalently linked, in which one polypeptide comes from one protein sequence or domain, e.g., Klotho, and the other polypeptide comes from another protein sequence or domain, e.g., FGF. Klotho and FGF, of the fusion polypeptides of the invention, can be joined by methods well known to those of skill in the art. These methods include both chemical and recombinant means.

Nucleic acids encoding the domains to be incorporated into the fusion polypeptides of the invention can be obtained using routine techniques in the field of recombinant genetics. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994-1999). In nucleic acids encoding a Klotho fusion polypeptide of the invention, the nucleic acid sequence encoding alpha-Klotho or beta-Klotho, represented by SEQ ID NO: 1 and SEQ ID NO: 3, respectively, may be used. In nucleic acids encoding a Klotho fusion polypeptide, the nucleic acid sequence encoding FGF19, FGF21, or FGF23, represented by SEQ ID NO: 30, SEQ ID NO: 32 and SEQ ID NO: 34, respectively, may be used. Nucleic acid sequences of molecules of the invention described herein comprise a nucleotide sequence which hybridizes to or is at least about 60%, 70%, 80%, 90%, 95%, 96%, 97%, 98%, 99% or more identical or homologous to SEQ ID NO: 1, SEQ ID NO:3, SEQ ID NO: 30, SEQ ID NO: 32, or SEQ ID NO: 34.

Nucleic acid sequences that encode Klotho and FGF peptides can be obtained using any of a variety of methods. For example, the nucleic acid sequences encoding the polypeptides may be cloned from eDNA and genomic DNA libraries by hybridization with probes, or isolated using amplification techniques with oligonucleotide primers. More commonly, amplification techniques are used to amplifY and isolate the Klotho and FGF sequences using a DNA or RNA template (see, e.g., Dieffenfach & Dveksler, PCR Primers: A Laboratory Manual (1995)). Alternatively, overlapping oligonucleotides can be produced synthetically and joined to produce one or more of the domains. Nucleic acids encoding Klotho or FGF can also be isolated from expression libraries using antibodies as probes.

According to the present invention, Klotho and FGF can be linked either directly or via a covalent linker, including amino acid linkers, such as a polyglycine linker, or another type of chemical linker, including, carbohydrate linkers, lipid linkers, fatty acid linkers, polyether linkers, such as PEG, etc. (See for example, Hermanson, Bioconjugate techniques (1996)). The polypeptides forming the fusion/fusion polypeptide are typically linked C-terminus to N-terminus, although they can also be linked C-terminus to C-terminus, N-terminus to N-terminus, or N-terminus to C-terminus. One or more polypeptide domains may be inserted at an internal location within a fusion polypeptide of the invention. The polypeptides of the fusion protein can be in any order. The fusion polypeptides may be produced by covalently linking a chain of amino acids from one protein sequence, e.g., an extracellular subdomain of Klotho, to a chain of amino acids from another protein sequence, e.g., FGF, by preparing a recombinant polynucleotide contiguously encoding the fusion protein. The different chains of amino acids in a fusion protein may be directly spliced together or may be indirectly spliced together via a chemical linking group or an amino acid linking group. The amino acid linking group can be about 200 amino acids or more in length, or generally 1 to 100 amino acids. In some embodiments, proline residues are incorporated into the linker to prevent the formation of significant secondary structural elements by the linker. Linkers can often be flexible amino acid subsequences that are synthesized as part of a recombinant fusion protein. Such flexible linkers are known to persons of skill in the art.

According to the present invention, the amino acid sequence of an extracellular subdomain of Klotho or a fragment thereof may be linked to the FGF via a peptide linker. Exemplary peptide linkers are well known in the art and described herein. For example, peptide linkers generally include several Gly and several Ser residues, such as: (Gly₄ Ser)₃ (SEQ ID NO: 11), Gly₄ Ser polypeptide (SEQ ID NO: 12), Gly (SEQ ID NO: 13), Gly (SEQ ID NO: 14), Gly Ser (SEQ ID NO: 15), Gly₂ Ser (SEQ ID NO: 16), Ala (SEQ ID NO: 17), and Ala (SEQ ID NO: 18). Specifically, a peptide linker for use in a fusion protein of the invention may act as a flexible hinge.

The signal sequence of Klotho or FGF may be excluded prior to incorporation of Klotho into a fusion protein of the invention. The signal sequence for Klotho or FGF of the fusion protein may be included, e.g., the polypeptide represented by SEQ ID NO: 19. However, such sequences may also be omitted and replaced with the signal sequence of a different protein, e.g., the IgG signal sequence (SEQ ID NO: 9). Generally, the pharmaceutical compositions of the invention will contain the mature form of Klotho and FGF.

Generally, introns are excluded from either one or both the Klotho or the FGF moieties prior to incorporation into a fusion polypeptide.

The fusion polypeptides of the invention may include one or more polymers covalently attached to one or more reactive amino acid side chains. By way of example, not limitation, such polymers include polyethylene glycol (PEG), which can be attached to one or more free cysteine sulfhydryl residues, thereby blocking the formation of disulfide bonds and aggregation when the protein is exposed to oxidizing conditions. In addition, PEGylation of the fusion polypeptides of the invention is expected to provide such improved properties as increased half-life, solubility, and protease resistance. The fusion polypeptides of the invention may alternatively be modified by the covalent addition of polymers to free amino groups such as the lysine epsilon or the N-terminal amino group. Preferred cysteine and lysines for covalent modification will be those not involved in receptor binding, heparin binding, or in proper protein folding. It will be apparent to one skilled in the art that the methods for assaying the biochemical and/or biological activity of the fusion polypeptides may be employed in order to determine if modification of a particular amino acid residue affects the activity of the protein as desired. Other similar suitable modifications are contemplated and known in the art.

The invention is also directed to the expression of a fusion polypeptide that is at least about 95% or more homologous to an amino acid sequence presented in SEQ ID NO:19-28.

### 4.3. Expression of Fusion Polypeptides of the Invention

In order to express the fusion protein of the invention, DNA molecules obtained by any of the methods described herein or those that are known in the art, can be inserted into appropriate expression vectors by techniques well known in the art. For example, a double stranded cDNA can be cloned into a suitable vector by homopolymeric tailing or by restriction enzyme linking involving the use of synthetic DNA linkers or by blunt-ended ligation. DNA ligases are usually used to ligate the DNA molecules and undesirable joining can be avoided by treatment with alkaline phosphatase.

Therefore, the invention includes vectors (e.g., recombinant plasmids and bacteriophages) that include nucleic acid molecules (e.g., genes or recombinant nucleic acid molecules encoding genes) as described herein. The term “recombinant vector” includes a vector (e.g., plasmid, phage, plasmid, virus, cosmid, fosmid, or other purified nucleic acid vector) that has been altered, modified or engineered such that it contains greater, fewer or different nucleic acid sequences than those included in the native or natural nucleic acid molecule from which the recombinant vector was derived. For example, a recombinant vector may include a nucleotide sequence encoding a Klotho-FGF23 fusion operatively linked to regulatory sequences, e.g., promoter sequences, terminator sequences and/or artificial ribosome binding sites (RBSs), as defined herein. Recombinant vectors which allow for expression of the genes or nucleic acids included in them are referred to as “expression vectors.”

For eukaryotic hosts, different transcriptional and translational regulatory sequences may be employed, depending on the nature of the host. They may be derived from viral sources, such as adenovirus, bovine papilloma virus, Simian virus or the like, where the regulatory signals are associated with a particular gene which has a high level of expression. Examples include, but are not limited to, the TK promoter of the Herpes virus, the SV40 early promoter, the yeast ga14 gene promoter, etc. Transcriptional initiation regulatory signals may be selected which allow for repression or activation, so that expression of the genes can be modulated.

In some of the molecules of the invention described herein, one or more DNA molecules having a nucleotide sequence encoding one or more polypeptide chains of a fusion polypeptide are operatively linked to one or more regulatory sequences, which are capable of integrating the desired DNA molecule into a host cell. Cells which have been stably transformed by the introduced DNA can be selected, for example, by introducing one or more markers which allow for selection of host cells which contain the expression vector. A selectable marker gene can either be linked directly to a nucleic acid sequence to be expressed, or be introduced into the same cell by co-transfection. Additional elements may also be needed for optimal synthesis of proteins described herein. It would be apparent to one of ordinary skill in the art which additional elements to use.

Factors of importance in selecting a particular plasmid or viral vector include, but are not limited to, the ease with which recipient cells that contain the vector are recognized and selected from those recipient cells which do not contain the vector; the number of copies of the vector which are desired in a particular host; and whether it is desirable to be able to “shuttle” the vector between host cells of different species.

Once the vector(s) is constructed to include a DNA sequence for expression, it may be introduced into an appropriate host cell by one or more of a variety of suitable methods that are known in the art, including but not limited to, for example, transformation, transfection, conjugation, protoplast fusion, electroporation, calcium phosphate-precipitation, direct microinjection, etc.

Host cells may either be prokaryotic or eukaryotic. Examples of eukaryotic host cells include, for example, mammalian cells, such as human, monkey, mouse, and Chinese hamster ovary (CHO) cells. Such cells facilitate post-translational modifications of proteins, including, for example, correct folding or glycosylation. Additionally, yeast cells can also be used to express fusion polypeptides of the invention. Like most mammalian cells, yeast cells also enable post-translational modifications of proteins, including, for example, glycosylation. A number of recombinant DNA strategies exist which utilize strong promoter sequences and high copy number plasmids that can be utilized for production of proteins in yeast. Yeast transcription and translation machinery can recognize leader sequences on cloned mammalian gene products, thereby enabling the secretion of peptides bearing leader sequences (i.e., pre-peptides). A particularly preferred method of high-yield production of the fusion polypeptides of the invention is through the use of dihydrofolate reductase (DHFR) amplification in DHFR-deficient CHO cells, by the use of successively increasing levels of methotrexate as described in U.S. Pat. No. 4,889,803. The polypeptide obtained may be in a glycosylated form.

After the introduction of one or more vector(s), host cells are usually grown in a selective medium, which selects for the growth of vector-containing cells. Purification of the recombinant proteins can be carried out by any of the methods known in the art or described herein, for example, any conventional procedures involving extraction, precipitation, chromatography and electrophoresis. A further purification procedure that may be used for purifying proteins is affinity chromatography using monoclonal antibodies which bind a target protein. Generally, crude preparations containing a recombinant protein are passed through a column on which a suitable monoclonal antibody is immobilized. The protein usually binds to the column via the specific antibody while the impurities pass through. After washing the column, the protein is eluted from the gel by changing pH or ionic strength, for example.

### 4.4. Assays for Assessing Fusion Polypeptide Activity

Assays described herein (See Example 2) and those known in the art can be used for detecting Klotho or FGF activity of the fusion polypeptides of the invention. Suitable activity assays include receptor binding assays, cellular proliferation assays and cell signaling assays. For example, a binding assay which may be used for determining whether a fusion polypeptide has Klotho or FGF activity includes, assaying the binding of a fusion polypeptide to an FGF receptor. FGF receptor binding assays include, but are not limited to, both competitive and non-competitive assay. For example, FGF receptor binding can be detected by contacting cells expressing an FGF receptor with a labeled FGF (for example, radio-active label) and increasing concentrations of an unlabeled Klotho-FGF fusion polypeptide. The two ligands that compete for binding to the same receptor are added to a reaction mixture containing the cell. The cells are subsequently washed and labeled FGF is measured. A decrease in the amount of the labeled FGF to its receptor in the presence of the unlabeled fusion polypeptide is indicative of binding of the Klotho-FGF fusion polypeptide to the receptor. Alternatively, the Klotho-FGF fusion polypeptide may be labeled and direct binding of the fusion polypeptide to the cell is detected.

Klotho or FGF activity can also be measured by determining whether the fusion polypeptide induces a cellular response. For example, in some embodiments, an assay for detecting the biological activity of a Klotho-FGF fusion polypeptide involves contacting cells which express an FGF receptor with a fusion polypeptide, assaying a cellular response such as, for example, cell proliferation or Egr-1 activation, myotube diameter in C2C12 cells, and comparing the cellular response in the presence and absence of the fusion polypeptide. An increase in the cellular response in the presence of the fusion polypeptide complex relative to the absence indicates that the fusion polypeptide has biological activity. Also, an increase in a downstream signaling event from the receptor can also be measured as indicia of biological activity (e.g., phosphorylation of FGFR, FRS2, ERK1/2, p70S6K etc.).

### 4.5 Pharmaceutical Compositions and Methods of Treatment

The invention also pertains to pharmaceutical compositions containing one or more fusion polypeptides of the invention and a pharmaceutically acceptable diluent or carrier. The pharmaceutical compositions can further include a pharmaceutically effective dose of heparin. Such pharmaceutical compositions may be included in a kit or container. Such kit or container may be packaged with instructions pertaining to the extended in vivo half-life or the in vitro shelf life of the fusion polypeptides. Optionally associated with such kit or container(s) can be a notice in the form prescribed by a governmental agency regulating the manufacture, use or sale of pharmaceuticals or biological products, which notice reflects approval by the agency of manufacture, use or sale for human administration. Such compositions may be used in methods of treating, preventing, or ameliorating a disease or a disease symptom (e.g., age-related condition or metabolic disorder) in a patient, preferably a mammal and most preferably a human, by administering the pharmaceutical composition to the patient.

In general, a therapeutically effective amount of a pharmaceutical composition of the invention is from about 0.0001 mgikg to 0.001 mgikg; 0.001 mgikg to about 10 mgikg body weight or from about 0.02 mglkg to about 5 mglkg body weight. Commonly, a therapeutically effective amount of a fusion polypeptide is from about 0.001 mg to about 0.01 mg, about 0.01 mg to about 100 mg, or from about 100 mg to about 1000 mg, for example. Preferably, a therapeutically effective amount of a fusion polypeptide is from about 0.001 mglkg to 2 mglkg.

The optimal pharmaceutical formulations for a fusion polypeptide can be determined by one or ordinary skilled in the art depending upon the route of administration and desired dosage. (See, for example, Remington's Pharmaceutical Sciences, 18th Ed. (1990), Mack Publishing Co., Easton, Pa., the entire disclosure of which is hereby incorporated by reference).

The fusion polypeptides of the invention may be administered as a pharmaceutical composition that may be in the form of a solid, liquid or gas (aerosol). Typical routes of administration may include, without limitation, oral, topical, parenteral, sublingual, rectal, vaginal, intradermal and intranasal. Parenteral administration includes subcutaneous injections, intravenous, intramuscular, intraperitoneal, intrapleural, intrasternal injection or infusion techniques. Preferably, the compositions are administered parenterally. More preferably, the compositions are administered intravenously. Pharmaceutical compositions of the invention can be formulated so as to allow a polypeptide of the invention to be bioavailable upon administration of the composition to a subject. Compositions can take the form of one or more dosage units, where, for example, a tablet can be a single dosage unit, and a container of a polypeptide of the invention in aerosol form can hold a plurality of dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of subject (e.g., human), the overall health of the subject, the type of age-related condition or metabolic disorder the subject in need of treatment of, the use of the composition as part of a multi-drug regimen, the particular form of the polypeptide of the invention, the manner of administration, and the composition employed.

The pharmaceutically acceptable carrier or vehicle may be particulate, so that the compositions are, for example, in tablet or powder form. The carrier(s) can be liquid, with the compositions being, for example, an oral syrup or injectable liquid. In addition, the carrier(s) can be gaseous, so as to provide an aerosol composition useful in, e.g., inhalatory administration.

The term “carrier” refers to a diluent, adjuvant or excipient, with which a polypeptide of the invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to a subject, the polypeptides of the invention and pharmaceutically acceptable carriers are sterile. Water is a preferred carrier when the polypeptide of the invention is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The composition may be intended for oral administration, and if so, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as ethyl cellulose, carboxymethylcellulose, microcrystalline cellulose, or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin, a flavoring agent such as peppermint, methyl salicylate or orange flavoring, and a coloring agent.

When the pharmaceutical composition is in the form of a capsule, e.g., a gelatin capsule, it can contain, in addition to materials of the above type, a liquid carrier such as polyethylene glycol, cyclodextrin or a fatty oil.

The pharmaceutical composition can be in the form of a liquid, e.g., an elixir, syrup, solution, emulsion or suspension. The liquid can be useful for oral administration or for delivery by injection. When intended for oral administration, a composition can contain one or more of a sweetening agent, preservatives, dye/colorant and flavour enhancer. In a composition for administration by injection, one or more of a surfactant, preservative, wetting agent, dispersing agent, suspending agent, buffer, stabilizer and isotonic agent can also be included.

The liquid compositions of the invention, whether they are solutions, suspensions or other like form, can also include one or more of the following: sterile diluents such as water for injection, saline solution, preferably physiological saline, Ringer's solution, isotonic sodium chloride, fixed oils such as synthetic mono or diglycerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A parenteral composition can be enclosed in an ampoule, a disposable syringe or a multiple-dose vial made of glass, plastic or other material. Physiological saline is a preferred adjuvant. An injectable composition is preferably sterile.

The pharmaceutical compositions contain an effective amount of a compound of the invention (e.g., fusion polypeptide) such that a suitable dosage will be obtained. The pharmaceutical compositions may contain the known effective amount of the compounds as currently prescribed for their respective disorders.

The route of administration of the polypeptide of the invention used in the prophylactic and/or therapeutic regimens which will be effective in the prevention, treatment, and/or management of a age-related condition or metabolic disorder can be based on the currently prescribed routes of administration for other therapeutics known in the art. The polypeptides of the invention can be administered by any convenient route, for example, by infusion or bolus injection, by absorption through epithelial or mucocutaneous linings (e.g., oral mucosa, rectal and intestinal mucosa, etc.). Administration can be systemic or local. Various delivery systems are known, e.g., microparticles, microcapsules, capsules, etc., and may be useful for administering a polypeptide of the invention. More than one polypeptides of the invention may be administered to a subject. Methods of administration may include, but are not limited to, oral administration and parenteral administration; parenteral administration including, but not limited to, intradermal, intramuscular, intraperitoneal, intravenous, subcutaneous, intranasal, epidural, sublingual, intranasal, intracerebral, intraventricular, intrathecal, intravaginal, transdermal, rectally, by inhalation, or topically to the ears, nose, eyes, or skin.

The polypeptides of the invention may be administered parenterally. Specifically, the polypeptides of the invention may be administered intravenously.

Pulmonary administration can also be employed, e.g., by use of an inhaler or nebulizer, and formulation with an aerosolizing agent, or via perfusion in a fluorocarbon or synthetic pulmonary surfactant. The polypeptides of the invention can also be formulated as a suppository, with traditional binders and carriers such as triglycerides.

The polypeptides of the invention can be delivered in a controlled release system. For example, a pump can be used (see Sefton, CRC Crit. Ref Biomed. Eng. 1987, 14, 201; Buchwald et al., Surgery 1980, 88: 507; Saudek et al., N. Engl. J. Med. 1989, 321: 574). Polymeric materials can also be used for controlled release of the polypeptides of the invention (see Medical Applications of Controlled Release, Langer and Wise (eds.), CRC Pres., Boca Raton, Fla., 1974; Controlled Drug Bioavailability, Drug Product Design and Performance, Smolen and Ball (eds.), Wiley, New York, 1984; Ranger and Peppas, J. Macromol. Sci. Rev. Macromol. Chern. 1983, 23, 61; see also Levy et al., Science 1985, 228, 190; During et al., Ann. Neural., 1989, 25, 351; Howard et al., J. Neurosurg., 1989, 71, 105). Specifically, a controlled-release system can be placed in proximity of the target of the polypeptides of the invention, e.g., the brain, thus requiring only a fraction of the systemic dose (see, e.g., Goodson, in Medical Applications of Controlled Release, supra, vol. 2, 1984, pp. 115-138). Other controlled-release systems discussed in the review by Langer (Science 1990, 249, 1527-1533) can be used.

Polymeric materials used to achieve controlled or sustained release of the polypeptides of the invention are disclosed, e.g., in U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. Preferably, the polymer used in a sustained release formulation is inert, free of leachable impurities, stable on storage, sterile, and biodegradable.

In general, a therapeutically effective amount of a pharmaceutical composition of the invention is from about 0.0001 mglkg to 0.001 mglkg; 0.001 mglkg to about 10 mglkg body weight or from about 0.02 mglkg to about 5 mglkg body weight.

In other embodiments, the prophylactic and/or therapeutic regimen involves administering to a patient one or more doses of an effective amount of a polypeptide of the invention, wherein the dose of an effective amount achieves a plasma level of at least 0.01, ugimL to at least 400 ugimL of the polypeptide of the invention.

A prophylactic and/or therapeutic regimen may involve administering to a patient a plurality of doses of an effective amount of a polypeptide of the invention, wherein the plurality of doses maintains a plasma level of at least 0.01, ugimL, to 400 μg/mL of the polypeptide of the invention. The prophylactic and/or therapeutic regimen may be administered for at least 1 day, 1 month, 2 months, 3 months, 4 months, 5 months, 6 months, 7 months, 8 months or 9 months.

The prophylactic and/or therapeutic regimen may involve administration of a polypeptide of the invention in combination with one or more additional therapeutics. The recommended dosages of the one or more therapeutics currently used for the prevention, treatment, and/or management of an age-related condition or metabolic disorder can be obtained from any reference in the art including, but not limited to, Hardman et al., eds., Goodman & Gilman's The Pharmacological Basis Of Basis Of Therapeutics, 10th ed., Mc-Graw-Hill, New York, 2001; Physician's Desk Reference (60th ed., 2006), which is incorporated herein by reference in its entirety.

The invention includes methods of treating disorders wherein agonistic activity of Klotho protein and FGF are desirable. Examples of such methods of the invention include, but are not limited to age-related condition or metabolic disorders.

The invention includes methods for treating or preventing an age-related condition in an individual. An individual in need of treatment is administered a pharmacologically effective dose of a pharmaceutical composition containing a Klotho fusion polypeptide, having at least one extracellular subdomain of a Klotho protein and a fibroblast growth factor so as to treat or prevent the age-related condition. In some embodiments, the Klotho fusion polypeptide is coadministered with a pharmacologically effective dose of heparin. Age-related conditions include sarcopenia, skin atrophy, muscle wasting, brain atrophy, atherosclerosis, arteriosclerosis, pulmonary emphysema, osteoporosis, osteoarthritis, immunologic incompetence, high blood pressure, dementia, Huntington's disease, Alzheimer's disease, cataracts, age-related macular degeneration, prostate cancer, stroke, diminished life expectancy, memory loss, wrinkles, impaired kidney function, and age-related hearing loss. In some embodiments, the Klotho fusion polypeptide contains at least one extracellular domain of an alpha Klotho protein. In a particular embodiment, a Klotho fusion protein containing at least one extracellular domain of alpha Klotho protein and fibroblast growth factor 23 is administered to an individual in need of treatment for muscle wasting.

The invention is also directed to a method for treating or preventing a metabolic disorder in an individual. An individual in need of treatment is administered a pharmacologically effective dose of a pharmaceutical composition containing a Klotho fusion polypeptide, having at least one extracellular subdomain of a Klotho protein and a fibroblast growth factor so as to treat the metabolic disorder. In some embodiments, the Klotho fusion polypeptide is coadministered with a pharmacologically effective dose of heparin. The method may be used in the treatment or prevention of Type II Diabetes, Metabolic Syndrome, hyperglycemia, and obesity. In a particular embodiment, a Klotho fusion protein containing at least one extracellular domain of a beta-Klotho protein and fibroblast growth factor 21 is administered to an individual in need of treatment for a metabolic disorder.

The invention also provides methods for treating or preventing hyperphosphatemia or calcinosis in an individual. An individual in need of treatment is administered a pharmacologically effective dose of a pharmaceutical composition containing a Klotho fusion polypeptide, having at least one extracellular subdomain of a Klotho protein and a fibroblast growth factor so as to treat hyperphosphatemia or calcinosis. In some embodiments, the Klotho fusion polypeptide is coadministered with a pharmacologically effective dose of heparin. In a particular embodiment, a Klotho fusion protein containing at least one extracellular domain of an alpha Klotho protein and fibroblast growth factor 23 is administered to an individual in need of treatment for a hyperphosphatemia or calcinosis.

The invention is also directed to a method for treating or preventing chronic renal disease or chronic renal failure in an individual. An individual in need of treatment is administered a pharmacologically effective dose of a pharmaceutical composition containing a Klotho fusion polypeptide, having at least one extracellular subdomain of a Klotho protein and a fibroblast growth factor so as to treat chronic renal disease or chronic renal failure. In some embodiments, the Klotho fusion polypeptide is coadministered with a pharmacologically effective dose of heparin. In some embodiments, a Klotho fusion protein containing at least one extracellular domain of an alpha Klotho protein is administered to an individual in need of treatment for chronic renal disease or chronic renal failure.

The invention also includes methods for treating or preventing cancer in an individual. An individual in need of treatment is administered a pharmacologically effective dose of a pharmaceutical composition containing a Klotho fusion polypeptide, having at least one extracellular subdomain of a Klotho protein and a fibroblast growth factor so as to treat cancer. The method may be used in the treatment or prevention of breast cancer. In some embodiments, the Klotho fusion polypeptide is coadministered with a pharmacologically effective dose of heparin. In some embodiments, a Klotho fusion protein containing at least one extracellular domain of an alpha Klotho protein is administered to an individual in need of treatment for cancer.

In methods of treating disorders by administering a pharmaceutical composition containing a Klotho fusion polypeptide, the Klotho fusion polypeptide has at least one extracellular subdomain of a Klotho protein and a fibroblast growth factor. In a particular embodiment, the Klotho fusion protein contains at least one extracellular domain of a beta Klotho protein and fibroblast growth factor 21.

The Klotho fusion polypeptide composition can be administered according to any method of administration known to those of skill in the art and described herein. Preferred methods of administration include subcutaneous or intravenous. Other effective modes of administration are described herein.

### 4.6. Methods of Treatment and Assays for Assessing Efficacy

Methods of the invention which provide administering the Klotho fusion polypeptide to an individual can be used to treat a variety of disorders including an age-related disorder or a metabolic disorder. Without being limited by any particular theory, Klotho/FGF fusion polypeptides may be used to treat disorders in which there is dysregulation of Klotho or FGF. Exemplary disorders include metabolic disorders and age-related disorders. For example, both FGF23 or Klotho knock-out mice display a variety of similar phenotypes including, low physical activity, growth retardation, muscle wasting, skin atrophy, atherosclerosis, short life spans, etc. (See Razzaque and Lanske, J. of Endrocrinology, 194:1-10 (2007), which is herein incorporated by reference).

In particular, Klotho/FGF23 fusion polypeptides of the invention are particularly useful in the treatment of aging-related disorders, including muscle wasting. Without being bound to theory, the ability of Klotho and FGF23 to control mineral (e.g., phosphate and calcium) and vitamin D homeostasis may be the means by which these proteins modulate aging and muscle atrophy.

On the other hand, Klotho/FGF19 fusion polypeptides and Klotho/FGF21 fusion polypeptides of the invention may be used for treating a metabolic disorder. For example, beta-Klotho and FGF19 have been shown to control bile acid homeostasis by regulating cholesterol ?-a-hydroxylase (CYP7A1). A non-limiting example of bile homeostasis disorder is cholestosis. The beta-Klotho and FGF21 have been shown to induce lipolysis in adipocytes and, therefore, reduced fat storage and increased glucose uptake. Non-limiting examples of lipolysis/fat storage disorders are obesity and associated metabolic and cardiovascular diseases.

Based at least in part on the finding that FGF23 is able to stimulate excretion of phosphate in the urine and thereby reduce phosphate levels in the serum, Klotho-FGF23 fusion polypeptides of the invention can be used for treating or preventing hyperphosphatemia or calcinosis in an individual. For example, it has been shown that a homozygous missense mutation in Klotho resulting in a deficiency in Klotho in a patient can cause severe tumoral calcinosis and artery calcification (Ichikawa et al., J. Clin. Invest. 117:2684-2691 (2007), which is herein incorporated by reference). An individual is administered a pharmacologically effective dose of a pharmaceutical composition containing the Klotho fusion polypeptide, having at least one extracellular subdomain of a Klotho protein and a fibroblast growth factor so as to treat or preven hyperphosphatemia or calcinosis. In particular, a Klotho fusion polypeptide containing at least one extracellular domain of an alpha Klotho protein and a fibroblast growth factor is useful for treating hyperphosphatemia or calcinosis.

Klotho fusion polypeptides of the invention can also be used for treating or preventing chronic renal disease or chronic renal failure in an individual. For example, it has been shown that Klotho expression is reduced in kidney of patients with chronic renal failure, compared to that in unaffected kidneys (Koh et al., Biochem. Biophys. Res. Comm. 280:1015-1020 (2001), which is herein incorporated by reference). An individual is administered a pharmacologically effective dose of a pharmaceutical composition containing the Klotho fusion polypeptide, having at least one extracellular subdomain of a Klotho protein and a fibroblast growth factor so as to treat or prevent chronic renal disease or chronic renal failure. In particular, a Klotho fusion polypeptide containing at least one extracellular domain of an alpha Klotho protein is useful for treating chronic renal disease or chronic renal failure.

Klotho fusion polypeptides of the invention can also be used for treating or preventing cancer in an individual. For example, it has been shown that Klotho expression is reduced in breast cancer tissue, compared to normal breast cancer tissue (Wolf et al., Oncogene (2008) advance online publication, which is herein incorporated by reference). An individual is administered a pharmacologically effective dose of a pharmaceutical composition containing the Klotho fusion polypeptide, having at least one extracellular subdomain of a Klotho protein and a fibroblast growth factor so as to treat or prevent cancer or breast cancer. In particular, a Klotho fusion protein containing at least one extracellular domain of an alpha Klotho protein is useful for treating cancer or breast cancer.

Methods for evaluating the efficacy and/or determining the effective dose of a Klotho fusion polypeptide of the invention on an age-related disorder or metabolic disorder include organismal based assays, e.g., using a mammal (e.g., a mouse, rat, primate, or some other non-human), or other animal (e.g., Xenopus, zebrafish, or an invertebrate such as a fly or nematode). The Klotho fusion polypeptide can be administered to the organism once or as a regimen (regular or irregular). A parameter of the organism is then evaluated, e.g., an age-associated parameter. Klotho fusion polypeptides that are of interest result in a change in the parameter relative to a reference, e.g., a parameter of a control organism. Other parameters (e.g., related to toxicity, clearance, and pharmacokinetics) can also be evaluated.

The Klotho fusion polypeptide of the invention may be evaluated using an animal that has a particular disorder, e.g., a disorder described herein, e.g., an age-related disorder, a metabolic disorder. These disorders can also provide a sensitized system in which the test polypeptide's effects on physiology can be observed. Exemplary disorders include: denervation, disuse atrophy; metabolic disorders (e.g., disorder of obese and/or diabetic animals such as db/db mouse and ob/ob mouse); cerebral, liver ischemia; cisplatin/taxol/vincristine models; various tissue (xenograph) transplants; transgenic bone models; pain syndromes (include inflammatory and neuropathic disorders); Paraquat, genotoxic, and oxidative stress models; and tumor I models.

For measuring an age-related disorder, the animal model can be an animal that has an altered phenotype when calorically restricted. For example, F344 rats provide a useful assay system for evaluating a Klotho fusion polypeptide. When calorically restricted, F344 rats have a 0 to 10% incidence of nephropathy. However, when fed ad libitum, they have a 60 to 100% incidence of nephropathy.

To evaluate a Klotho fusion polypeptide of the invention, it is administered to the animal (e.g., an F344 rat or other suitable animal) and a parameter of the animal is evaluated, e.g., after a period of time. The animal can be fed ad libitum or normally (e.g., not under caloric restriction, although some parameters can be evaluated under such conditions). Typically, a cohort of such animals is used for the assay. Generally, a test polypeptide can be indicated as favorably altering lifespan regulation in the animal if the test polypeptide affects the parameter in the direction of the phenotype of a similar animal subject to caloric restriction. Such test polypeptides may cause at least some of the lifespan regulatory effects of caloric restriction, e.g., a subset of such effects, without having to deprive the organism of caloric intake.

The parameter to be tested may be an age-associated or disease associated parameter, e.g., a symptom of the disorder associated with the animal model. For example, the test polypeptide can be administered to a SH Rat, and blood pressure is monitored. A test polypeptide that is favorably indicated can cause an amelioration of the symptom relative to a similar reference animal not treated with the polypeptide. Other parameters relevant to a disorder or to aging can include: antioxidant levels (e.g. antioxidant enzyme levels or activity), stress resistance (e.g., paraquat resistance), core body temperature, glucose levels, insulin levels, thyroid-stimulating hormone levels, prolactin levels, and leutinizing hormone levels.

To measure the effectiveness of the polypeptides of the invention for treating an age-related disorder, an animal having decreased Klotho expression may be used, e.g., mouse with a mutant Klotho; See Kuroo, et al. Nature, 390; 45 (1997) and U.S. Pub. No. 2003/0119910, both of which are herein incorporated by reference in their entirety. For example, the test polypeptide is administered to the mutant mouse and age-related parameters are monitored. A test polypeptide that is favorably indicated can cause an amelioration of the symptom relative to a similar reference animal not treated with the polypeptide. A parameter relevant to a metabolic disorder or to aging can be assessed by measurement of body weight, examination on the acquisition of reproductive ability, measurement of blood sugar level, observation of life span, observation of skin, observation of motor functions such as walking, and the like. The assessment can also be made by measurement of thymus weight, observation of the size of calcified nodules formed on the inner surface of thoracic cavity, and the like. Further, quantitative determination of mRNA for the Klotho gene or Klotho protein is also useful for the assessment.

Still other in vivo models and organismal assays include evaluating an animal for a metabolic parameter, e.g., a parameter relevant to an insulin disorder, type II diabetes. Exemplary metabolic parameters include: glucose concentration, insulin concentration, and insulin sensitivity.

Another exemplary system features tumors, e.g., in an animal model. The tumors can be spontaneous or induced. For example, the tumors can be developed from cells that have a variety of genetic constitutions, e.g., they can be p53+ or p53−. It is also possible to use organisms that an autoimmune disorder, e.g., an NZB mouse, which is predisposed to SLE. To evaluate features of bone disease, it is possible, for example, to use an animal that has an ovariectomy as a model, e.g., for osteoporosis. Similarly, for joint disease, the model can be based on adjuvant arthritis (e.g., mice can be immunized with cartilage proteoglycans, high mobility group proteins, streptococcal cell wall material, or collagens); for kidney disease, kd/kd mice can be used. Animal models of cognition, particularly learning and memory are also available. Animal models of diabetes and its complications are also available, e.g., the streptozotocin model. Canine models can be used, for example, for evaluating stroke and ischemia.

In assessing whether a test polypeptide is capable of altering life span regulation, a number of age-associated parameters or biomarkers can be monitored or evaluated. Exemplary age associated parameters include: (i) lifespan of the cell or the organism; (ii) presence or abundance of a gene transcript or gene product in the cell or organism that has a biological age dependent expression pattern; (iii) resistance of the cell or organism to stress; (iv) one so or more metabolic parameters of the cell or organism (exemplary parameters include circulating insulin levels, blood glucose levels; fat content; core body temperature and so forth); (v) proliferative capacity of the cell or a set of cells present in the organism; and (vi) physical appearance or behavior of the cell or organism.

The term “average lifespan” refers to the average of the age of death of a cohort of organisms. In some cases, the “average lifespan” is assessed using a cohort of genetically identical organisms under controlled environmental conditions. Deaths due to mishap are discarded. Where average lifespan cannot be determined (e.g., for humans) under controlled environmental conditions, reliable statistical information (e.g., from actuarial tables) for a sufficiently large population can be used as the average lifespan.

Characterization of molecular differences between two such organisms, e.g., one reference organism and one organism treated with a Klotho fusion polypeptide can reveal a difference in the physiological state of the organisms. The reference organism and the treated organism are typically the same chronological age. The term “chronological age” as used herein refers to time elapsed since a preselected event, such as conception, a defined embryological or fetal stage, or, more preferably, birth. A variety of criteria can be used to determine whether organisms are of the “same” chronological age for the comparative analysis. Typically, the degree of accuracy required is a function of the average lifespan of a wildtype organism. For example, for the nematode C. elegans, for which the laboratory wildtype strain N2 lives an to average of about 16 days under some controlled conditions, organisms of the same age may have lived for the same number of days. For mice, organism of the same age may have lived for the same number of weeks or months; for primates or humans, the same number of years (or within 2, 3, or 5 years); and so forth. Generally, organisms of the same chronological age may have lived for an amount of time within 15, 10, 5, 3, 2 or 1% of the average lifespan of a wildtype organism of that species. Preferably, the organisms are adult organisms, e.g., the organisms have lived for at least an amount of time in which the average wildtype organism has matured to an age at which it is competent to reproduce.

The organismal screening assay can be performed before the organisms exhibit overt physical features of aging. For example, the organisms may be adults that have lived only 10, 30, 40, 50, 60, or 70% of the average lifespan of a wildtype organism of the same species. Age-associated changes in metabolism, immune competence, and chromosomal structure have been reported. Any of these changes can be evaluated, either in a test subject (e.g., for an organism based assay), or for a patient (e.g., prior, during or after treatment with a therapeutic described herein.

A marker associated with caloric restriction can also be evaluated in a subject organism of a screening assay (or a treated subject). Although these markers may not be age-associated, they may be indicative of a physiological state that is altered when the Klotho pathway is modulated. The marker can be an mRNA or protein whose abundance changes in calorically restricted animals. WOO1/12851 and U.S. Pat. No. 6,406,853 describe exemplary markers. Cellular models derived from cells of an animal described herein or analogous to an animal model described herein can be used for a cell-based assay.

Models for evaluating the effect of a test polypeptide on muscle atrophy include: 1) rat medial gastrocnemius muscle mass loss resulting from denervation, e.g., by severing the right sciatic nerve at mid-thigh; 2) rat medial gastrocnemius muscle mass loss resulting from immobilization, e.g., by fixed the right ankle joint at 90 degrees of flexion; 3) rat medial gastrocnemius muscle mass loss resulting from hind limb suspension; (see, e.g., U.S. 2003-0129686); 4) skeletal muscle atrophy resulting from treatment with the cachectic cytokine, interleukin-1 (IL-1) (R. N. Cooney, S. R. Kimball, T. C. Vary, Shock 7, 1-16 (1997)); and 5) skeletal muscle atrophy resulting from treatment with the glucocorticoid, dexamethasone (A. L. Goldberg, J Biol Chem 244, 3223-9 (1969).)

Exemplary animal models for AMD include: laser-induced mouse model simulating exudative (wet) macular degeneration Bora et al., Proc. Natl. Acad. Sci. USA., 100:2679-84 (2003); a transgenic mouse expressing a mutated form of cathepsin D resulting in features associated with the “geographic atrophy” form of AMD (Rakoczy et al., Am. J Pathol., 161:1515-24 (2002)); and a transgenic mouse over expressing VEGF in the retinal pigment epithelium resulting in CNV. Schwesinger et al., Am. J. Pathol. 158:1161-72 (2001).

Exemplary animal models of Parkinson's disease include primates rendered Parkinsonian by treatment with the dopaminergic neurotoxin 1-methyl-4 phenyl 1,2,3,6-tetrahydropyridine (MPTP) (see, e.g., U.S. Patent Publication No. 20030055231 and Wichmann et al., Ann. N.Y. Acad. Sci., 991:199-213 (2003); 6-hydroxydopamine-lesioned rats (e.g., Lab. Anim. Sci., 49:363-71 (1999)); and transgenic invertebrate models (e.g., Lakso et al., J. Neurochem. 86:165-72 (2003) and Link, Mech. Ageing Dev., 122:1639-49 (2001)).

Exemplary molecular models of Type II diabetes include: a transgenic mouse having defective Nkx-2.2 or Nkx-6.1; (U.S. Pat. No. 6,127,598); Zucker Diabetic Fatty fa/fa (ZDF) rat. (U.S. Pat. No. 6,569,832); and Rhesus monkeys, which spontaneously develop obesity and subsequently frequently progress to overt type 2 diabetes (Hotta et al., Diabetes, 50:1126-33 (2001); and a transgenic mouse with a dominant-negative IGF-I receptor (KR-IGF-IR) having Type 2 diabetes-like insulin resistance.

Exemplary animal and cellular models for neuropathy include: vincristine induced sensory-motor neuropathy in mice (U.S. Pat. No. 5,420,112) or rabbits (Ogawa et al., Neurotoxicology, 21:501-11 (2000)); a streptozotocin (STZ)-diabetic rat for study of autonomic neuropathy (Schmidt et al., Am. J. Pathol., 163:21-8 (2003)); and a progressive motor neuropathy (pmn) mouse (Martinet al., Genomics, 75:9-16 (2001)).

Exemplary animal models of hyperphosphatemia or tumoral calcinosis include Klotho knockout mice and FGF23 knockout mice (Yoshida et al., Endocrinology 143:683-689 (2002)).

Exemplary animal models of chronic renal disease or chronic renal failure include COL4A3+/−mice (Beirowski et al., J. Am. Soc. Nephrol. 17:1986-1994 (2006)).

Exemplary animal models of cancer include the transplantation or implantation of cancer cells or tissue into nude mice, as is known in the art (Giovanella et al., Adv. Cancer Res. 44:69-120 (1985)). For example, animal models of breast cancer include nude mice transplanted or implanted with breast cancer cells or tissue (e.g., Yue et al., Cancer Res. 54:5092-5095 (1994); Glinsky et al., Cancer Res. 56:5319-5324 (1996); Visonneau Am. J. Path. 152:1299-1311 (1998)).

The compositions can be administered to a subject, e.g., an adult subject, particularly a healthy adult subject or a subject having an age-related disease. In the latter case, the method can include evaluating a subject, e.g., to characterize a symptom of an age-related disease or other disease marker, and thereby identifying a subject as having a neurodegenerative disease, e.g., Alzheimer's or an age-related disease or being pre-disposed to such a disease.

### Skeletal Muscle Atrophy

Methods of the invention which provide administering the Klotho fusion polypeptide to an individual can be used to treat skeletal muscle atrophy. Muscle atrophy includes numerous neuromuscular, metabolic, immunological and neurological disorders and diseases as well as starvation, nutritional deficiency, metabolic stress, diabetes, aging, muscular dystrophy, or myopathy. Muscle atrophy occurs during the aging process. Muscle atrophy also results from reduced use or disuse of the muscle. Symptoms include a decline in skeletal muscle tissue mass. In human males, muscle mass declines by one-third between the ages of 50 and 80. Some molecular features of muscle atrophy include the upregulation of ubiquitin ligases, and the loss of myofibrillar proteins (Furuno et al., J. Bioi. Chern., 265:8550-8557, 1990). The breakdown of these proteins can be followed, e.g., by measuring 3-methyl-histidine production, which is a specific constituent of actin, and in certain muscles of myosin (Goodman, Biochem. J. 241:121-12, 1987 and Lowell, et al., Metabolism, 35:1121-112, 1986; Stein and Schluter, Am. J. Physiol. Endocrinol. Metab. 272: E688-E696, 1997). Release of creatine kinase (a cell damage marker) (Jackson, et al., Neurology, 41: 101 104, 1991) can also be indicative.

### Non-Insulin-Dependent Diabetes

Methods of the invention which provide administering the Klotho fusion polypeptide to an individual can be used to treat Non-insulin-dependent Diabetes. Non-insulin-dependent Diabetes is also called “adult onset” diabetes and Type 2 diabetes. Type 2 diabetes also includes “non-obese type 2” and “obese type 2.” Type II diabetes can be characterized by (1) reduced pancreatic-beta-islet-cell secretion of insulin such that less than necessary amounts of insulin are produced to keep blood glucose levels in balance and/or (2) “insulin resistance,” wherein the body fails to respond normally to insulin. (U.S. Pat. No. 5,266,561 and U.S. Pat. No. 6,518,069). For example, glucose-stimulated insulin levels typically fail to rise above 4.0 nmol/L. (U.S. Pat. No. 5,266,561). Exemplary symptoms ofType II diabetes include: hyperglycemia while fasting (U.S. Pat. No. 5,266,561); fatigue; excessive thirst; frequent urination; blurred vision; and an increased rate of infections. Molecular indications of Type II diabetes include islet amyloid deposition in the pancreases.

### Neuropathy

Neuropathy can include a central and/or peripheral nerve dysfunction caused by systemic disease, hereditary condition or toxic agent affecting motor, sensory, sensorimotor or autonomic nerves. (see, e.g., US Patent Application No. 20030013771). Symptoms can vary depending upon the cause of the nerve damage and the particular types of nerves affected. For example, symptoms of motor neuropathy include clumsiness in performing physical tasks or as muscular weakness, exhaustion after minor exertion, difficulty in standing or walking and attenuation or absence of a neuromuscular reflex. (U.S. Patent Application No. 20030013771) symptoms of autonomic neuropathy include constipation, cardiac irregularities and attenuation of the postural hypotensive reflex. (U.S. Patent Application No. 20030013771), symptoms of sensory neuropathy include pain and numbness; tingling in the hands, legs or feet; and extreme sensitivity to touch, and symptoms of retinopathy include blurred vision, sudden loss of vision, black spots, and flashing lights.

### Alzheimer's Disease

Methods of the invention which provide administering the Klotho fusion polypeptide to an individual can be used to treat Alzheimer's Disease (AD). Alzheimer's Disease is a complex neurodegenerative disease that results in the irreversible loss of neurons. It provides merely one example of a neurodegenerative disease that is also an age-related condition. Clinical hallmarks of Alzheimer's Disease include progressive impairment in memory, judgment, orientation to physical surroundings, and language. Neuropathological hallmarks of AD include region-specific neuronal loss, amyloid plaques, and neurofibrillary tangles. Amyloid plaques are extracellular plaques containing the amyloid peptide (also known as Ap, or Ap42), which is a cleavage product of the, 8-amyloid precursor protein (also known as APP). Neurofibrillary tangles are insoluble intracellular aggregates composed of filaments of the abnormally hyperphosphorylated microtubule-associated protein, taut Amyloid plaques and neurofibrillary tangles may contribute to secondary events that lead to neuronal loss by apoptosis (Clark and Karlawish, Ann. Intern. Med. 138(5):400-410 (2003). For example, p-amyloid induces caspase-2-dependent apoptosis in cultured neurons (Troy et al. J Neurosci. 20(4):1386-1392). The deposition of plaques in viva may trigger apoptosis of proximal neurons in a similar manner.

A variety of criteria, including genetic, biochemical, physiological, and cognitive criteria, can be used to evaluate AD in a subject. Symptoms and diagnosis of AD are known to medical practitioners. Some exemplary symptoms and markers of AD are presented below. Information about these indications and other indications known to be associated with AD can be used as an “AD-related parameter.” An AD related parameter can include qualitative or quantitative information. An example of quantitative information is a numerical value of one or more dimensions, e.g., a concentration of a protein or a tomographic map. Qualitative information can include an assessment, e.g., a physician's comments or a binary (“yes”/“no”) and so forth. An AD-related parameter includes information that indicates that the subject is not diagnosed with AD or does not have a particular indication of AD, e.g., a cognitive test result that is not typical of AD or a genetic APOE polymorphism not associated with AD.

Progressive cognitive impairment is a hallmark of AD. This impairment can present as decline in memory, judgment, decision making, orientation to physical surroundings, and language (Nussbaum and Ellis, New Eng J. Med. 348(14):1356 35 1364 (2003)). Exclusion of other forms of dementia can assist in making a diagnosis of AD. Neuronal death leads to progressive cerebral atrophy in AD patients. Imaging techniques (e.g., magnetic resonance imaging, or computer assisted tomography) can be used to detect AD-associated lesions in the brain and/or brain atrophy.

AD patients may exhibit biochemical abnormalities that result from the pathology of the disease. For example, levels often protein in the cerebrospinal fluid is elevated in AD patients (Andreasen, N. et al. Arch Neural. 58:349-350 (2001)).

Levels of amyloid beta 42 (A,B42) peptide can be reduced in CSF of AD patients. Levels of Ap42 can be increased in the plasma of AD patients (Ertekein-Taner, N., et al. Science 290:2303 2304 (2000)). Techniques to detect biochemical abnormalities in a sample from a subject include cellular, immunological, and other biological methods known in the art. For general guidance, see, e.g., techniques described in Sambrook & Russell, Molecular Cloning: A Laboratory Manual, 3r Edition, Cold Spring Harbor Laboratory, N.Y. (2001), Ausubel et al., Current Protocols in Molecular Biology (Greene Publishing Associates and Wiley Interscience, N.Y. (1989), (Harrow, E. and Lane, D. (1988) Antibodies: A Laboratory Manual, Cold Spring Harbor Laboratory Press, Cold Spring Harbor, N.Y.), and updated editions thereof.

For example, antibodies, other immunoglobulins, and other specific binding ligands can be used to detect a biomolecule, e.g., a protein or other antigen associated with AD. For example, one or more specific antibodies can be used to probe a sample. Various formats are possible, e.g., ELISAs, fluorescence-based assays, Western blots, and protein arrays. Methods of producing polypeptide arrays are described in the art, e.g., in De Wildt et al. (2000). Nature Biotech. 18, 989-994; Lucking et al. (1999). Anal. Biochem. 270, 103-111; Ge, H. (2000). Nucleic Acids Res. 28, e3, I-VII; MacBeath, G., and Schreiber, S. L. (2000). Science 289, 1760 to 1 763; and WO 99/5 1 773A1.

In one assay, a non-human animal model of AD (e.g., a mouse model) is used, e.g., to evaluate a polypeptide or a therapeutic regimen. For example, U.S. Pat. No. 6,509,515 describes one such model animal which is naturally able to be used with learning and memory tests. The animal expresses an amyloid precursor protein (APP) sequence at a level in brain tissues such that the animal develops a progressive neurologic disorder within a short period of time from birth, generally within a year from birth, preferably within 2 to 6 months, from birth. The APP protein sequence is introduced into the animal, or an ancestor of the animal, at an embryonic stage, preferably the one cell, or fertilized oocyte, stage, and generally not later than about the 8-cell stage. The zygote or embryo is then developed to term in a pseudo-pregnant as foster female. The amyloid precursor protein genes are introduced into an animal embryo so as to be chromosomally incorporated in a state which results in super endogenous expression of the amyloid precursor protein and the development of a progressive necrologic disease in the cortico-limbic areas of the brain, areas of the brain which are prominently affected in progressive necrologic disease states such as AD. The gliosis and clinical manifestations in affected transgenic animals model necrologic disease. The progressive aspects of the neurologic disease are characterized by diminished exploratory and/or locomotor behavior and diminished deoxyglucose uptake/utilization and hypertrophic gliosis in the cortico-limbic regions of the brain. Further, the changes that are seen are similar to those that are seen in some aging animals. Other animal models are also described in U.S. Pat. Nos. 5,387,742; 5,877,399; 6,358,752; and 6, 187,992.

### Parkinson's Disease

Methods of the invention which provide administering the Klotho fusion polypeptide to an individual can be used to treat Parkinson's Disease. Parkinson's disease includes neurodegeneration of dopaminergic neurons in the substantia nigra resulting in the degeneration of the nigrostriatal dopamine system that regulates motor function. This pathology, in turn, leads to motor dysfunctions. (see, e.g., and Lotharius et al., Nat. Rev. Neurosci., 3:932-42 (2002)). Exemplary motor symptoms include: akinesia, stooped posture, gait difficulty, postural instability, catalepsy, muscle rigidity, and tremor. Exemplary non-motor symptoms include: depression, lack of motivation, passivity, dementia and gastrointestinal dysfunction (see, e. g., Fahn, Ann. N.Y. Acad. Sci., 991:1-14 (2003) and Pfeiffer, Lancet Neural., 2:107-16 (2003)) Parkinson's has been observed in 0.5 to 1 percent of persons 65 to 69 years of age and 1 to 3 percent among persons 80 years of age and older. (see, e.g., Nussbaum et al., N. Engl. J. Med., 348:1356-64 (2003)). Molecular markers of Parkinson's disease include reduction in aromatic L amino acid decarboxylase (AADC) (see, e.g., US App. No. 20020172664); and loss of dopamine content in the nigrostriatal neurons (see, e.g., Fahn, Ann. N.Y. Acad. Sci., 991:1-14 (2003) and Lotharius et al., Nat. Rev. Neurosci., 3:932-42 (2002)). In some familial cases, PD is linked to mutations in single genes encoding alpha-synuclein and parkin (an E3 ubiquitin ligase) proteins. (e.g., Riess et al., J. Neurol. 250 Suppl 1:13 10 (2003) and Nussbaum et al., N. Engl. J. Med., 348:1356-64 (2003)). A missense mutation in a neuron-specific C-terminal ubiquitin hydrolase gene is also associated with Parkinson's. (e.g., Nussbaum et al., N. Engl. J. Med., 348:1356-64 (2003))

### Huntington's Disease

Methods of the invention which provide administering the Klotho fusion polypeptide to an individual can be used to treat Huntington's Disease. Methods for evaluating the efficacy and/or determining the effective dose of a Klotho fusion polypeptide on Huntington's Disease include organismal based assays, e.g., using a mammal (e.g., a mouse, rat, primate, or some other non-human), or other animal (e.g., Xenopus, zebrafish, or an invertebrate such as a fly or nematode). A number of animal model system for Huntington's disease are available. See, e.g., Brouillet, Functional Neurology 15(4): 239-251 (2000); Ona et al. Nature 399: 263-267 (1999), Bates et al. Hum Mol Genet. 6(10): 1633-7 (1997); Hansson et al. J. of Neurochemistry 78: 694-703; and Rubinsztein, D. C., Trends in Genetics, Vol. IS, No. 4, pp. 202-209 (a review on various animal and non-human models ofHD).

An example of such an animal model is the transgenic mouse strain is the R6/2 line (Mangiarini et al. Cell 87: 493-506 (1996)). The R6/2 mice are transgenic Huntington's disease mice, which over-express exon 1 of the human HD gene (under the control of the endogenous promoter). The exon 1 of the R6/2 human HD gene has an expanded CAG/polyglutamine repeat lengths (150 CAG repeats on average). These mice develop a progressive, ultimately fatal neurological disease with many features of human Huntington's disease. Abnormal aggregates, constituted in part by the N terminal part of Huntingtin (encoded by HD exon 1), are observed in R6/2 mice, both 45 in the cytoplasm and nuclei of cells (Davies et al. Cell 90: 537-548 (1997)). For example, the human Huntingtin protein in the transgenic animal is encoded by a gene that includes at least 55 CAG repeats and more preferably about 150 CAG repeats. These transgenic animals can develop a Huntington's disease-like phenotype.

These transgenic mice are characterized by reduced weight gain, reduced lifespan and motor impairment characterized by abnormal gait, resting tremor, hindlimb clasping and hyperactivity from 8 to 10 weeks after birth (for example the R6/2 strain; see Mangiarini et al. Cell 87: 493-506 (1996)). The phenotype worsens progressively toward hypokinesia. The brains of these transgenic mice also demonstrate neurochemical and histological abnormalities, such as changes in neurotransmitter receptors (glutamate, dopaminergic), decreased concentration of N-acetylaspartate (a marker of neuronal integrity) and reduced striatum and brain size. Accordingly, evaluating can include assessing parameters related to neurotransmitter levels, neurotransmitter receptor levels, brain size and striatum size. In addition, abnormal aggregates containing the transgenic part of or full-length human Huntingtin protein are present in the brain tissue of these animals (e.g., the R6/2 transgenic mouse strain). See, e.g., Mangiarini et al. Cell 87: 493-506 (1996), Davies et al. Cell 90: 537-548 (1997), Brouillet, Functional Neurology 15(4): 239-251 (2000) and Chaetal. Proc. Natl. Acad. Sci. USA 95: 6480-6485 (1998).

To test the effect of the test polypeptide or known polypeptide described in the application in an animal model, different concentrations of test polypeptide are administered to the transgenic animal, for example by injecting the test polypeptide into circulation of the animal. A Huntington's disease-like symptom may be evaluated in the animal. The progression of the Huntington's disease-like symptoms, e.g., as described above for the mouse model, is then monitored to determine whether treatment with the test polypeptide results in reduction or delay of symptoms. In another assay, disaggregation of the Huntingtin protein aggregates in these animals is monitored. The animal can then be sacrificed and brain slices are obtained. The brain slices are then analyzed for the presence of aggregates containing the transgenic human Huntingtin protein, a portion thereof, or a fusion protein comprising human Huntingtin protein, or a portion thereof This analysis can includes, for example, staining the slices of brain tissue with anti-Huntingtin antibody and adding a secondary antibody conjugated with FITC which recognizes the anti-Huntington's antibody (e.g., the anti-Huntingtin antibody is mouse anti-human antibody and the secondary antibody is specific for human antibody) and visualizing the protein aggregates by fluorescent microscopy.

A variety of methods are available to evaluate and/or monitor Huntington's disease. A variety of clinical symptoms and indicia for the disease are known. Huntington's disease causes a movement disorder, psychiatric difficulties and cognitive changes. The degree, age of onset, and manifestation of these symptoms can vary. The movement disorder can include quick, random, dance-like movements called chorea.

Exemplary motor evaluations include: ocular pursuit, saccade initiation, saccade velocity, dysarthria, tongue protrusion, finger tap ability, pronate/supinate, a lo fist-hand-palm sequence, rigidity of arms, bradykinesia, maximal dystonia (trunk, upper and lower extremities), maximal chorea (e.g., trunk, face, upper and lower extremities), gait, tandem walking, and retropulsion. An exemplary treatment can cause a change in the Total Motor Score 4 (TMS-4), a subscale of the UHDRS, e.g., over a one-year period.

### Cancer

Methods of the invention which provide administering the Klotho fusion polypeptide to an individual can be used to treat cancer. Cancer includes any disease that is caused by or results in inappropriately high levels of cell division, inappropriately low levels of apoptosis, or both. Examples of cancers include, without limitation, leukemias (e. g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

All patents, patent applications, and published references cited herein are hereby incorporated by reference in their entirety. While this invention has been particularly shown and described with references to embodiments thereof, it will be understood by those skilled in the art that various changes in form and details may be made therein without departing from the scope of the invention encompassed by the appended claims.

### 5. EXAMPLES

### Example 1. Expression and Purification of Klotho Fusion Polypeptides

Expression of the Klotho Fusion Polypeptide

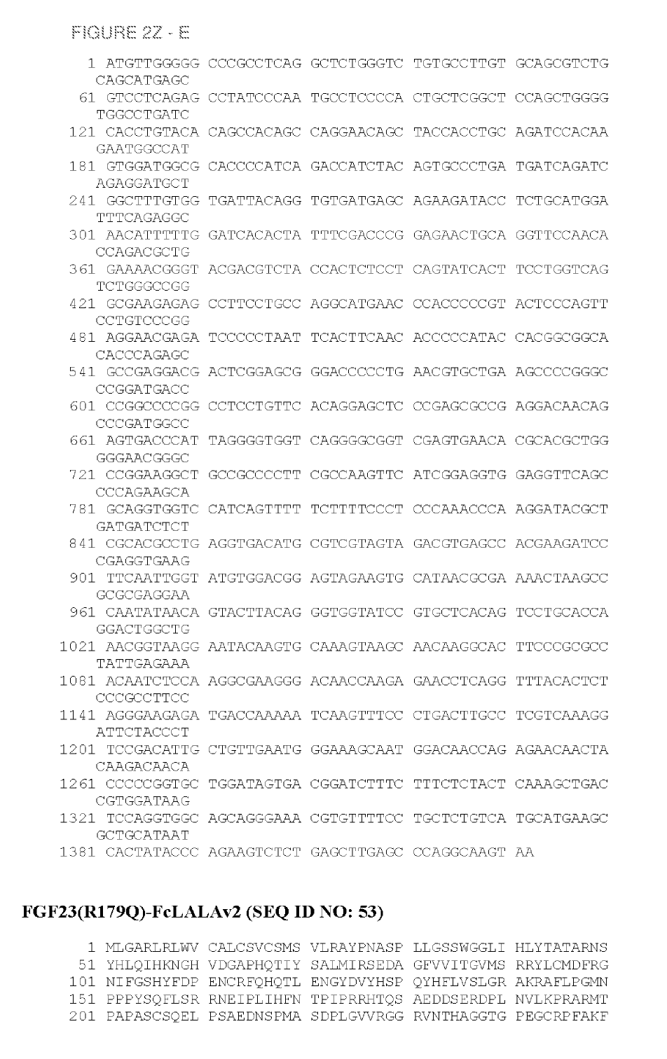

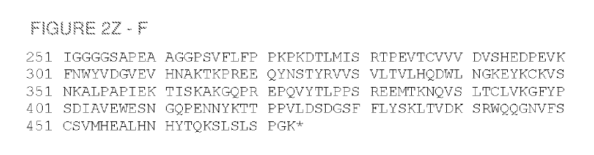

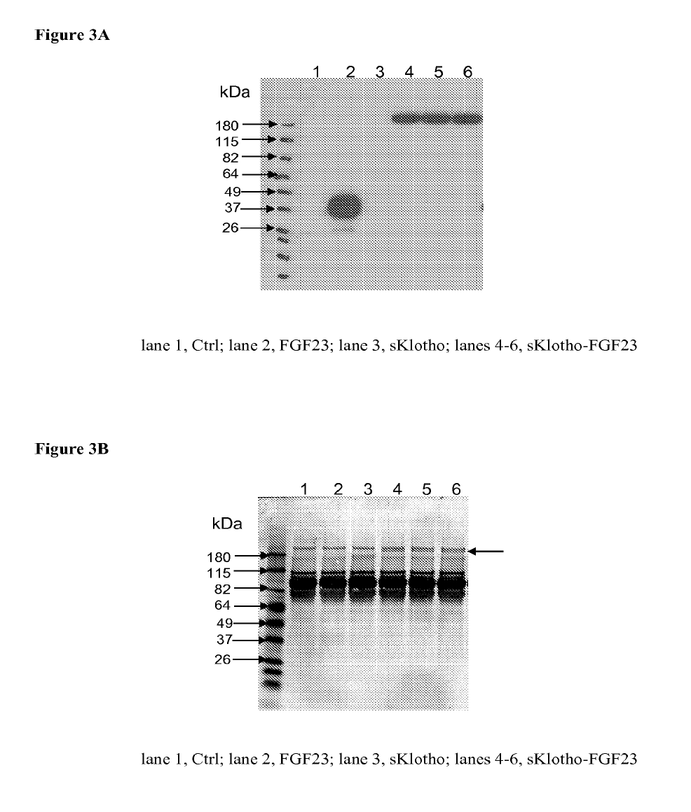

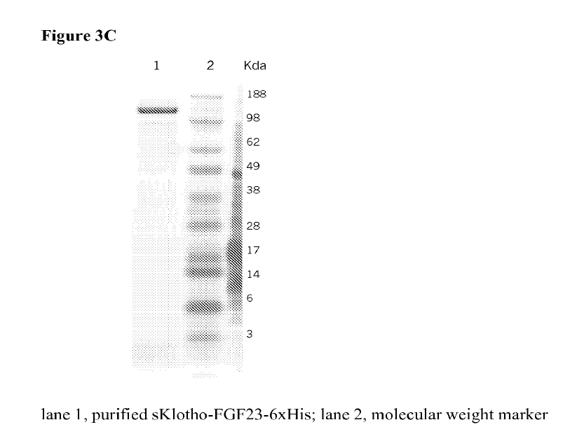

The polypeptides of the invention were made by transiently transfecting HEK293T cells with an expression vector encoding a Klotho fusion polypeptide having the extracellular domain of alpha Klotho and the FGF23 (R179Q) variant. Conditioned media containing expressed polypeptides were generated by transient transfection of the respective expression plasmids for Klotho, FGF23, and the Klotho-FGF23(R179Q) fusion protein. The transfections were performed in 6-well plates using Lipofectamine 2000 (Invitrogen, Cat#11668-019). Five hours after transfection, the transfection mix was replaced with 3 ml DMEM plus 1% FBS. Conditioned media were collected 72 hours after the addition of 3 ml DMEM plus 1% FBS. Samples of conditioned medium from various transiently transfected HEK293T cells were separated by SDS-polyacrylamide gel electrophoresis (SDS-PAGE) and analyzed by Western blot (FIG. 3A) or stained with Coomassie blue (FIG. 3B).

SDS-polyacrylamide gel electrophoresis was performed on various samples (lane 1, Control; lane 2, FGF23; lane 3, sKlotho; lanes 4-6, sKlotho-FGF23). Coomassie blue staining revealed the expression of a high, >180 kDa band (FIG. 3B, indicated by arrow on the right) that was not present in lanes 1-3, which contained samples that had not been transfected with the vector encoding the Klotho fusion polypeptide. The quality of the Klotho fusion polypeptide secreted into the media was evaluated by Western blot (FIG. 3A). An anti-FGF23 rat monoclonal IgG2A (R&D Systems, Cat# MAB26291) was used as the primary antibody to detect the Klotho fusion polypeptides by Western blot. The Western blot confirmed that the additional bands observed in the Coomassie stained gels were Klotho fusion polypeptides. The Western blot confirmed that the Klotho fusion polypeptides had the expected molecular weight for the Klotho fusion polypeptide. This analysis shows the expression of the Klotho-FGF23(R179Q) fusion protein.

Purification of the Klotho Fusion Polypeptide

The polypeptides of the invention were purified from conditioned media from a culture of HEK293T cells transiently transfected with an expression vector encoding a Klotho fusion polypeptide having the extracellular domain of alpha Klotho and the FGF23 R179Q variant. To generate conditioned medium, an expression vector encoding sKlotho-FGF23-6×His was transfected (500 μg DNA in 18 ml of OptiMEM 1 (GIBCO, Cat #11058) mixed with 18 ml of 2 g/ml polyethlinimine (PEl) into HEK293 cells grown in suspension in expression medium (464 ml of HEK293T cells at 10⁶ cells/ml in Freestype 293 expression medium (GIBCO, Cat #12338)). After transfection, the culture was allowed to grow (120 hours; 37° C. in a 5% CO₂ incubator; shaking at 125 rpm). At the end of incubation, conditioned medium was harvested by centrifugation (1000 rpm for five minutes). The conditioned medium was then applied to a nickel-agarose column. The sKlotho-FGF23-6×His bound tightly to the column and was eluted with 50 mM imidazole. The resulting purified material was then dialyzed in PBS to remove imidazole. A sample of the purified sKlotho-FGF23-6×His was separated by SDS-PAGE (lane 1, purified sKlotho-FGF23-6×His; lane 2, molecular weight marker) and analyzed by staining with Coomassie blue (FIG. 3C). The stained SDS-PAGE gel confirmed that the purified sKlotho-FGF23-6×His had the expected molecular weight. The inability to detect bands corresponding to proteins other than full-length sKlotho-FGF23-6×His in the lane loaded with the purified material also showed that the sKlotho-FGF23-6×His was purified.

### Example 2. In Vitro Assay Assessing the Activity of the Klotho Fusion Polypeptide

Egr-1-luciferase

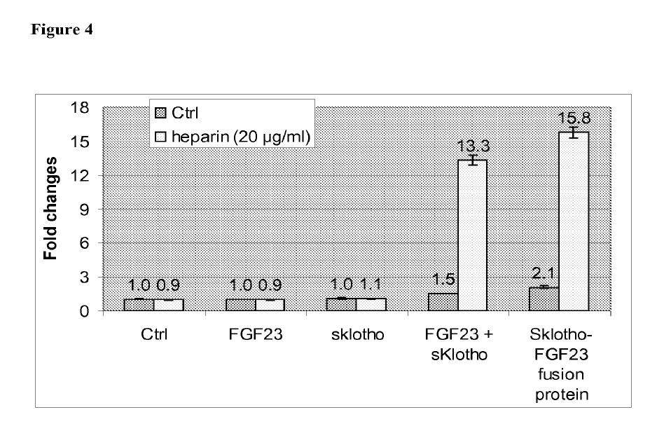

The biological activity of the expressed alpha Klotho fusion polypeptide was tested in Egr-1-luciferase reporter assays. Binding of the Klotho fusion polypeptide to the FGF23 receptor resulted in the downstream activation of Egr-1 and the expression of a luciferase reporter regulated by the Egr-1 promoter. The Egr-1-luciferase reporter gene was constructed based on that reported by Urakawa et al. (Nature, 2006, Vol 444, 770-774). HEK293T cells seeded in 48-well poly-D-lysine plate were transfected with the Egr-1-luciferase reporter gene together with a transfection normalization reporter gene (Renilla luciferase). Five hours after transfection of the Egr-1 luciferase reporter gene, the transfection mix was replaced with 3 ml DMEM plus 1% FBS. Conditioned media were collected 72 hours after the addition of 3 ml DMEM plus 1% FBS. Five hours later, the transfection mix was replaced with a sample to be tested for activity. In initial experiments, 50% conditioned medium (alone or containing Klotho, FGF23, Klotho and FGF23, and the Klotho-FGF23(R179Q) fusion protein) and 50% DMEM with 1% FBS in the presence or absence of 20) 1 g/ml heparin (Sigma, Cat#H8537; dissolved in DMEM as 2 mg/ml stock) were tested in the Egr-1-luciferase reporter assays (FIG. 4). Further experiments used defined quantities of the purified polypeptides (Figures SA and 5B). Cells were lysed 20 hours later in passive lysis buffer (Promega, Cat #E194A) and luciferase activities were determined using Dual-Glo Luciferase Assay System (Promega, Cat #E2940).

In initial experiments, Klotho fusion polypeptide activity was demonstrated in unfractionated conditioned medium. Using the Egr-1-luciferase reporter gene (FIG. 4) these experiments quantified the fold changes in the expression of the luciferase reporter. Conditioned medium containing a combination of FGF23 and the extracellular domain of Klotho protein activated Egr-1-luciferase, but conditioned medium containing only FGF23 or conditioned medium containing only the extracellular domain of Klotho, did not activate Egr-1-luciferase. Conditioned medium containing the fusion protein sKlotho-FGF23(R179Q) activated the Egr-1-luciferase reporter gene in contrast to conditioned media containing either FGF23 or Klotho alone. In these experiments, conditioned medium containing the fusion protein sKlotho-FGF23(R179Q) activated the Egr-1-luciferase reporter gene significantly better than conditioned medium containing a combination of FGF23 and Klotho. In the presence of heparin, the inductions by conditioned medium containing the fusion protein sKlotho-FGF23(R179Q) and the conditioned medium containing a combination of FGF23 and Klotho were significantly enhanced. Table 1 lists the relative expression of various FGF-Klotho fusion polypeptides in conditioned medium and the relative activity of the unfractionated conditioned medium corresponding to the various FGF-Klotho fusion polypeptides in Egr-1-luciferase reporter assays.

TABLE 1
Expression and Activities ofsKlotho-FGF23 fusion variants

| | sKlotho-FGF23 | | Activity in Egr-1-luc |
|---|---|---|---|
| | fusion constructs | Expression | reporter gene |
| 1 | sKlotho-FGF23 | good | yes |
| 2 | IgG sp-sKlotho-FGF23 | good | yes |
| 3 | sKL-D1-FGF23 | good | no |
| 4 | sKL-D2-FGF23 | no | n.a. |
| 5 | s(KL-D1)2-FGF23 | good | no |
| 6 | sKL-D1!D2-FGF23 | no | n.a. |
| 7 | ssKlotho(-26)-FGF23 | poor | no* |
| 8 | sKLD1-D2(692-965)-FGF23 | poor | no* |
| 9 | sKL-D1-D2(07-798)-FGF23 | poor | no* |
| 10 | FGF23-sKlotho | poor | no* |

*lack of activity may be the result of low expression

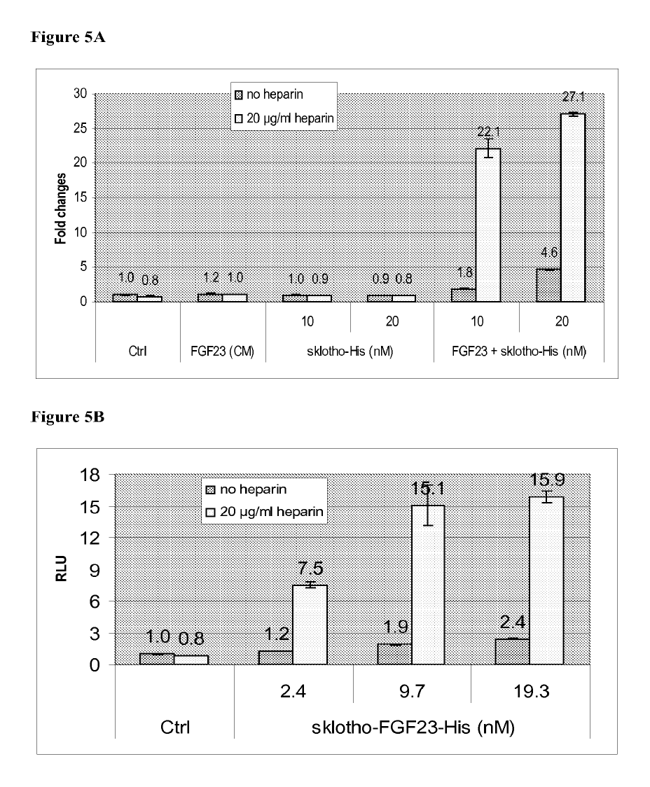

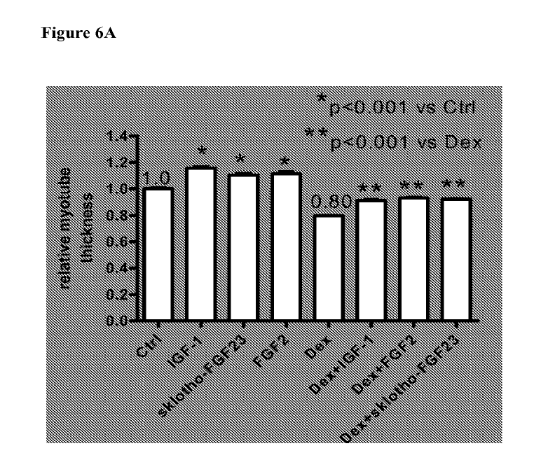

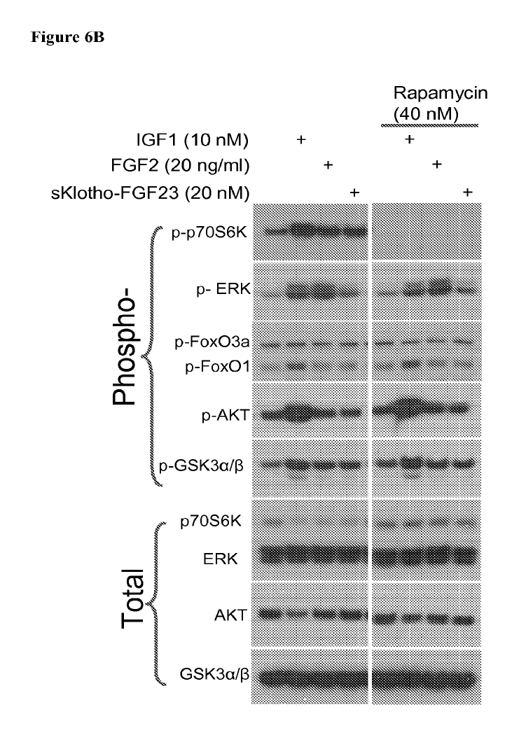

Egr-1-luciferase reporter assays were also performed using defined quantities of proteins purified from the conditioned medium, using the purification procedure as described in Example 1. Consistent with previous results using unfractionated conditioned medium containing the expressed polypeptides, treatment with a combination of purified FGF23 and sKlotho resulted in luciferase reporter activity, but treatment with purified FGF23 alone did not (Figure SA). The luciferase reporter activity from the combination of purified FGF23 and sKlotho was further dependent on the dose of purified sKlotho, and the effect could be enhanced by the presence of heparin (20 μg/ml). An effect of the sKlotho-FGF23-6×His fusion polypeptide on luciferase activity could be detected at concentrations as low as about 1.21 nM (1.2 fold change) and at least up to about 19.3 nM (2.4 fold change) in Egr-1-luciferase reporter assays (FIG. 5B). The activity of the sKlotho-FGF23-6×His fusion polypeptide on luciferase activity was significantly enhanced in the presence of heparin (20) 1 g/ml). In the presence of heparin, the effect of the sKlotho-FGF23-6×His fusion polypeptide on luciferase activity could be detected at a concentration as low as about 0.6 nM (2.0 fold change). The result showed that purified sKlotho-FGF23-6×His dose-dependently induced the EGR-1-luc reporter gene, and that treatment with sKlotho-FGF23-6×His.

### Example 3. In Vitro Assay Assessing the Effect of the Klotho Fusion Polypeptide on Muscle Cells

The biological effect of the expressed Klotho fusion polypeptide was tested on C2C12 myoblasts. Treatment of C2C12 myoblasts with IGF-1, FGF2, or sKlotho-FGF23 resulted in myotube growth and phosphorylation of signaling proteins. C2C12 myoblasts were seeded at a density of 40,000 cells/well in 6-well poly-D-lysine and fibronectin coated plates in growth medium (3 parts DMEM and 1 part F12), 10% FBS, 1% Glut; 1% P/S; 1% Linolic acid; 0.1% ITS: [insulin (10 mg/ml), transferrin (5.5 mg/ml), and selenium (5 ng/ml)]. After myoblasts reached confluence (3 days), medium was changed into differentiation medium (DMED with 2% horse serum; 1% Glut; 1% P/S).

For the myotube diameter experiments, three days after confluent media was changed into differentiation medium, cells were treated with IGF-1 (10 nM), FGF2 (20 ng/ml) or sKlotho-FGF23 (20 nM) in the absence or presence of dexamethasone (100 μM) for 24 hours in differentiation medium. At the end of treatment, cells were fixed with glutaraldehyde (5% in PBS) and multiple fluorescent images were collected. Myotube diameter was measured using the Pipeline Pilot program to determine hypertrophy or atrophy.

For the signaling protein phosphorylation, experiments, three days after confluent media was changed into differentiation medium, cells were starved for four hours with DMEM without PBS and then treated with IGF-1 (10 nM), FGF2 (20 ng/ml) or sKlotho-FGF23 (20 nM) in the absence or presence of Rapamycin (40 nM) for 30 min. Cells were lysed in RIPA buffer in the presence of protease and phosphatase inhibitors. Western blot analysis was carried out and membranes were probed with different antibodies as indicated in the figure and developed on X-ray films, which were scanned.

The results of this study showed that sKlotho-FGF23 resulted in an increase in myotube diameter compared to the control and induced C2C12 myotube hypertrophy similar to results for IGF-1 and FGF2 (Figure SA). In addition, treatment with sKlotho-FGF23, IGF-1, and FGF2 could partially reverse myotube atrophy induced by dexamethasone, based on measurements of myotube diameter. No difference was observed between sKlotho-FGF23 and FGF2 on myotube morphology (measured by thickness of the myotubes) in the absence or presence of dexamethasone. The trophic effects of sKlotho-FGF23, IGF-1, and FGF2 were statistically significant.

Consistent with the effects on C2C12 myotubes, sKlotho-FGF23 fusion protein signaling led to the phosphorylation of p70S6K and ERK, but not AKT or FoxO, in C2C12 myotubes (Figure SB). The effect of sKlotho-FGF23 on signaling was similar to that of FGF2, but was distinct from that of iGF-1. The extent of ERK phosphorylation by sKlotho-FGF23 was observed to be less than that of iGF-1 or FGF2. The phosphorylation of p70S6K by sKlotho-FGF23 was rapamycin sensitive. In the experiments involving C2C12 cells, heparin was not required to activate signaling. These results show that a sKlotho-FGF23 fusion polypeptide activated signaling in C2C12 myotubes.

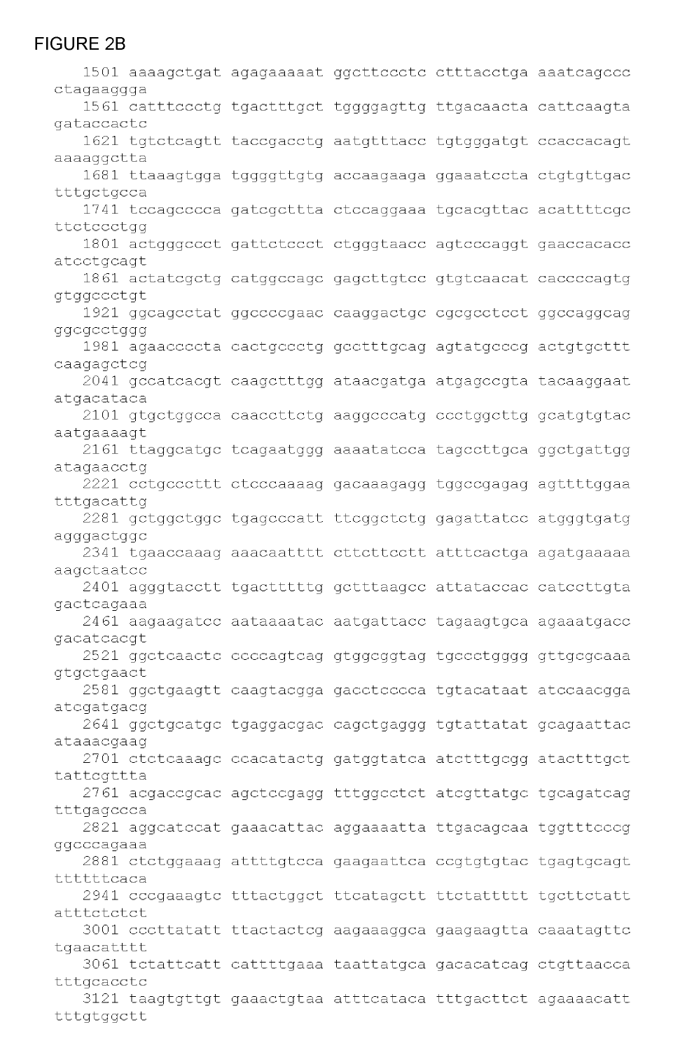

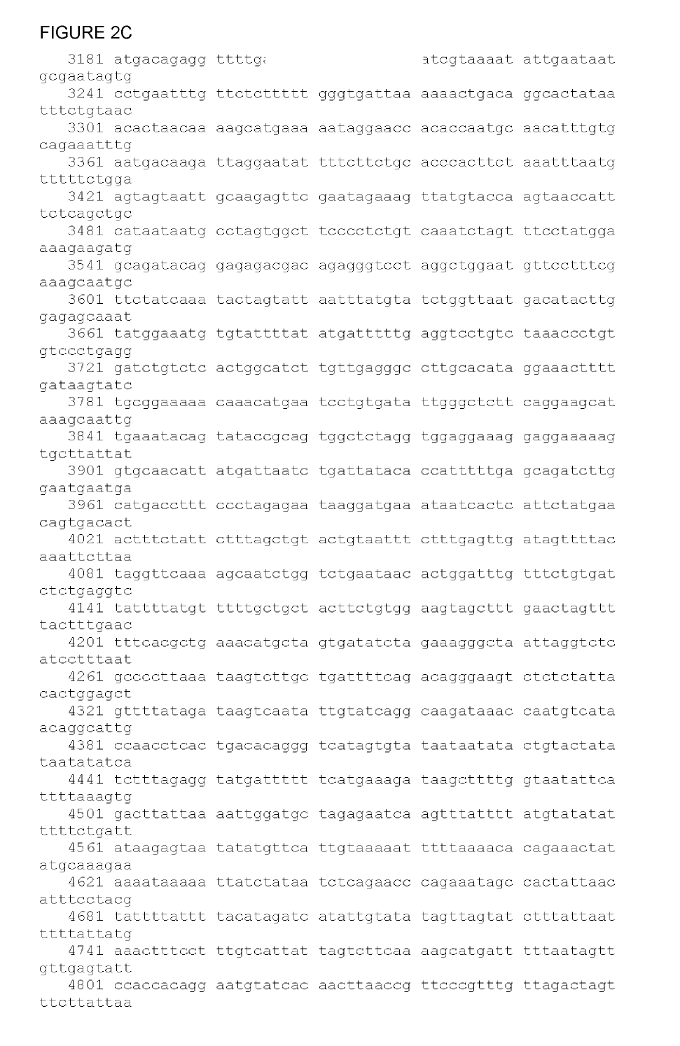

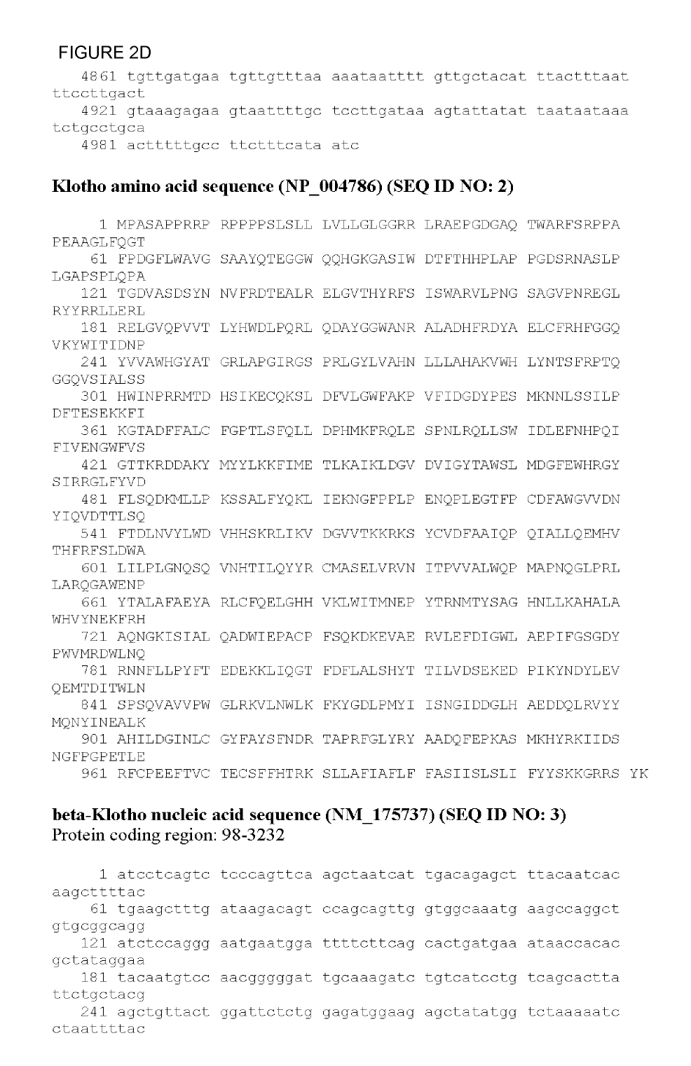

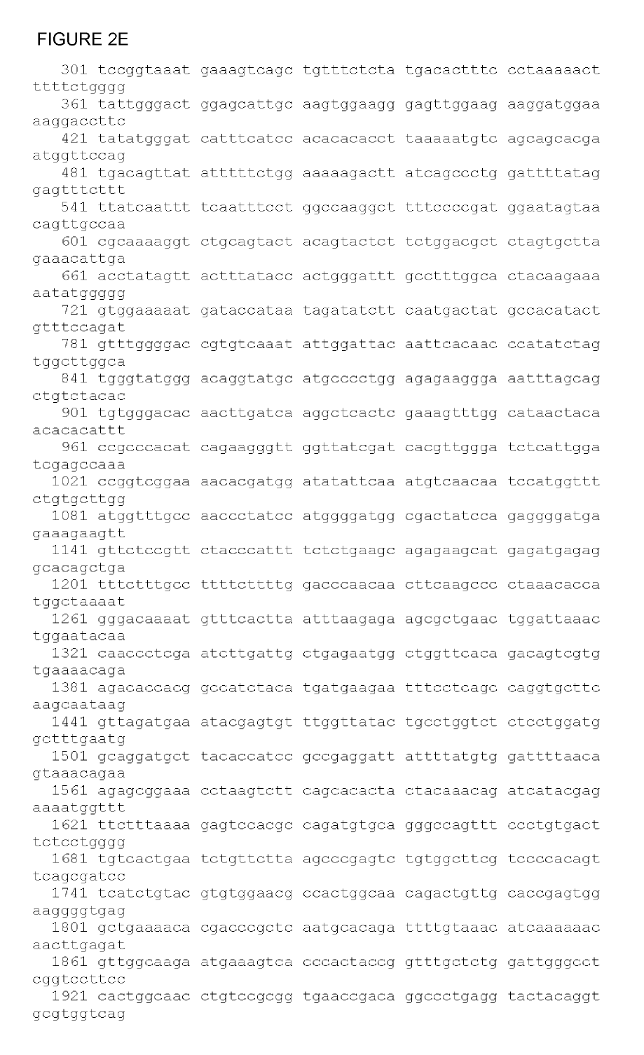

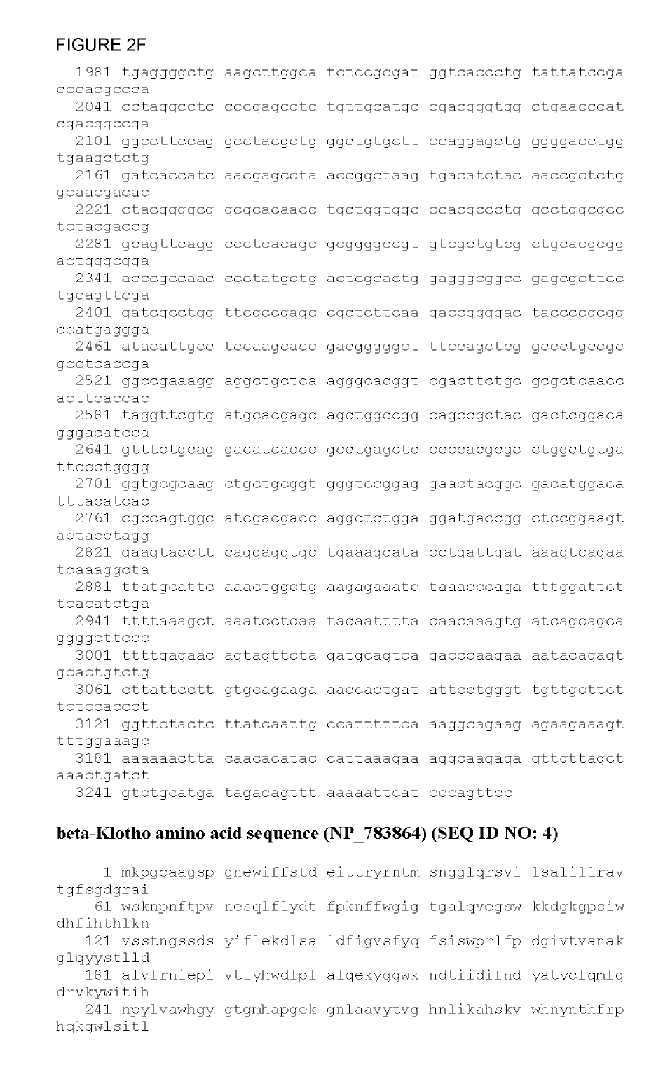

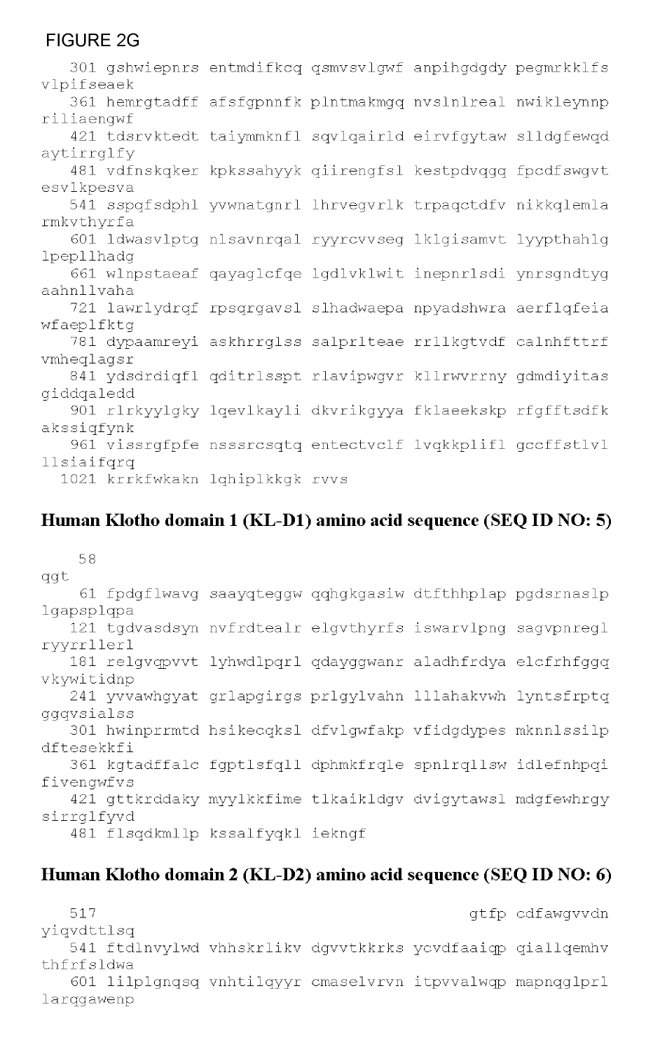

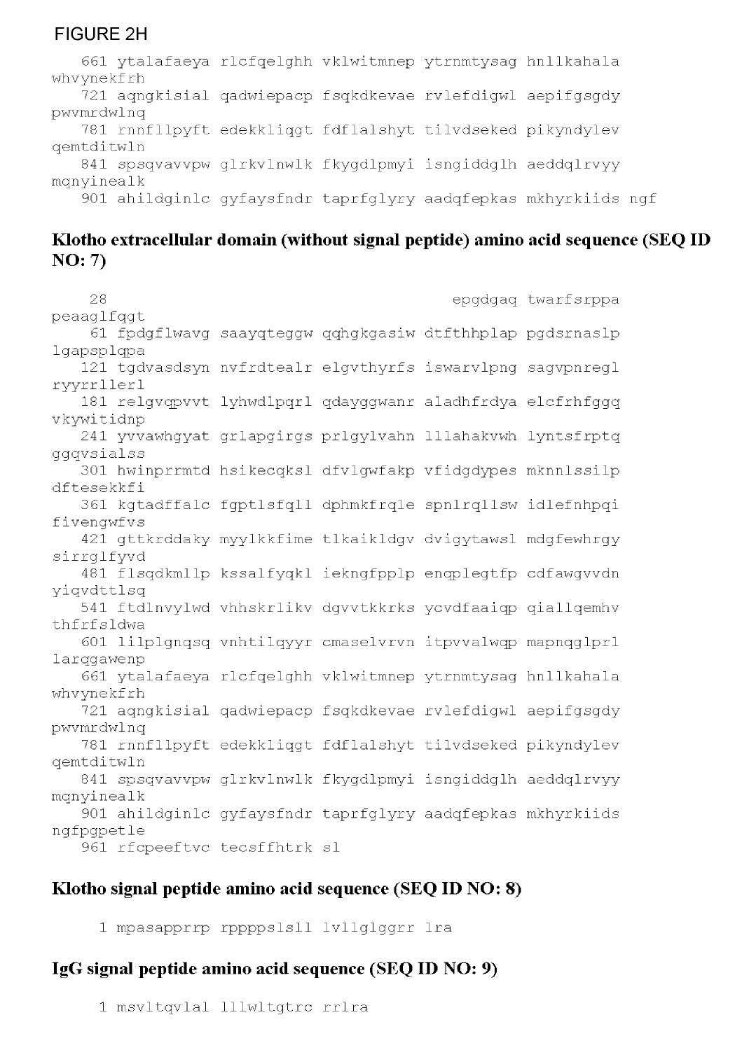

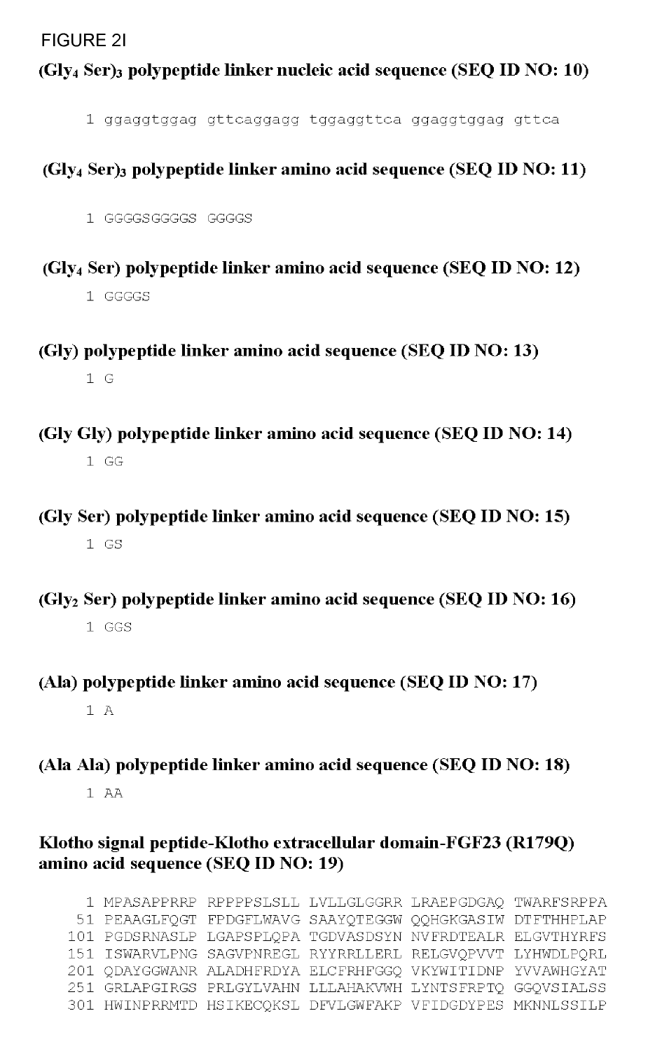

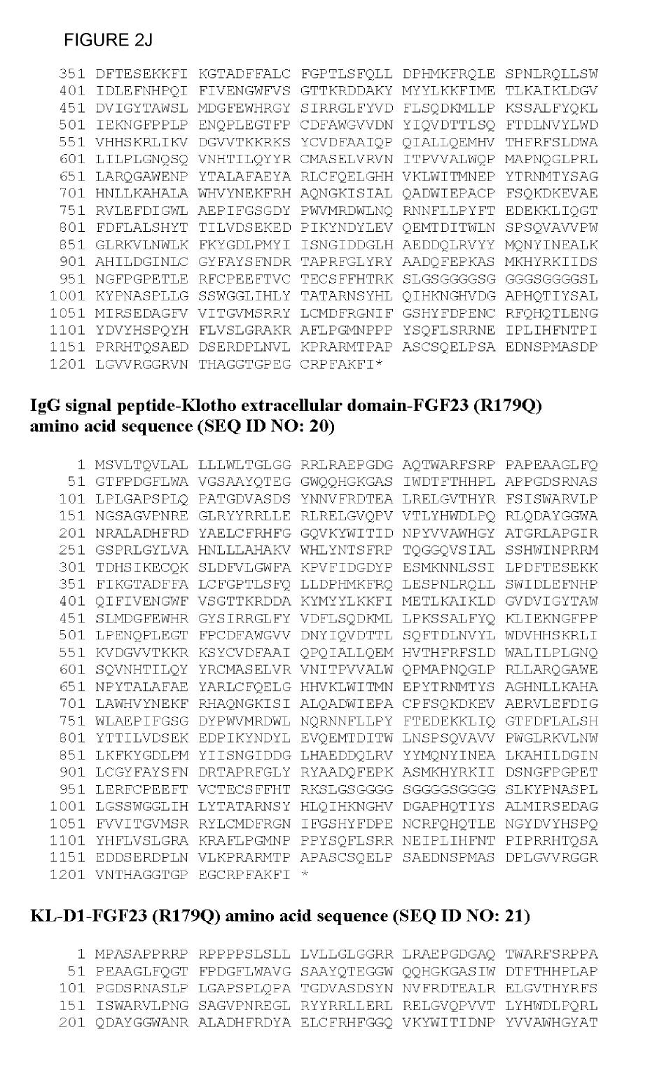

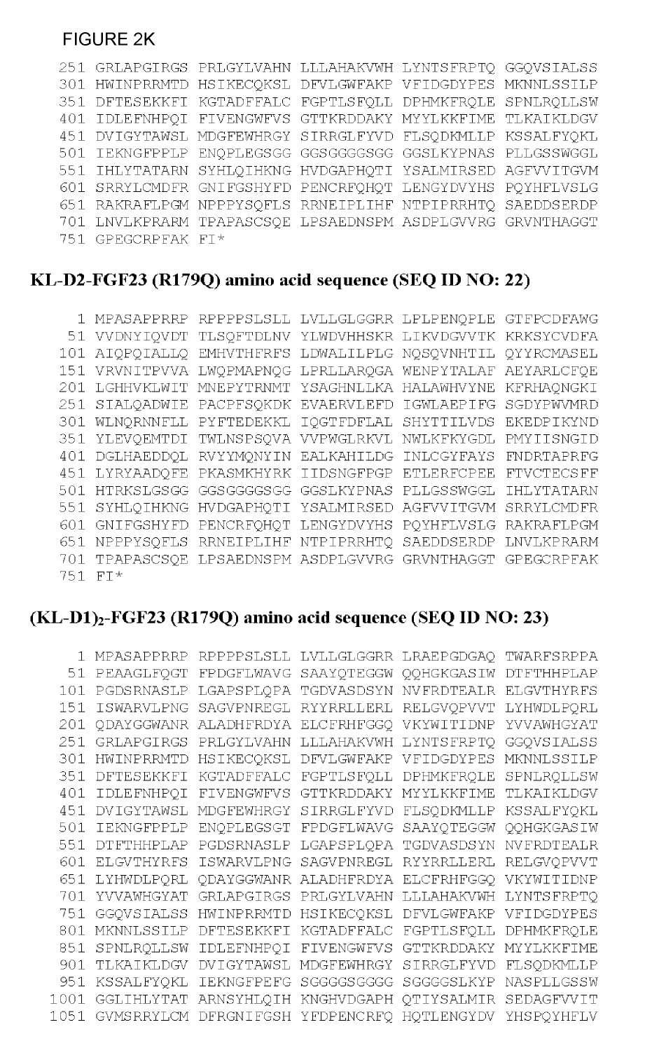

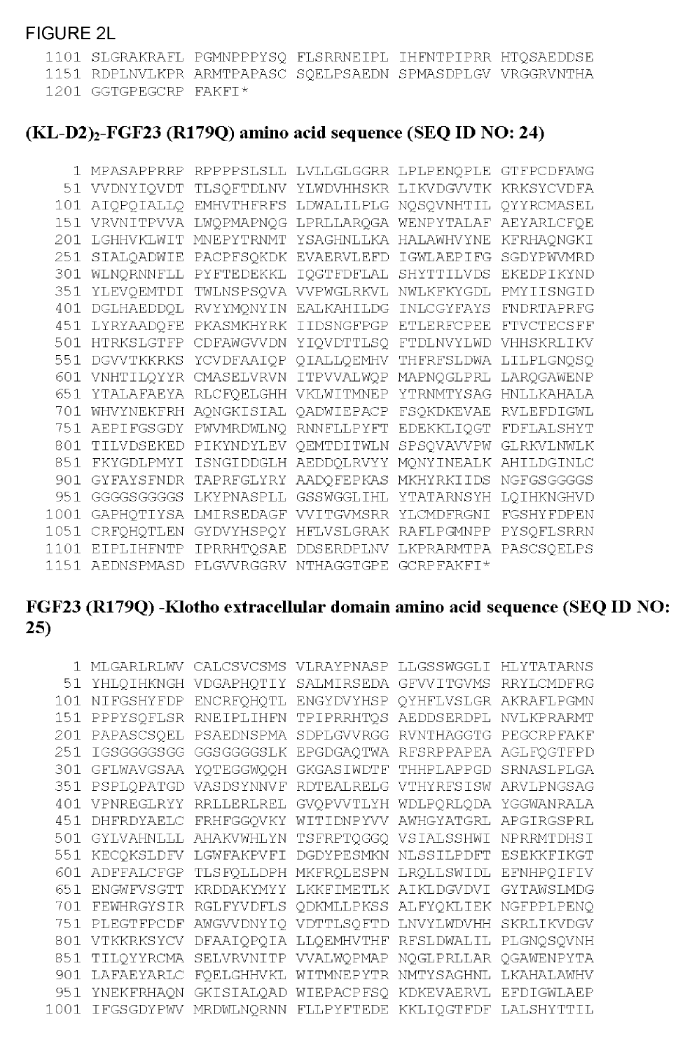

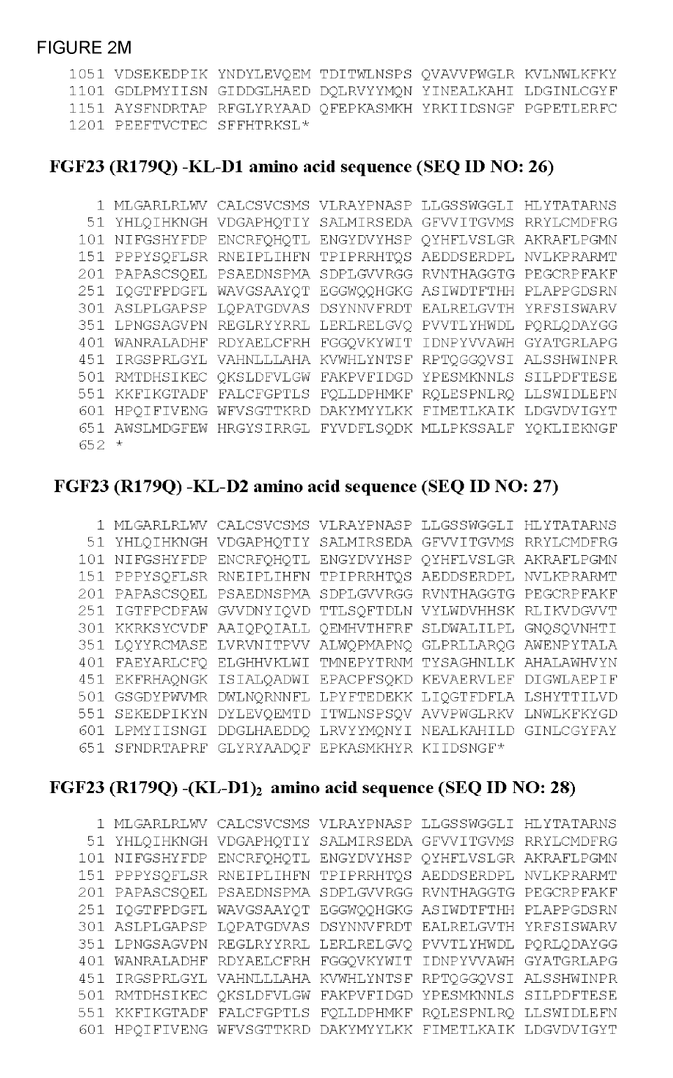

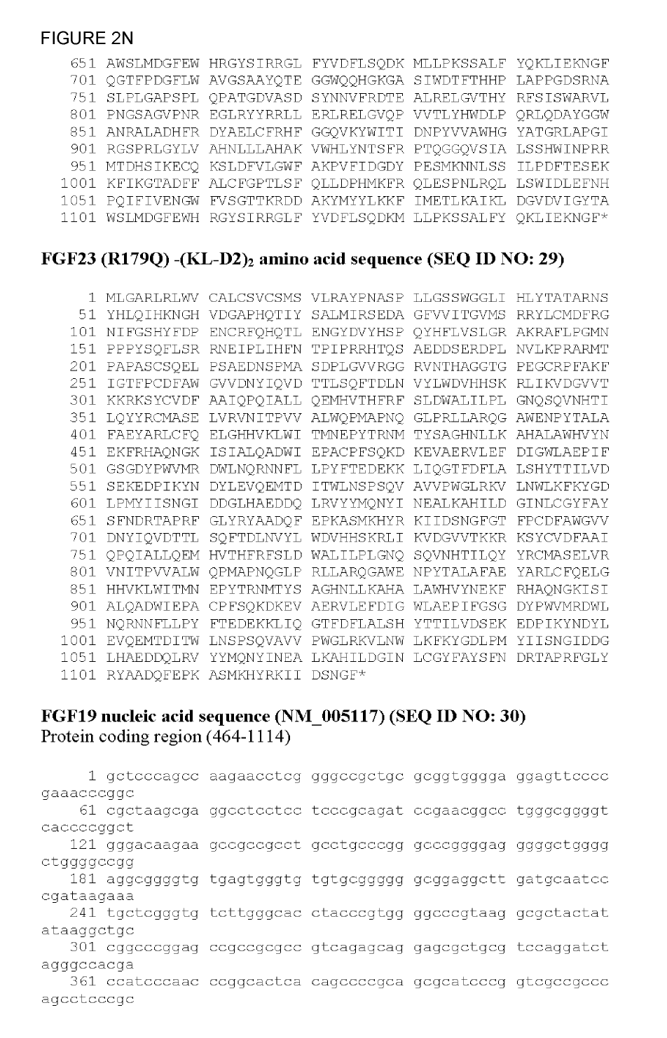

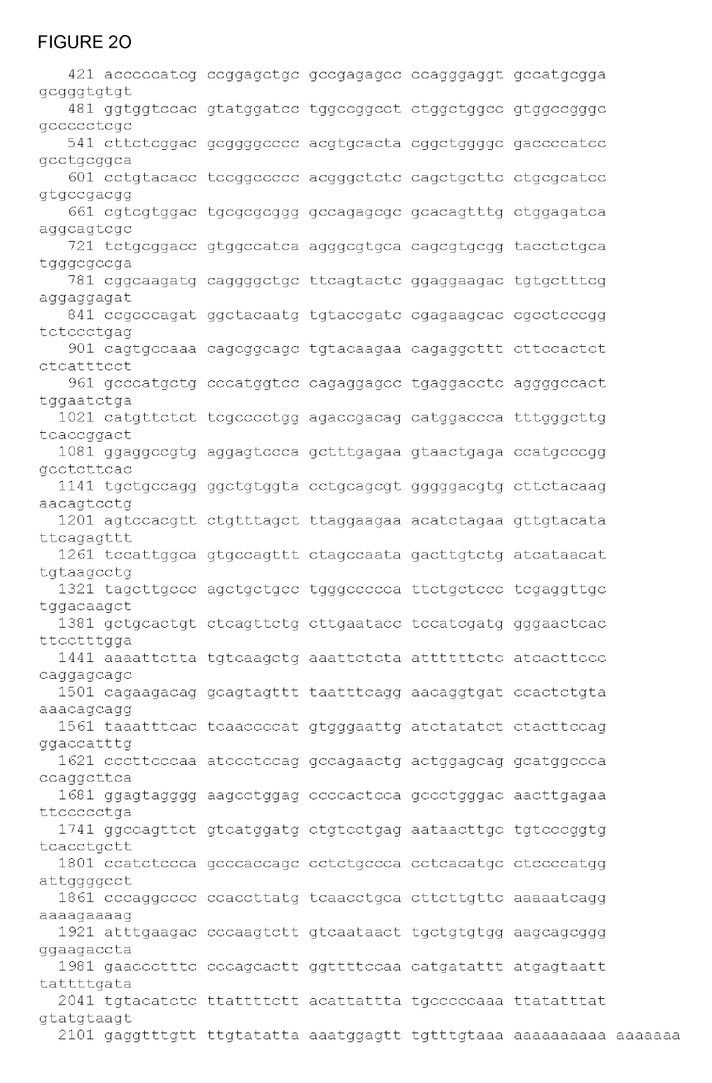

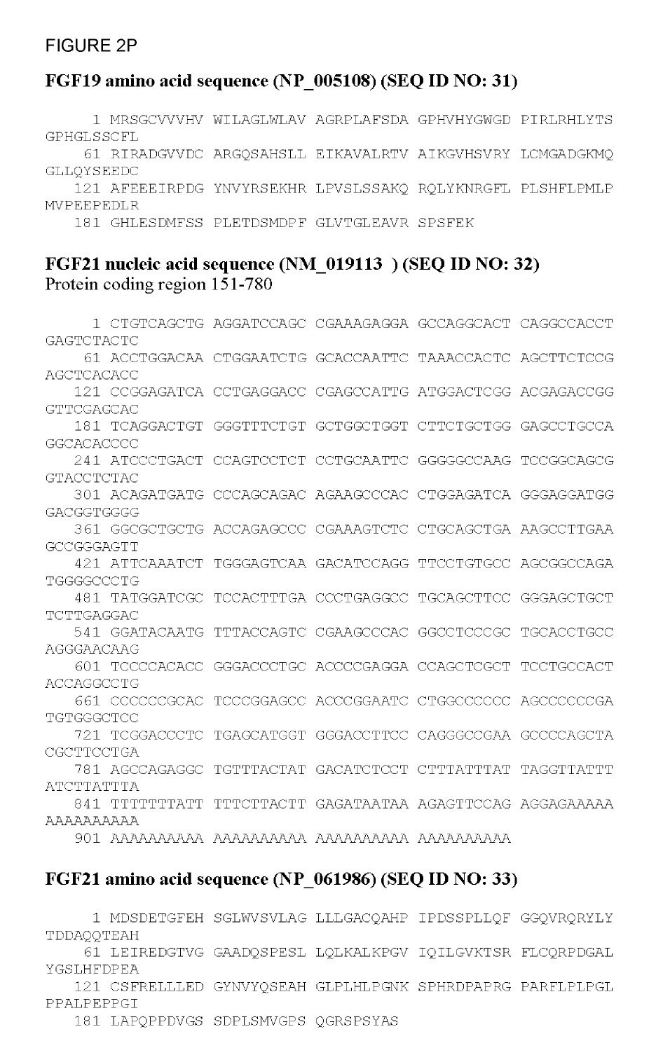

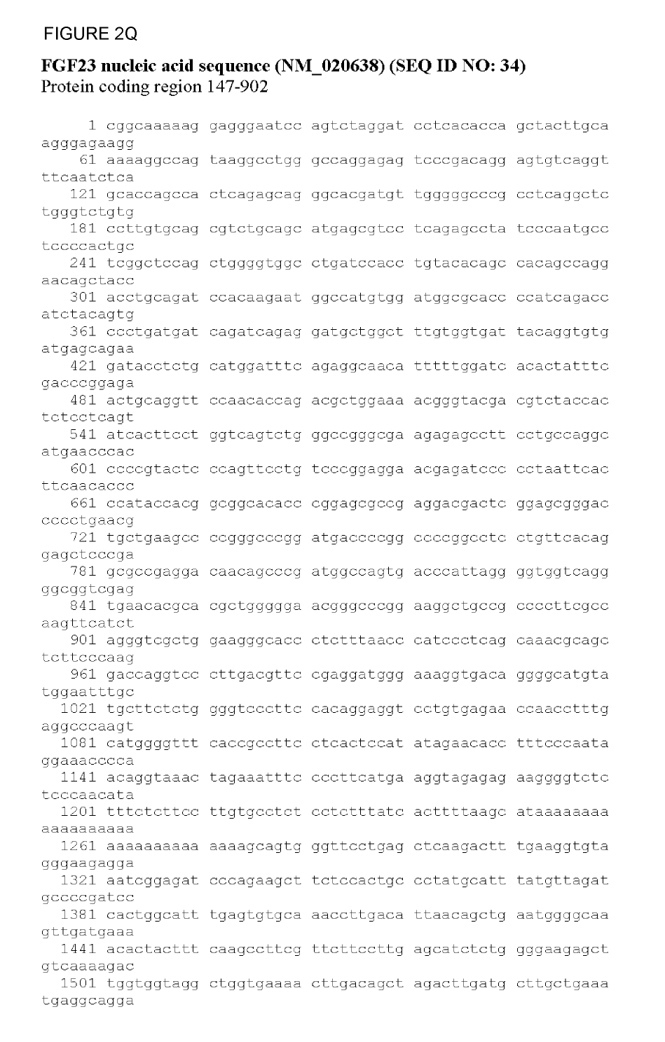

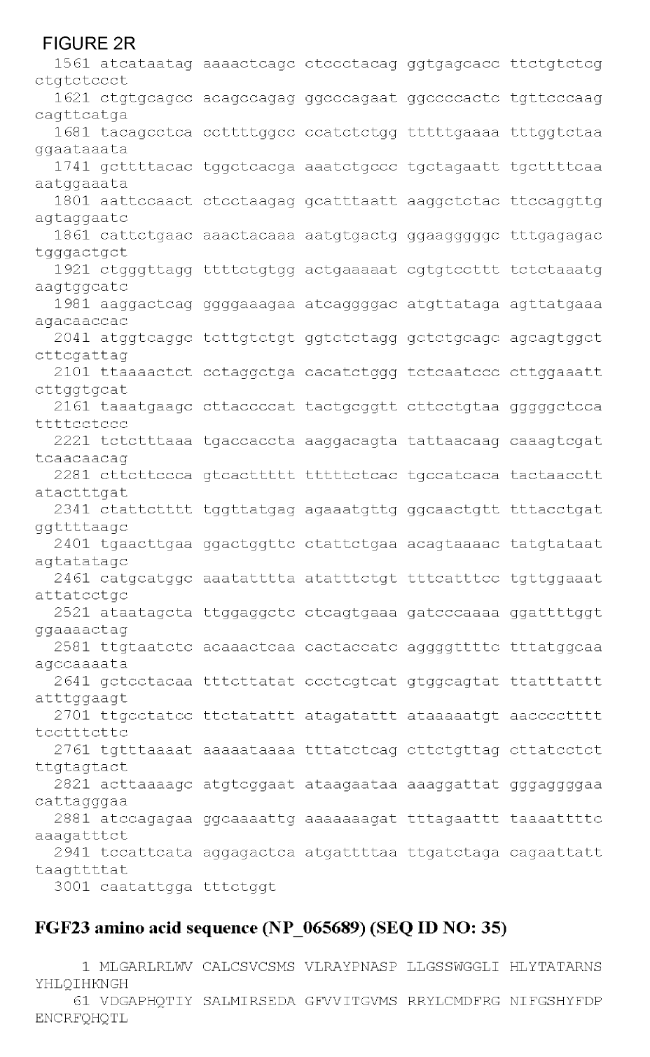

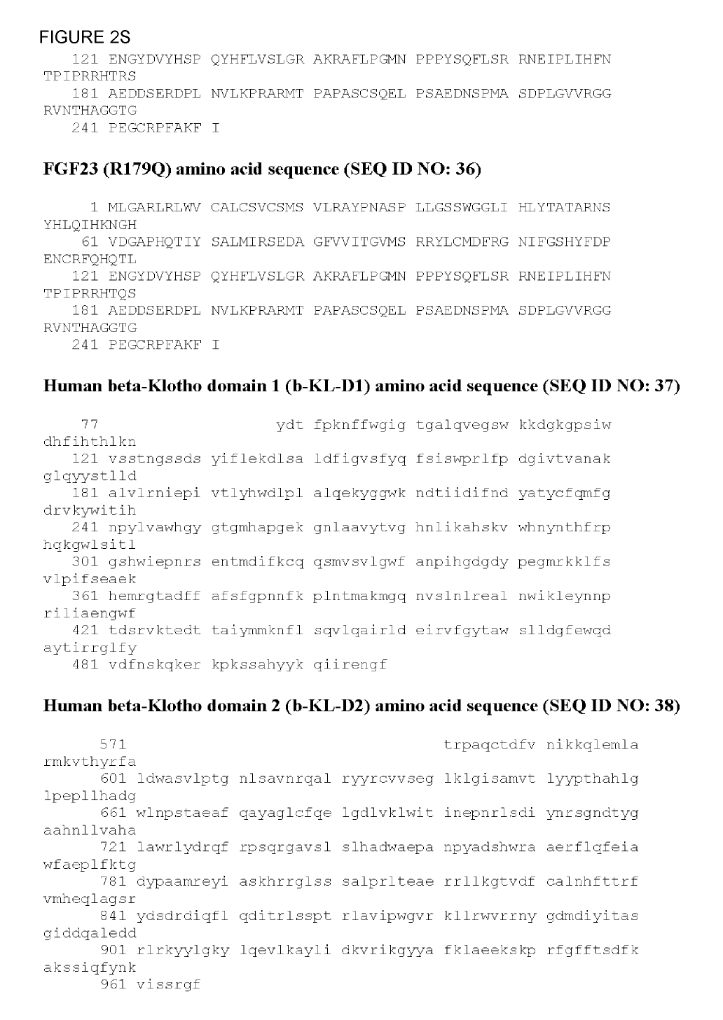

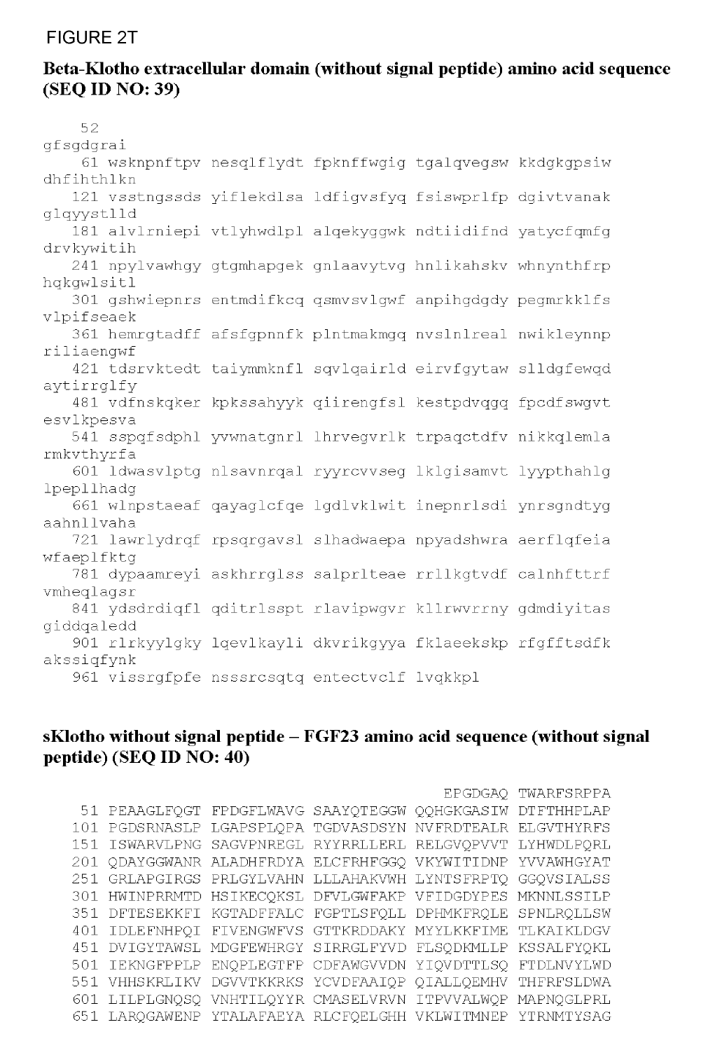

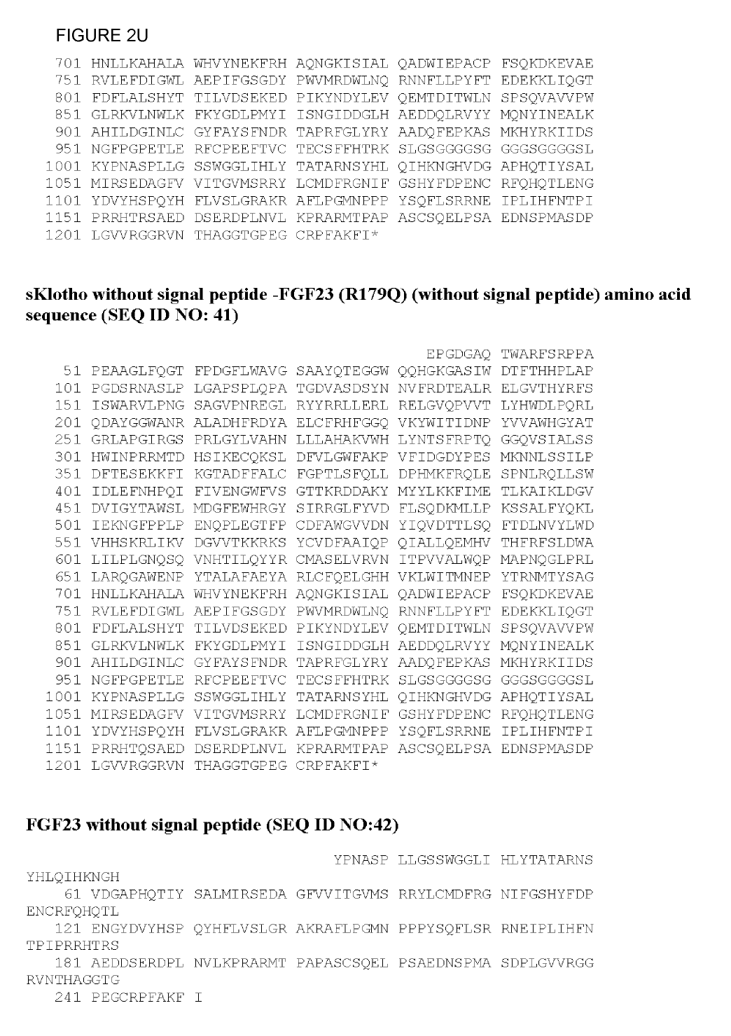

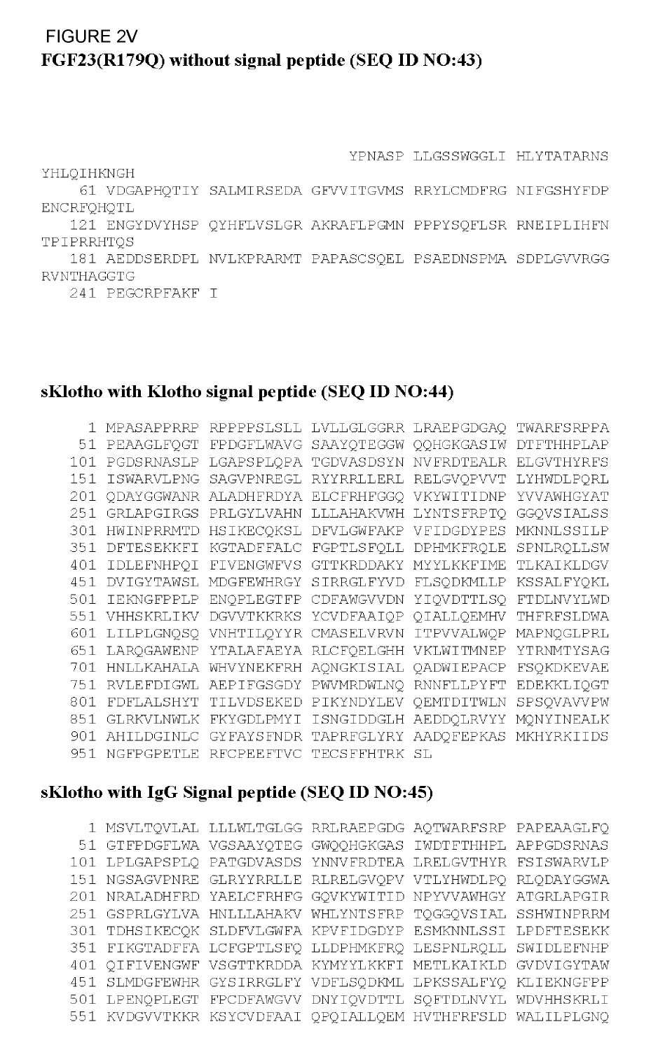

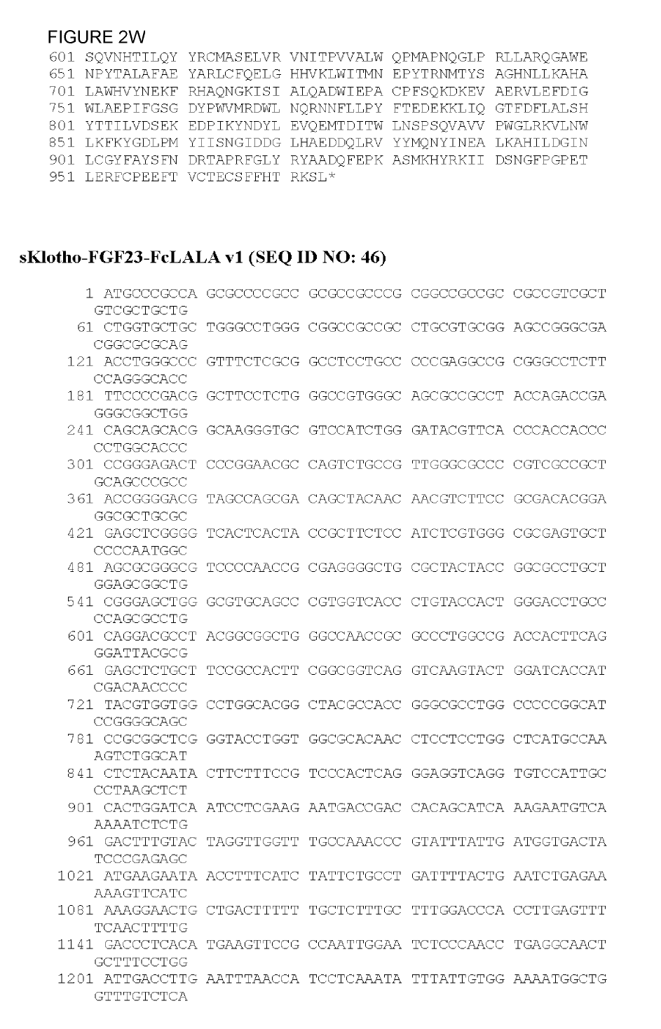

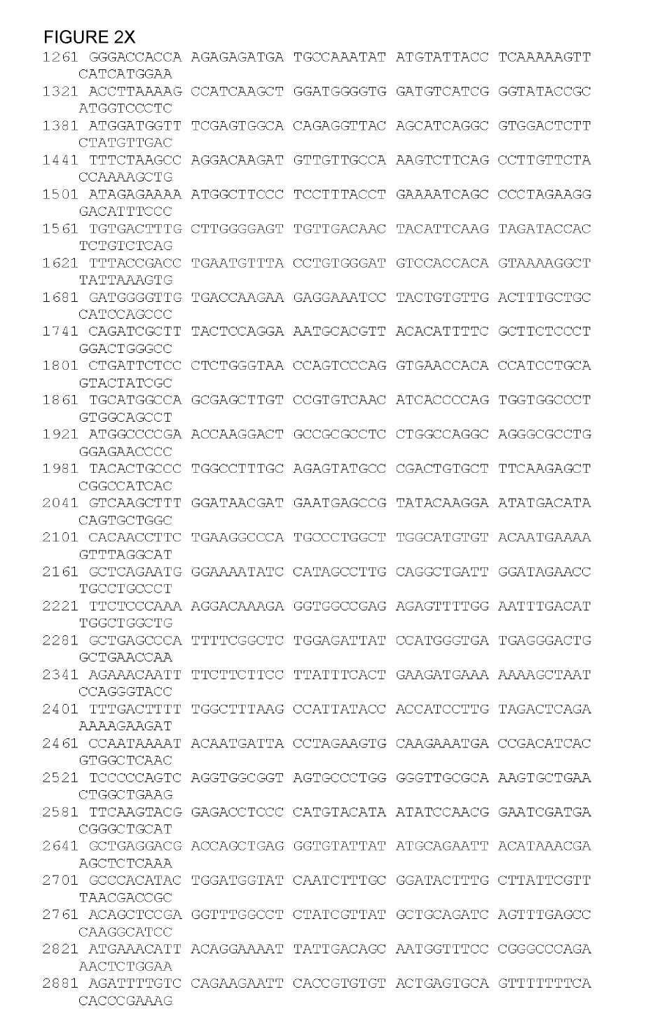

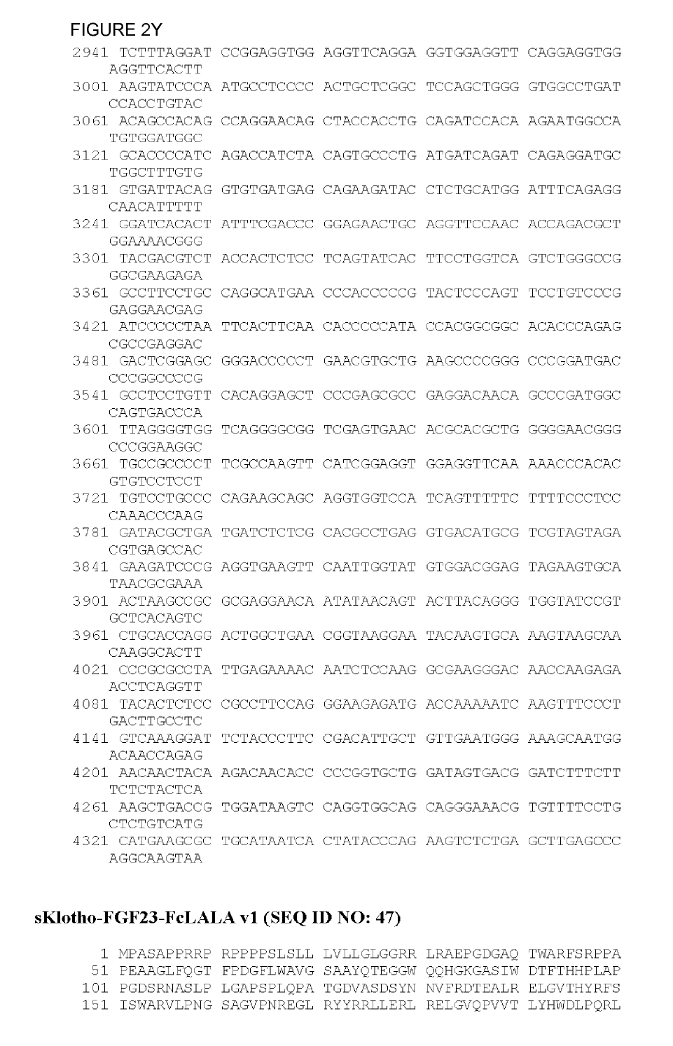

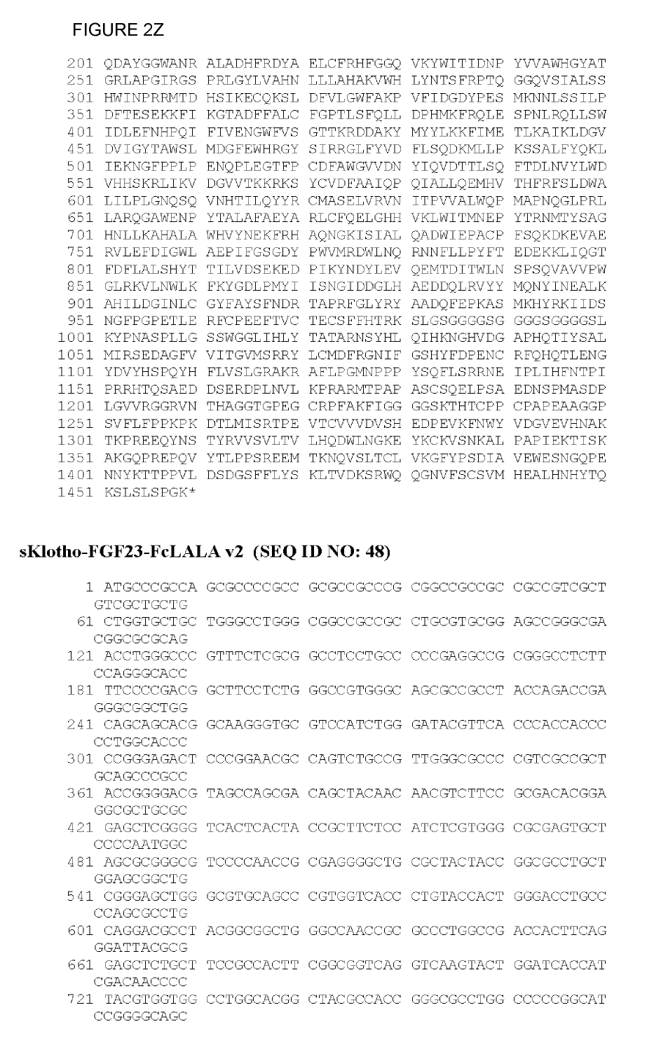

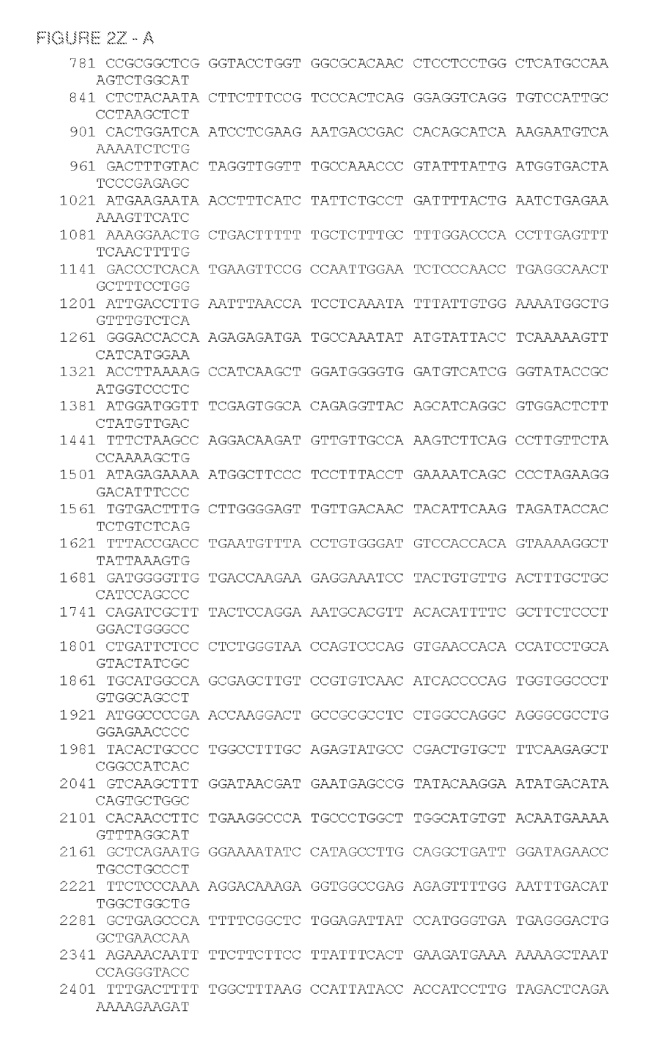

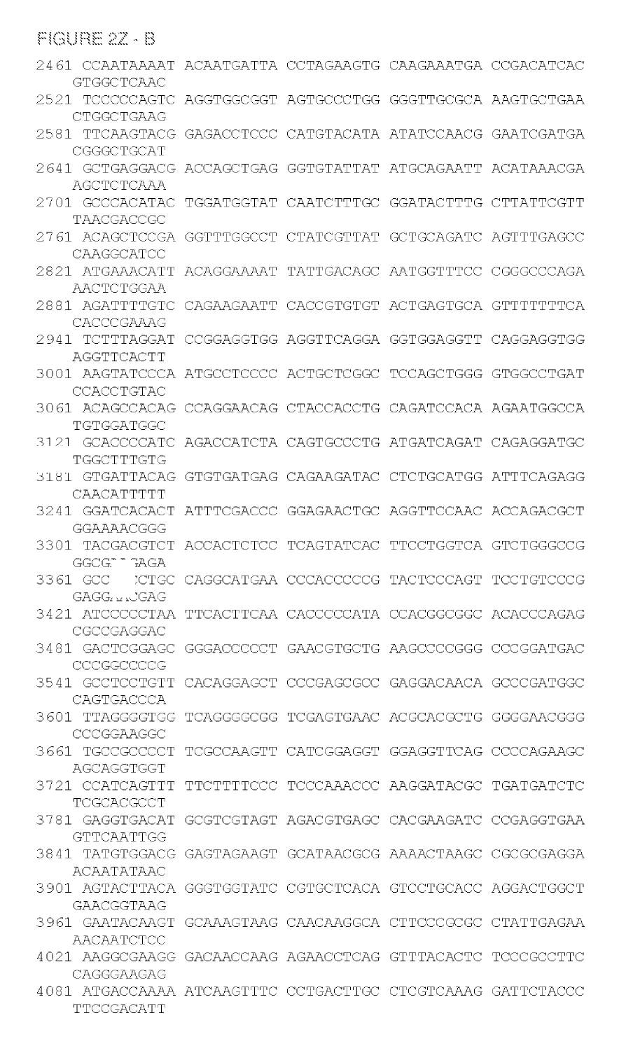

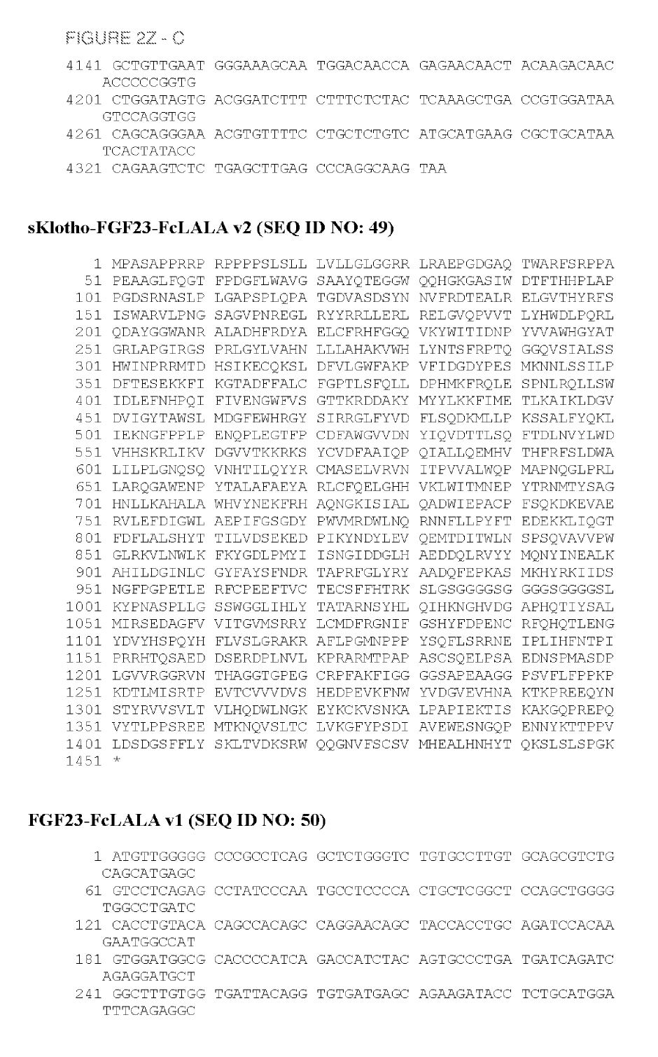

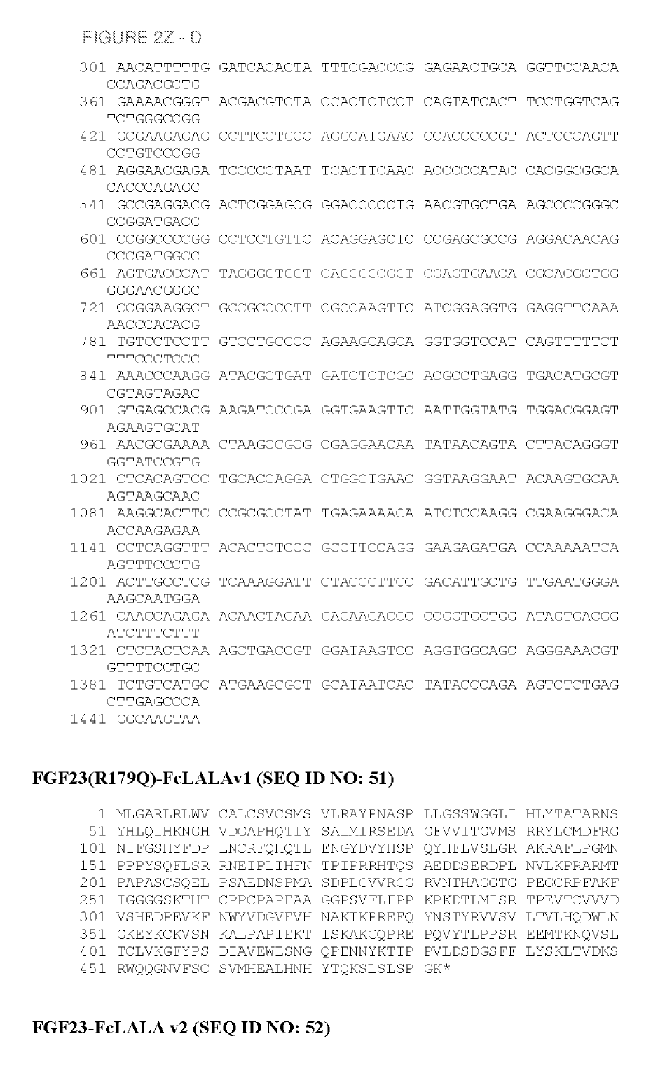

SEQUENCE LISTING (FIG. 2)

Human Klotho nucleic acid sequence (NM_004795)(SEQ ID NO: 1)
Protein region coding: 9-3047

| | |
|---|---|
| 1 | cgcgcagcat gcccgccagc gccccgccgc gccgcccgcg gccgccgccg ccgtcgctgt |
| 61 | cgctgctgct ggtgctgctg ggcctgggcg gccgccgcct gcgtgcggag ccgggcgacg |
| 121 | gcgcgcagac ctgggcccgt ttctcgcggc ctcctgcccc cgaggccgcg ggcctcttcc |
| 181 | agggcacctt ccccgacggc ttcctctggg ccgtgggcag cgccgcctac cagaccgagg |
| 241 | gcggctggca gcagcacggc aagggtgcgt ccatctggga tacgttcacc caccaccccc |
| 301 | tggcaccccc gggagactcc cggaacgcca gtctgccgtt gggcgccccg tcgccgctgc |
| 361 | agcccgccac cggggacgta gccagcgaca gctacaacaa cgtcttccgc gacacggagg |
| 421 | cgctgcgcga gctcggggtc actcactacc gcttctccat ctcgtgggcg cgagtgctcc |
| 481 | ccaatggcag cgcgggcgtc cccaaccgcg aggggctgcg ctactaccgg cgcctgctgg |
| 541 | agcggctgcg ggagctgggc gtgcagcccg tggtcaccct gtaccactgg gacctgcccc |
| 601 | agcgcctgca ggacgcctac ggcggctggg ccaaccgcgc cctggccgac cacttcaggg |
| 661 | attacgcgga gctctgcttc cgccacttcg gcggtcaggt caagtactgg atcaccatcg |
| 721 | acaaccccta cgtggtggcc tggcacggct acgccaccgg gcgcctggcc cccggcatcc |
| 781 | ggggcagccc gcggctcggg tacctggtgg cgcacaacct cctcctggct catgccaaag |
| 841 | tctggcatct ctacaatact tctttccgtc ccactcaggg aggtcaggtg tccattgccc |
| 901 | taagctctca ctggatcaat cctcgaagaa tgaccgacca cagcatcaaa gaatgtcaaa |
| 961 | aatctctgga ctttgtacta ggttggtttg ccaaacccgt atttattgat ggtgactatc |
| 1021 | ccgagagcat gaagaataac ctttcatcta ttctgcctga ttttactgaa tctgagaaaa |
| 1081 | agttcatcaa aggaactgct gacttttttg ctctttgctt tggacccacc ttgagttttc |
| 1141 | aacttttgga ccctcacatg aagttccgcc aattggaatc tcccaacctg aggcaactgc |
| 1201 | tttcctggat tgaccttgaa tttaaccatc ctcaaatatt tattgtggaa aatggctggt |
| 1261 | ttgtctcagg gaccaccaag agagatgatg ccaaatatat gtattacctc aaaaagttca |
| 1321 | tcatggaaac cttaaaagcc atcaagctgg atggggtgga tgtcatcggg tataccgcat |
| 1381 | ggtccctcat ggatggtttc gagtggcaca gaggttacag catcaggcgt ggactcttct |
| 1441 | atgttgactt tctaagccag gacaagatgt tgttgccaaa gtcttcagcc ttgttctacc |
| 1501 | aaaagctgat agagaaaaat ggcttccctc ctttacctga aaatcagccc ctagaaggga |
| 1561 | catttccctg tgactttgct tggggagttg ttgacaacta cattcaagta gataccactc |
| 1621 | tgtctcagtt taccgacctg aatgtttacc tgtgggatgt ccaccacagt aaaaggctta |
| 1681 | ttaaagtgga tggggttgtg accaagaaga ggaaatccta ctgtgttgac tttgctgcca |
| 1741 | tccagcccca gatcgcttta ctccaggaaa tgcacgttac acattttcgc ttctccctgg |
| 1801 | actgggccct gattctccct ctgggtaacc agtcccaggt gaaccacacc atcctgcagt |
| 1861 | actatcgctg catggccagc gagcttgtcc gtgtcaacat caccccagtg gtggccctgt |
| 1921 | ggcagcctat ggccccgaac caaggactgc cgcgcctcct ggccaggcag ggcgcctggg |
| 1981 | agaaccccta cactgccctg gcctttgcag agtatgcccg actgtgcttt caagagctcg |
| 2041 | gccatcacgt caagctttgg ataacgatga atgagccgta tacaaggaat atgacataca |
| 2101 | gtgctggcca caaccttctg aaggcccatg ccctggcttg gcatgtgtac aatgaaaagt |
| 2161 | ttaggcatgc tcagaatggg aaaatatcca tagccttgca ggctgattgg atagaacctg |
| 2221 | cctgcccttt ctcccaaaag gacaaagagg tggccgagag agttttggaa tttgacattg |
| 2281 | gctggctggc tgagcccatt ttcggctctg gagattatcc atgggtgatg agggactggc |
| 2341 | tgaaccaaag aaacaatttt cttcttcctt atttcactga agatgaaaaa aagctaatcc |
| 2401 | agggtacctt tgactttttg gctttaagcc attataccac catccttgta gactcagaaa |
| 2461 | aagaagatcc aataaaatac aatgattacc tagaagtgca agaaatgacc gacatcacgt |
| 2521 | ggctcaactc ccccagtcag gtggcggtag tgccctgggg gttgcgcaaa gtgctgaact |
| 2581 | ggctgaagtt caagtacgga gacctcccca tgtacataat atccaacgga atcgatgacg |
| 2641 | ggctgcatgc tgaggacgac cagctgaggg tgtattatat gcagaattac ataaacgaag |
| 2701 | ctctcaaagc ccacatactg gatggtatca atctttgcgg atactttgct tattcgttta |
| 2761 | acgaccgcac agctccgagg tttggcctct atcgttatgc tgcagatcag tttgagccca |
| 2821 | aggcatccat gaaacattac aggaaaatta ttgacagcaa tggtttcccg ggcccagaaa |
| 2881 | ctctggaaag attttgtcca gaagaattca ccgtgtgtac tgagtgcagt ttttttcaca |
| 2941 | cccgaaagtc tttactggct ttcatagctt ttctattttt tgcttctatt atttctctct |
| 3001 | cccttatatt ttactactcg aagaaaggca gaagaagtta caaatagttc tgaacatttt |
| 3061 | tctattcatt cattttgaaa taattatgca gacacatcag ctgttaacca tttgcacctc |
| 3121 | taagtgttgt gaaactgtaa atttcataca tttgacttct agaaaacatt tttgtggctt |
| 3181 | atgacagagg ttttgaaatg ggcataggtg atcgtaaaat attgaataat gcgaatagtg |
| 3241 | cctgaatttg ttctcttttt gggtgattaa aaaactgaca ggcactataa tttctgtaac |
| 3301 | acactaacaa aagcatgaaa aataggaacc acaccaatgc aacatttgtg cagaaatttg |
| 3361 | aatgacaaga ttaggaatat tttcttctgc acccacttct aaatttaatg tttttctgga |
| 3421 | agtagtaatt gcaagagttc gaatagaaag ttatgtacca agtaaccatt tctcagctgc |
| 3481 | cataataatg cctagtggct tcccctctgt caaatctagt ttcctatgga aaagaagatg |
| 3541 | gcagatacag gagagacgac agagggtcct aggctggaat gttcctttcg aaagcaatgc |
| 3601 | ttctatcaaa tactagtatt aatttatgta tctggttaat gacatacttg gagagcaaat |
| 3661 | tatggaaatg tgtattttat atgatttttg aggtcctgtc taaaccctgt gtccctgagg |
| 3721 | gatctgtctc actggcatct tgttgagggc cttgcacata ggaaactttt gataagtatc |
| 3781 | tgcggaaaaa caaacatgaa tcctgtgata ttgggctctt caggaagcat aaagcaattg |
| 3841 | tgaaatacag tataccgcag tggctctagg tggaggaaag gaggaaaaag tgcttattat |
| 3901 | gtgcaacatt atgattaatc tgattataca ccatttttga gcagatcttg gaatgaatga |
| 3961 | catgaccttt ccctagagaa taaggatgaa ataatcactc attctatgaa cagtgacact |
| 4021 | actttctatt ctttagctgt actgtaattt ctttgagttg atagttttac aaattcttaa |
| 4081 | taggttcaaa agcaatctgg tctgaataac actggatttg tttctgtgat ctctgaggtc |
| 4141 | tattttatgt ttttgctgct acttctgtgg aagtagcttt gaactagttt tactttgaac |
| 4201 | tttcacgctg aaacatgcta gtgatatcta gaaagggcta attaggtctc atcctttaat |
| 4261 | gccccttaaa taagtcttgc tgattttcag acagggaagt ctctctatta cactggagct |
| 4321 | gttttataga taagtcaata ttgtatcagg caagataaac caatgtcata acaggcattg |
| 4381 | ccaacctcac tgacacaggg tcatagtgta taataatata ctgtactata taatatatca |
| 4441 | tctttagagg tatgattttt tcatgaaaga taagcttttg gtaatattca ttttaaagtg |
| 4501 | gacttattaa aattggatgc tagagaatca agtttatttt atgtatatat ttttctgatt |
| 4561 | ataagagtaa tatatgttca ttgtaaaaat ttttaaaaca cagaaactat atgcaaagaa |
| 4621 | aaaataaaaa ttatctataa tctcagaacc cagaaatagc cactattaac atttcctacg |
| 4681 | tattttattt tacatagatc atattgtata tagttagtat ctttattaat ttttattatg |
| 4741 | aaactttcct ttgtcattat tagtcttcaa aagcatgatt tttaatagtt gttgagtatt |
| 4801 | ccaccacagg aatgtatcac aacttaaccg ttcccgtttg ttagactagt ttcttattaa |
| 4861 | tgttgatgaa tgttgtttaa aaataatttt gttgctacat ttactttaat ttccttgact |
| 4921 | gtaaagagaa gtaattttgc tccttgataa agtattatat taataataaa tctgcctgca |
| 4981 | actttttgcc ttctttcata ate |

Klotho amino acid sequence (NP_004786) (SEQ ID NO: 2)

| | |
|---|---|
| 1 | MPASAPPRRPRP PPPSLSLL LVLLGLGGRR LRAEPGDGAQ TWARFSRPPA PEAAGLFQGT |
| 61 | FPDGFLWAVG SAAYQTEGGW QQHGKGASIW DTFTHHPLAP PGDSRNASLP LGAPSPLQPA |
| 121 | TGDVASDSYN NVFRDTEALR ELGVTHYRFS ISWARVLPNG SAGVPNREGL RYYRRLLERL |
| 181 | RELGVQPVVT LYHWDLPQRL QDAYGGWANR ALADHFRDYA ELCFRHFGGQ VKYWITIDNP |
| 241 | YVVAWHGYAT GRLAPGIRGS PRLGYLVAHN LLLAHAKVWH LYNTSFRPTQ GGQVSIALSS |
| 301 | HWINPRRMTD HSIKECQKSL DFVLGWFAKP VFIDGDYPES MKNNLSSILP DFTESEKKFI |
| 361 | KGTADFFALC FGPTLSFQLL DPHMKFRQLE SPNLRQLLSW IDLEFNHPQI FIVENGWFVS |
| 421 | GTTKRDDAKY MYYLKKFIME TLKAIKLDGV DVIGYTAWSL MDGFEWHRGY SIRRGLFYVD |
| 481 | FLSQDKMLLP KSSALFYQKL IEKNGFPPLP ENQPLEGTFP CDFAWGWDN YIQVDTTLSQ |
| 541 | FTDLNVYLWD VHHSKRLIKV DGVVTKKRKS YCVDFAAIQP QIALLQEMHV THFRFSLDWA |
| 601 | LILPLGNQSQ VNHTILQYYR CMASELVRVN ITPVVALWQP MAPNQGLPRL LARQGAWENP |
| 661 | YTALAFAEYA RLCFQELGHH VKLWITMNEP YTRNMTYSAG HNLLKAHALA WHVYNEKFRH |
| 721 | AQNGKISIAL QADWIEPACP FSQKDKEVAE RVLEFDIGWL AEPIFGSGDY PWVMRDWLNQ |
| 781 | RNNFLLPYFT EDEKKLIQGT FDFLALSHYT TILVDSEKED PIKYNDYLEV QEMTDITWLN |
| 841 | SPSQVAVVPW GLRKVLNWLK FKYGDLPMYI ISNGIDDGLH AEDDQLRVYY MQNYINEALK |
| 901 | AHILDGINLC GYFAYSFNDR TAPRFGLYRY AADQFEPKAS MKHYRKIIDS NGFPGPETLE |
| 961 | RFCPEEFTVC TECSFFHTRK SLLAFIAFLF FASIISLSLI FYYSKKGRRS YK |

beta-Klotho nucleic acid sequence (NM_175737) (SEQ ID NO: 3)
Protein coding region: 98-3232

| | |
|---|---|
| 1 | atcctcagtc tcccagttca agctaatcat tgacagagct ttacaatcac aagcttttac |
| 61 | tgaagctttg ataagacagt ccagcagttg gtggcaaatg aagccaggct gtgcggcagg |
| 121 | atctccaggg aatgaatgga ttttcttcag cactgatgaa ataaccacac gctataggaa |
| 181 | tacaatgtcc aacgggggat tgcaaagatc tgtcatcctg tcagcactta ttctgctacg |
| 241 | agctgttact ggattctctg gagatggaag agctatatgg tctaaaaatc ctaattttac |
| 301 | tccggtaaat gaaagtcagc tgtttctcta tgacactttc cctaaaaact ttttctgggg |
| 361 | tattgggact ggagcattgc aagtggaagg gagttggaag aaggatggaa aaggaccttc |
| 421 | tatatgggat catttcatcc acacacacct taaaaatgtc agcagcacga atggttccag |
| 481 | tgacagttat atttttctgg aaaaagactt atcagccctg gattttatag gagtttcttt |
| 541 | ttatcaattt tcaatttcct ggccaaggct tttccccgat ggaatagtaa cagttgccaa |
| 601 | cgcaaaaggt ctgcagtact acagtactct tctggacgct ctagtgctta gaaacattga |
| 661 | acctatagtt actttatacc actgggattt gcctttggca ctacaagaaa aatatggggg |
| 721 | gtggaaaaat gataccataa tagatatctt caatgactat gccacatact gtttccagat |
| 781 | gtttggggac cgtgtcaaat attggattac aattcacaac ccatatctag tggcttggca |
| 841 | tgggtatggg acaggtatgc atgcccctgg agagaaggga aatttagcag ctgtctacac |
| 901 | tgtgggacac aacttgatca aggctcactc gaaagtttgg cataactaca acacacattt |
| 961 | ccgcccacat cagaagggtt ggttatcgat cacgttggga tctcattgga tcgagccaaa |
| 1021 | ccggtcggaa aacacgatgg atatattcaa atgtcaacaa tccatggttt ctgtgcttgg |
| 1081 | atggtttgcc aaccctatcc atggggatgg cgactatcca gaggggatga gaaagaagtt |
| 1141 | gttctccgtt ctacccattt tctctgaagc agagaagcat gagatgagag gcacagctga |
| 1201 | tttctttgcc ttttcttttg gacccaacaa cttcaagccc ctaaacacca tggctaaaat |
| 1261 | gggacaaaat gtttcactta atttaagaga agcgctgaac tggattaaac tggaatacaa |
| 1321 | caaccctcga atcttgattg ctgagaatgg ctggttcaca gacagtcgtg tgaaaacaga |
| 1381 | agacaccacg gccatctaca tgatgaagaa tttcctcagc caggtgcttc aagcaataag |
| 1441 | gttagatgaa atacgagtgt ttggttatac tgcctggtct ctcctggatg gctttgaatg |
| 1501 | gcaggatgct tacaccatcc gccgaggatt attttatgtg gattttaaca gtaaacagaa |
| 1561 | agagcggaaa cctaagtctt cagcacacta ctacaaacag atcatacgag aaaatggttt |
| 1621 | ttctttaaaa gagtccacgc cagatgtgca gggccagttt ccctgtgact tctcctgggg |
| 1681 | tgtcactgaa tctgttctta agcccgagtc tgtggcttcg tccccacagt tcagcgatcc |
| 1741 | tcatctgtac gtgtggaacg ccactggcaa cagactgttg caccgagtgg aaggggtgag |
| 1801 | gctgaaaaca cgacccgctc aatgcacaga ttttgtaaac atcaaaaaac aacttgagat |
| 1861 | gttggcaaga atgaaagtca cccactaccg gtttgctctg gattgggcct cggtccttcc |
| 1921 | cactggcaac ctgtccgcgg tgaaccgaca ggccctgagg tactacaggt gcgtggtcag |
| 1981 | tgaggggctg aagcttggca tctccgcgat ggtcaccctg tattatccga cccacgccca |
| 2041 | cctaggcctc cccgagcctc tgttgcatgc cgacgggtgg ctgaacccat cgacggccga |
| 2101 | ggccttccag gcctacgctg ggctgtgctt ccaggagctg ggggacctgg tgaagctctg |
| 2161 | gatcaccatc aacgagccta accggctaag tgacatctac aaccgctctg gcaacgacac |
| 2221 | ctacggggcg gcgcacaacc tgctggtggc ccacgccctg gcctggcgcc tctacgaccg |
| 2281 | gcagttcagg ccctcacagc gcggggccgt gtcgctgtcg ctgcacgcgg actgggcgga |
| 2341 | acccgccaac ccctatgctg actcgcactg gagggcggcc gagcgcttcc tgcagttcga |
| 2401 | gatcgcctgg ttcgccgagc cgctcttcaa gaccggggac taccccgcgg ccatgaggga |
| 2461 | atacattgcc tccaagcacc gacgggggct ttccagctcg gccctgccgc gcctcaccga |
| 2521 | ggccgaaagg aggctgctca agggcacggt cgacttctgc gcgctcaacc acttcaccac |
| 2581 | taggttcgtg atgcacgagc agctggccgg cagccgctac gactcggaca gggacatcca |
| 2641 | gtttctgcag gacatcaccc gcctgagctc ccccacgcgc ctggctgtga ttccctgggg |
| 2701 | ggtgcgcaag ctgctgcggt gggtccggag gaactacggc gacatggaca tttacatcac |
| 2761 | cgccagtggc atcgacgacc aggctctgga ggatgaccgg ctccggaagt actacctagg |
| 2821 | gaagtacctt caggaggtgc tgaaagcata cctgattgat aaagtcagaa tcaaaggcta |
| 2881 | ttatgcattc aaactggctg aagagaaatc taaacccaga tttggattct tcacatctga |
| 2941 | ttttaaagct aaatcctcaa tacaatttta caacaaagtg atcagcagca ggggcttccc |
| 3001 | ttttgagaac agtagttcta gatgcagtca gacccaagaa aatacagagt gcactgtctg |
| 3061 | cttattcctt gtgcagaaga aaccactgat attcctgggt tgttgcttct tctccaccct |
| 3121 | ggttctactc ttatcaattg ccatttttca aaggcagaag agaagaaagt tttggaaagc |
| 3181 | aaaaaactta caacacatac cattaaagaa aggcaagaga gttgttagct aaactgatct |
| 3241 | gtctgcatga tagacagttt aaaaattcat cccagttcc |

beta-Klotho amino acid sequence (NP_783864) (SEQ ID NO: 4)

| | |
|---|---|
| 1 | mkpgcaagsp gnewiffstd eittryrntm sngglqrsvi lsalillrav tgfsgdgral |
| 61 | wsknpnftpv nesqlflydt fpknffwgig tgalqvegsw kkdgkgpsiw dhfihthlkn |
| 121 | vsstngssds yiflekdlsa ldfigvsfyg fsiswprlfp dgivtvanak glqyystlld |
| 181 | alvlrniepi vtlyhwdlpl alqekyggwk ndtildifnd yatycfqmfg drvkywitih |
| 241 | npylvawhgy gtgmhapgek gnlaavytvg hnlikahskv whnynthfrp hqkgwlsitl |
| 301 | gshwiepnrs entmdifkcg gsmvsvlgwf anpihgdgdy pegmrkklfs vlpifseaek |
| 361 | hemrgtadff afsfgpnnfk pintmakmgq nvslnlreal nwikleynnp rillaengwf |
| 421 | tdsrvktedt talymmknfl sqvlgairld eirvfgytaw slldgfewqd aytirrglfy |
| 481 | vdfnskqker kpkssahyyk glirengfsl kestpdvggq fpcdfswgvt esvlkpesva |
| 541 | sspqfsdphl yvwnatgnrl lhrvegvrlk trpagctdfv nikkglemla rmkvthyrfa |
| 601 | ldwasvlptg nlsavnrgal ryyrcvvseg lklgisamvt lyypthahlg lpepllhadg |
| 661 | wlnpstaeaf gayaglcfge lgdlvklwit inepnrlsdi ynrsgndtyg aahnllvaha |
| 721 | lawrlydrqf rpsgrgaysl slhadwaepa npyadshwra aerflgfela wfaeplfktg |
| 781 | dypaamreyi askhrrglss salprlteae rrllkgtvdf calnhfttrf vmheqlagsr |
| 841 | ydsdrdigfl gditrlsspt rlavipwgvr kllrwvrrny gdmdlyitas giddqaledd |
| 901 | rlrkyylgky lgevlkayll dkvrikgyya fklaeekskp rfgfftsdfk akssiqfynk |
| 961 | vissrgfpfe nsssrcsgtg entectvclf lvgkkplifl gccffstivl llsialfgrq |
| 1021 | krrkfwkakn lghiplkkgk rvvs |

Human Klotho domain 1 (KL-D1) amino acid sequence (SEQ ID NO: 5)

| | |
|---|---|
| 58 | qgt |
| 61 | fpdgflwavg saayqteggw gghgkgasiw dtfthhplap pgdsrnaslp lgapsplqpa |
| 121 | tgdvasdsyn nvfrdtealr elgvthyrfs iswarvlpng sagvpnregl ryyrrllerl |
| 181 | relgvqpvvt lyhwdlpgrl qdayggwanr aladhfrdya elcfrhfggq vkywitidnp |
| 241 | yvvawhgyat grlapgirgs prlgylvahn lllahakvwh lyntsfrptq ggqvsialss |
| 301 | hwinprrmtd hsikecgksl dfvlgwfakp vfidgdypes mknnlssilp dftesekkfl |
| 361 | kgtadffalc fgptlsfqll dphmkfrqle spnlrqllsw idlefnhpgi fivengwfvs |
| 421 | gttkrddaky myylkkfime tlkalkldgv dvigytawsl mdgfewhrgy sirrglfyvd |
| 481 | flsqdkmllp kssalfyqkl lekngf |

Human Klotho domain 2 (KL-D2) amino acid sequence (SEQ ID NO: 6)

| | |
|---|---|
| 517 | gtfp cdfawgvvdn yiqvdttlsq |
| 541 | ftdlnvylwd vhhskrlikv dgvvtkkrks ycvdfaalgp giallgemhv thfrfsldwa |
| 601 | lllplgngsg vnhtilgyyr cmaselvrvn itpvvalwqp mapngglprl larqgawenp |
| 661 | ytalafaeya rlcfgelghh vklwitmnep ytrnmtysag hnllkahala whvynekfrh |
| 721 | agngkisial gadwiepacp fsqkdkevae rvlefdigwl aeplfgsgdy pwvmrdwlnq |
| 781 | rnnfllpyft edekkliggt fdflalshyt tilvdseked pikyndylev qemtditwln |
| 841 | spsqvavvpw glrkvinwlk fkygdlpmyi isngiddglh aeddqlrvyy mqnyinealk |
| 901 | ahildginlc gyfaysfndr taprfglyry aadqfepkas mkhyrkilds ngf |

Klotho extracellular domain (without signal peptide) amino acid sequence
(SEQ ID NO: 7)

| | |
|---|---|
| 28 | epgdgaq twarfsrppa peaaglfqgt |
| 61 | fpdgflwavg saayqteggw gghgkgasiw dtfthhplap pgdsrnaslp lgapsplqpa |
| 121 | tgdvasdsyn nvfrdtealr elgvthyrfs iswarvlpng sagvpnregl ryyrrllerl |
| 181 | relgvqpvvt lyhwdlpqrl qdayggwanr aladhfrdya elcfrhfggq vkywitidnp |
| 241 | yvvawhgyat grlapgirgs prlgylvahn lllahakvwh lyntsfrptq ggqvsialss |
| 301 | hwinprrmtd hsikecqksl dfvlgwfakp vfidgdypes mknnlssilp dftesekkfi |
| 361 | kgtadffalc fgptlsfqll dphmkfrqle spnlrqllsw idlefnhpqi fivengwfvs |
| 421 | gttkrddaky myylkkfime tlkaikldgv dvigytawsl mdgfewhrgy sirrglfyvd |
| 481 | flsqdkmllp kssalfyqkl iekngfpplp enqplegtfp cdfawgvvdn yiqvdttlsq |
| 541 | ftdlnvylwd vhhskrlikv dgvvtkkrks ycvdfaaiqp qiallqemhv thfrfsldwa |
| 601 | lilplgnqsq vnhtilqyyr cmaselvrvn itpvvalwqp mapnqglprl larqgawenp |
| 661 | ytalafaeya rlcfqelghh vklwitmnep ytrnmtysag hnllkahala whvynekfrh |
| 721 | aqngkisial qadwiepacp fsqkdkevae rvlefdigwl aepifgsgdy pwvmrdwlnq |
| 781 | rnnfllpyft edekkliqgt fdflalshyt tilvdseked pikyndylev qemtditwln |
| 841 | spsqvavvpw glrkvinwlk fkygdlpmyi isngiddglh aeddqlrvyy mqnyinealk |
| 901 | ahildginlc gyfaysfndr taprfglyry aadqfepkas mkhyrkiids ngfpgpetle |
| 961 | rfcpeeftvc tecsffhtrk sl |

Klotho signal peptide amino acid sequence (SEQ ID NO: 8)

| | |
|---|---|
| 1 | mpasapprrp rppppslsll lvllglggrr lra |

IgG signal peptide amino acid sequence (SEQ ID NO: 9)

| | |
|---|---|
| 1 | msvltqvlal lllwltgtrc rrlra |

(Gly₄ Ser)₃ polypeptide linker nucleic acid sequence (SEQ ID NO: 10)

| | |
|---|---|
| 1 | ggaggtggag gttcaggagg tggaggttca ggaggtggag gttca |

(Gly₄ Ser)₃ polypeptide linker amino acid sequence (SEQ ID NO: 11)

| | |
|---|---|
| 1 | GGGGSGGGGS GGGGS |

(Gly₄ Ser) polypeptide linker amino acid sequence (SEQ ID NO: 12)

| | |
|---|---|
| 1 | GGGGS |

(Gly) polypeptide linker amino acid sequence (SEQ ID NO: 13)

| | |
|---|---|
| 1 | G |

(Gly Gly) polypeptide linker amino acid sequence (SEQ ID NO: 14)

| | |
|---|---|
| 1 | GG |

(Gly Ser) polypeptide linker amino acid sequence (SEQ ID NO: 15)

| | |
|---|---|
| 1 | GS |

(G1y₂ Ser) polypeptide linker amino acid sequence (SEQ ID NO: 16)

| | |
|---|---|
| 1 | GGS |

(Ala) polypeptide linker amino acid sequence (SEQ ID NO: 17)

| | |
|---|---|
| 1 | A |

(Ala Ala) polypeptide linker amino acid sequence (SEQ ID NO: 18)

| | |
|---|---|
| 1 | AA |

Klotho signal peptide-Klotho extracellular domain-FGF23 (R179Q)
amino acid sequence (SEQ ID NO: 19)

| | |
|---|---|
| 1 | MPASAPPRRP RPPPPSLSLL LVLLGLGGRR LRAEPGDGAQ TWARFSRPPA |
| 51 | PEAAGLFQGT FPDGFLWAVG SAAYQTEGGW QQHGKGASIW DTFTHHPLAP |
| 101 | PGDSRNASLP LGAPSPLQPA TGDVASDSYN NVFRDTEALR ELGVTHYRFS |
| 151 | ISWARVLPNG SAGVPNREGL RYYRRLLERL RELGVQPVVT LYHWDLPQRL |
| 201 | QDAYGGWANR ALADHFRDYA ELCFRHFGGQ VKYWITIDNP YVVAWHGYAT |
| 251 | GRLAPGIRGS PRLGYLVAHN LLLAHAKVWH LYNTSFRPTQ GGQVSIALSS |
| 301 | HWINPRRMTD HSIKECQKSL DFVLGWFAKP VFIDGDYPES MKNNLSSILP |
| 351 | DFTESEKKFI KGTADFFALC FGPTLSFQLL DPHMKFRQLE SPNLRQLLSW |
| 401 | IDLEFNHPQI FIVENGWFVS GTTKRDDAKY MYYLKKFIME TLKAIKLDGV |
| 451 | DVIGYTAWSL MDGFEWHRGY SIRRGLFYVD FLSQDKMLLP KSSALFYQKL |
| 501 | IEKNGFPPLP ENQPLEGTFP CDFAWGVVDN YIQVDTTLSQ FTDLNVYLWD |
| 551 | VHHSKRLIKV DGVVTKKRKS YCVDFAAIQP QIALLQEMHV THFRFSLDWA |
| 601 | LILPLGNQSQ VNHTILQYYR CMASELVRVN ITPVVALWQP MAPNQGLPRL |
| 651 | LARQGAWENP YTALAFAEYA RLCFQELGHH VKLWITMNEP YTRNMTYSAG |
| 701 | HNLLKAHALA WHVYNEKFRH AQNGKISIAL QADWIEPACP FSQKDKEVAE |
| 751 | RVLEFDIGWL AEPIFGSGDY PWVMRDWLNQ RNNFLLPYFT EDEKKLIQGT |
| 801 | FDFLALSHYT TILVDSEKED PIKYNDYLEV QEMTDITWLN SPSQVATNPW |
| 851 | GLRKVLNWLK FKYGDLPMYI ISNGIDDGLH AEDDQLRVYY MQNYINEALK |
| 901 | AHILDGINLC GYFAYSFNDR TAPRFGLYRY AADQFEPKAS MKHYRKIIDS |
| 951 | NGFPGPETLE RFCPEEFTVC TECSFFHTRK SLGSGGGGSG GGGSGGGGSL |
| 1001 | KYPNASPLLG SSWGGLIHLY TATARNSYHL QIHKNGHVDG APHQTIYSAL |
| 1051 | MIRSEDAGFV VITGVMSRRY LCMDFRGNIF GSHYFDPENC RFQHQTLENG |
| 1101 | YDVYHSPQYH FLVSLGRAKR AFLPGMNPPP YSQFLSRRNE IPLIHFNTPI |
| 1151 | PRRHTQSAED DSERDPLNVL KPRARMTPAP ASCSQELPSA EDNSPMASDP |
| 1201 | LGWRGGRVN THAGGTGPEG CRPFAKFI* |

IgG signal peptide-Klotho extracellular domain-FGF23 (R179Q)
amino acid sequence (SEQ ID NO: 20)

| | |
|---|---|
| 1 | MSVLTQVLAL LLLWLTGLGG RRLRAEPGDG AQTWARFSRP PAPEAAGLFQ |
| 51 | GTFPDGFLWA VGSAAYQTEG GWQQHGKGAS IWDTFTHHPL APPGDSRNAS |
| 101 | LPLGAPSPLQ PATGDVASDS YNNVFRDTEA LRELGVTHYR FSISWARVLP |
| 151 | NGSAGVPNRE GLRYYRRLLE RLRELGVQPV VTLYHWDLPQ RLQDAYGGWA |
| 201 | NRALADHFRD YAELCFRHFG GQVKYWITID NPYVVAWHGY ATGRLAPGIR |
| 251 | GSPRLGYLVA HNLLLAHAKV WHLYNTSFRP TQGGQVSIAL SSHWINPRRM |
| 301 | TDHSIKECQK SLDFVLGWFA KPVFIDGDYP ESMKNNLSSI LPDFTESEKK |
| 351 | FIKGTADFFA LCFGPTLSFQ LLDPHMKFRQ LESPNLRQLL SWIDLEFNHP |
| 401 | QIFIVENGWF VSGTTKRDDA KYMYYLKKFI METLKAIKLD GVDVIGYTAW |
| 451 | SLMDGFEWHR GYSIRRGLFY VDFLSQDKML LPKSSALFYQ KLIEKNGFPP |
| 501 | LPENQPLEGT FPCDFAWGVV DNYIQVDTTL SQFTDLNVYL WDVHHSKRLI |
| 551 | KVDGVVTKKR KSYCVDFAAI QPQIALLQEM HVTHFRFSLD WALILPLGNQ |
| 601 | SQVNHTILQY YRCMASELVR VNITPVVALW QPMAPNQGLP RLLARQGAWE |
| 651 | NPYTALAFAE YARLCFQELG HHVKLWITMN EPYTRNMTYS AGHNLLKAHA |
| 701 | LAWHVYNEKF RHAQNGKISI ALQADWIEPA CPFSQKDKEV AERVLEFDIG |
| 751 | WLAEPIFGSG DYPWVMRDWL NQRNNFLLPY FTEDEKKLIQ GTFDFLALSH |
| 801 | YTTILVDSEK EDPIKYNDYL EVQEMTDITW LNSPSQVAW PWGLRKVLNW |
| 851 | LKFKYGDLPM YIISNGIDDG LHAEDDQLRV YYMQNYINEA LKAHILDGIN |
| 901 | LCGYFAYSFN DRTAPRFGLY RYAADQFEPK ASMKHYRKII DSNGFPGPET |
| 951 | LERFCPEEFT VCTECSFFHT RKSLGSGGGG SGGGGSGGGG SLKYPNASPL |
| 1001 | LGSSWGGLIH LYTATARNSY HLQIHKNGHV DGAPHQTIYS ALMIRSEDAG |
| 1051 | FVVITGVMSR RYLCMDFRGN IFGSHYFDPE NCRFQHQTLE NGYDVYHSPQ |
| 1101 | YHFLVSLGRA KRAFLPGMNP PPYSQFLSRR NEIPLIHFNT PIPRRHTQSA |
| 1151 | EDDSERDPLN VLKPRARMTP APASCSQELP SAEDNSPMAS DPLGVVRGGR |
| 1201 | VNTHAGGTGP EGCRPFAKFI * |

KL-D1-FGF23 (R179Q) amino acid sequence (SEQ ID NO: 21)

| | |
|---|---|
| 1 | MPASAPPRRP RPPPPSLSLL LVLLGLGGRR LRAEPGDGAQ TWARFSRPPA |
| 51 | PEAAGLFQGT FPDGFLWAVG SAAYQTEGGW QQHGKGASIW DTFTHHPLAP |
| 101 | PGDSRNASLP LGAPSPLQPA TGDVASDSYN NVFRDTEALR ELGVTHYRFS |
| 151 | ISWARVLPNG SAGVPNREGL RYYRRLLERL RELGVQPVVT LYHWDLPQRL |
| 201 | QDAYGGWANR ALADHFRDYA ELCFRHFGGQ VKYWITIDNP YVVAWHGYAT |
| 251 | GRLAPGIRGS PRLGYLVAHN LLLAHAKVWH LYNTSFRPTQ GGQVSIALSS |
| 301 | HWINPRRMTD HSIKECQKSL DFVLGWFAKP VFIDGDYPES MKNNLSSILP |
| 351 | DFTESEKKFI KGTADFFALC FGPTLSFQLL DPHMKFRQLE SPNLRQLLSW |
| 401 | IDLEFNHPQI FIVENGWFVS GTTKRDDAKY MYYLKKFIME TLKAIKLDGV |
| 451 | DVIGYTAWSL MDGFEWHRGY SIRRGLFYVD FLSQDKMLLP KSSALFYQKL |
| 501 | IEKNGFPPLP ENQPLEGSGG GGSGGGGSGG GGSLKYPNAS PLLGSSWGGL |
| 551 | IHLYTATARN SYHLQIHKNG HVDGAPHQTI YSALMIRSED AGFWITGVM |
| 601 | SRRYLCMDFR GNIFGSHYFD PENCRFQHQT LENGYDVYHS PQYHFLVSLG |
| 651 | RAKRAFLPGM NPPPYSQFLS RRNEIPLIHF NTPIPRRHTQ SAEDDSERDP |
| 701 | LNVLKPRARM TPAPASCSQE LPSAEDNSPM ASDPLGVVRG GRVNTHAGGT |
| 751 | GPEGCRPFAK FI* |

KL-D2-FGF23 (R179Q) amino acid sequence (SEQ ID NO: 22)

| | |
|---|---|
| 1 | MPASAPPRRP RPPPPSLSLL LVLLGLGGRR LPLPENQPLE GTFPCDFAWG |
| 51 | VVDNYIQVDT TLSQFTDLNV YLWDVHHSKR LIKVDGVVTK KRKSYCVDFA |
| 101 | AIQPQIALLQ EMHVTHFRFS LDWALILPLG NQSQVNHTIL QYYRCMASEL |
| 151 | VRVNITPVVA LWQPMAPNQG LPRLLARQGA WENPYTALAF AEYARLCFQE |
| 201 | LGHHVKLWIT MNEPYTRNMT YSAGHNLLKA HALAWHVYNE KFRHAQNGKI |
| 251 | SIALQADWIE PACPFSQKDK EVAERVLEFD IGWLAEPIFG SGDYPWVMRD |
| 301 | WLNQRNNFLL PYFTEDEKKL IQGTFDFLAL SHYTTILVDS EKEDPIKYND |
| 351 | YLEVQEMTDI TWLNSPSQVA VVPWGLRKVL NWLKFKYGDL PMYIISNGID |
| 401 | DGLHAEDDQL RVYYMQNYIN EALKAHILDG INLCGYFAYS FNDRTAPRFG |
| 451 | LYRYAADQFE PKASMKHYRK IIDSNGFPGP ETLERFCPEE FTVCTECSFF |
| 501 | HTRKSLGSGG GGSGGGGSGG GGSLKYPNAS PLLGSSWGGL IHLYTATARN |
| 551 | SYHLQIHKNG HVDGAPHQTI YSALMIRSED AGFVVITGVM SRRYLCMDFR |
| 601 | GNIFGSHYFD PENCRFQHQT LENGYDVYHS PQYHFLVSLG RAKRAFLPGM |
| 651 | NPPPYSQFLS RRNEIPLIHF NTPIPRRHTQ SAEDDSERDP LNVLKPRARM |
| 701 | TPAPASCSQE LPSAEDNSPM ASDPLGVVRG GRVNTHAGGT GPEGCRPFAK |
| 751 | FI* |

(KL-D1)2-FGF23 (R179Q) amino acid sequence (SEQ ID NO: 23)

| | |
|---|---|
| 1 | MPASAPPRRP RPPPPSLSLL LVLLGLGGRR LRAEPGDGAQ TWARFSRPPA |
| 51 | PEAAGLFQGT FPDGFLWAVG SAAYQTEGGW QQHGKGASIW DTFTHHPLAP |
| 101 | PGDSRNASLP LGAPSPLQPA TGDVASDSYN NVFRDTEALR ELGVTHYRFS |
| 151 | ISWARVLPNG SAGVPNREGL RYYRRLLERL RELGVQPVVT LYHWDLPQRL |
| 201 | QDAYGGWANR ALADHFRDYA ELCFRHFGGQ VKYWITIDNP YVVAWHGYAT |
| 251 | GRLAPGIRGS PRLGYLVAHN LLLAHAKVWH LYNTSFRPTQ GGQVSIALSS |
| 301 | HWINPRRMTD HSIKECQKSL DFVLGWFAKP VFIDGDYPES MKNNLSSILP |
| 351 | DFTESEKKFI KGTADFFALC FGPTLSFQLL DPHMKFRQLE SPNLRQLLSW |
| 401 | IDLEFNHPQI FIVENGWFVS GTTKRDDAKY MYYLKKFIME TLKAIKLDGV |
| 451 | DVIGYTAWSL MDGFEWHRGY SIRRGLFYVD FLSQDKMLLP KSSALFYQKL |
| 501 | IEKNGFPPLP ENQPLEGSGT FPDGFLWAVG SAAYQTEGGW QQHGKGASIW |
| 551 | DTFTHHPLAP PGDSRNASLP LGAPSPLQPA TGDVASDSYN NVFRDTEALR |
| 601 | ELGVTHYRFS ISWARVLPNG SAGVPNREGL RYYRRLLERL RELGVQPVVT |
| 651 | LYHWDLPQRL QDAYGGWANR ALADHFRDYA ELCFRHFGGQ VKYWITIDNP |
| 701 | YVVAWHGYAT GRLAPGIRGS PRLGYLVAHN LLLAHAKVWH LYNTSFRPTQ |
| 751 | GGQVSIALSS HWINPRRMTD HSIKECQKSL DFVLGWFAKP VFIDGDYPES |
| 801 | MKNNLSSILP DFTESEKKFI KGTADFFALC FGPTLSFQLL DPHMKFRQLE |
| 851 | SPNLRQLLSW IDLEFNHPQI FIVENGWFVS GTTKRDDAKY MYYLKKFIME |
| 901 | TLKAIKLDGV DVIGYTAWSL MDGFEWHRGY SIRRGLFYVD FLSQDKMLLP |
| 951 | KSSALFYQKL IEKNGFPEFG SGGGGSGGGG SGGGGSLKYP NASPLLGSSW |
| 1001 | GGLIHLYTAT ARNSYHLQIH KNGHVDGAPH QTIYSALMIR SEDAGFWIT |
| 1051 | GVMSRRYLCM DFRGNIFGSH YFDPENCRFQ HQTLENGYDV YHSPQYHFLV |
| 1101 | SLGRAKRAFL PGMNPPPYSQ FLSRRNEIPL IHFNTPIPRR HTQSAEDDSE |
| 1151 | RDPLNVLKPR ARMTPAPASC SQELPSAEDN SPMASDPLGV VRGGRVNTHA |
| 1201 | GGTGPEGCRP FAKFI* |

(KL-D2)2-FGF23 (R179Q) amino acid sequence (SEQ ID NO: 24)

| | |
|---|---|
| 1 | MPASAPPRRP RPPPPSLSLL LVLLGLGGRR LPLPENQPLE GTFPCDFAWG |
| 51 | VVDNYIQVDT TLSQFTDLNV YLWDVHHSKR LIKVDGVVTK KRKSYCVDFA |
| 101 | AIQPQIALLQ EMHVTHFRFS LDWALILPLG NQSQVNHTIL QYYRCMASEL |
| 151 | VRVNITPVVA LWQPMAPNQG LPRLLARQGA WENPYTALAF AEYARLCFQE |
| 201 | LGHHVKLWIT MNEPYTRNMT YSAGHNLLKA HALAWHVYNE KFRHAQNGKI |
| 251 | SIALQADWIE PACPFSQKDK EVAERVLEFD IGWLAEPIFG SGDYPWVMRD |
| 301 | WLNQRNNFLL PYFTEDEKKL IQGTFDFLAL SHYTTILVDS EKEDPIKYND |
| 351 | YLEVQEMTDI TWLNSPSQVA VVPWGLRKVL NWLKFKYGDL PMYIISNGID |
| 401 | DGLHAEDDQL RVYYMQNYIN EALKAHILDG INLCGYFAYS FNDRTAPRFG |
| 451 | LYRYAADQFE PKASMKHYRK IIDSNGFPGP ETLERFCPEE FTVCTECSFF |
| 501 | HTRKSLGTFP CDFAWGWDN YIQVDTTLSQ FTDLNVYLWD VHHSKRLIKV |
| 551 | DGWTKKRKS YCVDFAAIQP QIALLQEMHV THFRFSLDWA LILPLGNQSQ |
| 601 | VNHTILQYYR CMASELVRVN ITPVVALWQP MAPNQGLPRL LARQGAWENP |
| 651 | YTALAFAEYA RLCFQELGHH VKLWITMNEP YTRNMTYSAG HNLLKAHALA |
| 701 | WHVYNEKFRH AQNGKISIAL QADWIEPACP FSQKDKEVAE RVLEFDIGWL |
| 751 | AEPIFGSGDY PWVMRDWLNQ RNNFLLPYFT EDEKKLIQGT FDFLALSHYT |
| 801 | TILVDSEKED PIKYNDYLEV QEMTDITWLN SPSQVAVVPW GLRKVLNWLK |
| 851 | FKYGDLPMYI ISNGIDDGLH AEDDQLRVYY MQNYINEALK AHILDGINLC |
| 901 | GYFAYSFNDR TAPRFGLYRY AADQFEPKAS MKHYRKIIDS NGFGSGGGGS |
| 951 | GGGGSGGGGS LKYPNASPLL GSSWGGLIHL YTATARNSYH LQIHKNGHVD |
| 1001 | GAPHQTIYSA LMIRSEDAGF VVITGVMSRR YLCMDFRGNI FGSHYFDPEN |
| 1051 | CRFQHQTLEN GYDVYHSPQY HFLVSLGRAK RAFLPGMNPP PYSQFLSRRN |
| 1101 | EIPLIHFNTP IPRRHTQSAE DDSERDPLNV LKPRARMTPA PASCSQELPS |
| 1151 | AEDNSPMASD PLGWRGGRV NTHAGGTGPE GCRPFAKFI* |

FGF23 (R179Q)-Klotho extracellular domain amino acid sequence
(SEQ ID NO: 25)

| | |
|---|---|
| 1 | MLGARLRLWV CALCSVCSMS VLRAYPNASP LLGSSWGGLI HLYTATARNS |
| 51 | YHLQIHKNGH VDGAPHQTIY SALMIRSEDA GFWITGVMS RRYLCMDFRG |
| 101 | NIFGSHYFDP ENCRFQHQTL ENGYDVYHSP QYHFLVSLGR AKRAFLPGMN |
| 151 | PPPYSQFLSR RNEIPLIHFN TPIPRRHTQS AEDDSERDPL NVLKPRARMT |
| 201 | PAPASCSQEL PSAEDNSPMA SDPLGVVRGG RVNTHAGGTG PEGCRPFAKF |
| 251 | IGSGGGGSGG GGSGGGGSLK EPGDGAQTWA RFSRPPAPEA AGLFQGTFPD |
| 301 | GFLWAVGSAA YQTEGGWQQH GKGASIWDTF THHPLAPPGD SRNASLPLGA |
| 351 | PSPLQPATGD VASDSYNNVF RDTEALRELG VTHYRFSISW ARVLPNGSAG |
| 401 | VPNREGLRYY RRLLERLREL GVQPWTLYH WDLPQRLQDA YGGWANRALA |
| 451 | DHFRDYAELC FRHFGGQVKY WITIDNPYVV AWHGYATGRL APGIRGSPRL |
| 501 | GYLVAHNLLL AHAKVWHLYN TSFRPTQGGQ VSIALSSHWI NPRRMTDHSI |
| 551 | KECQKSLDFV LGWFAKPVFI DGDYPESMKN NLSSILPDFT ESEKKFIKGT |
| 601 | ADFFALCFGP TLSFQLLDPH MKFRQLESPN LRQLLSWIDL EFNHPQIFIV |
| 651 | ENGWFVSGTT KRDDAKYMYY LKKFIMETLK AIKLDGVDVI GYTAWSLMDG |
| 701 | FEWHRGYSIR RGLFYVDFLS QDKMLLPKSS ALFYQKLIEK NGFPPLPENQ |
| 751 | PLEGTFPCDF AWGWDNYIQ VDTTLSQFTD LNVYLWDVHH SKRLIKVDGV |
| 801 | VTKKRKSYCV DFAAIQPQIA LLQEMHVTHF RFSLDWALIL PLGNQSQVNH |
| 851 | TILQYYRCMA SELVRVNITP VVALWQPMAP NQGLPRLLAR QGAWENPYTA |
| 901 | LAFAEYARLC FQELGHHVKL WITMNEPYTR NMTYSAGHNL LKAHALAWHV |
| 951 | YNEKFRHAQN GKISIALQAD WIEPACPFSQ KDKEVAERVL EFDIGWLAEP |
| 1001 | IFGSGDYPWV MRDWLNQRNN FLLPYFTEDE KKLIQGTFDF LALSHYTTIL |
| 1051 | VDSEKEDPIK YNDYLEVQEM TDITWLNSPS QVAVVPWGLR KVLNWLKFKY |
| 1101 | GDLPMYIISN GIDDGLHAED DQLRVYYMQN YINEALKAHI LDGINLCGYF |
| 1151 | AYSFNDRTAP RFGLYRYAAD QFEPKASMKH YRKIIDSNGF PGPETLERFC |
| 1201 | PEEFTVCTEC SFFHTRKSL* |

FGF23 (R179Q)-KL-D1 amino acid sequence (SEQ ID NO: 26)

| | |
|---|---|
| 1 | MLGARLRLWV CALCSVCSMS VLRAYPNASP LLGSSWGGLI HLYTATARNS |
| 51 | YHLQIHKNGH VDGAPHQTIY SALMIRSEDA GFWITGVMS RRYLCMDFRG |
| 101 | NIFGSHYFDP ENCRFQHQTL ENGYDVYHSP QYHFLVSLGR AKRAFLPGMN |
| 151 | PPPYSQFLSR RNEIPLIHFN TPIPRRHTQS AEDDSERDPL NVLKPRARMT |
| 201 | PAPASCSQEL PSAEDNSPMA SDPLGVVRGG RVNTHAGGTG PEGCRPFAKF |
| 251 | IQGTFPDGFL WAVGSAAYQT EGGWQQHGKG ASIWDTFTHH PLAPPGDSRN |
| 301 | ASLPLGAPSP LQPATGDVAS DSYNNVFRDT EALRELGVTH YRFSISWARV |
| 351 | LPNGSAGVPN REGLRYYRRL LERLRELGVQ PVVTLYHWDL PQRLQDAYGG |
| 401 | WANRALADHF RDYAELCFRH FGGQVKYWIT IDNPYWAWH GYATGRLAPG |
| 451 | IRGSPRLGYL VAHNLLLAHA KVWHLYNTSF RPTQGGQVSI ALSSHWINPR |
| 501 | RMTDHSIKEC QKSLDFVLGW FAKPVFIDGD YPESMKNNLS SILPDFTESE |
| 551 | KKFIKGTADF FALCFGPTLS FQLLDPHMKF RQLESPNLRQ LLSWIDLEFN |
| 601 | HPQIFIVENG WFVSGTTKRD DAKYMYYLKK FIMETLKAIK LDGVDVIGYT |
| 651 | AWSLMDGFEW HRGYSIRRGL FYVDFLSQDK MLLPKSSALF YQKLIEKNGF |
| 652 | * |

FGF23 (R179Q)-KL-D₂ amino acid sequence (SEQ ID NO: 27)

| | |
|---|---|
| 1 | MLGARLRLWV CALCSVCSMS VLRAYPNASP LLGSSWGGLI HLYTATARNS |
| 51 | YHLQIHKNGH VDGAPHQTIY SALMIRSEDA GFWITGVMS RRYLCMDFRG |
| 101 | NIFGSHYFDP ENCRFQHQTL ENGYDVYHSP QYHFLVSLGR AKRAFLPGMN |
| 151 | PPPYSQFLSR RNEIPLIHFN TPIPRRHTQS AEDDSERDPL NVLKPRARMT |
| 201 | PAPASCSQEL PSAEDNSPMA SDPLGVVRGG RVNTHAGGTG PEGCRPFAKF |
| 251 | IGTFPCDFAW GVVDNYIQVD TTLSQFTDLN VYLWDVHHSK RLIKVDGVVT |
| 301 | KKRKSYCVDF AAIQPQIALL QEMHVTHFRF SLDWALILPL GNQSQVNHTI |
| 351 | LQYYRCMASE LVRVNITPVV ALWQPMAPNQ GLPRLLARQG AWENPYTALA |
| 401 | FAEYARLCFQ ELGHHVKLWI TMNEPYTRNM TYSAGHNLLK AHALAWHVYN |
| 451 | EKFRHAQNGK ISIALQADWI EPACPFSQKD KEVAERVLEF DIGWLAEPIF |
| 501 | GSGDYPli\JVMR DWLNQRNNFL LPYFTEDEKK LIQGTFDFLA LSHYTTILVD |
| 551 | SEKEDPIKYN DYLEVQEMTD ITWLNSPSQV AVVPWGLRKV LNWLKFKYGD |
| 601 | LPMYIISNGI DDGLHAEDDQ LRVYYMQNYI NEALKAHILD GINLCGYFAY |
| 651 | SFNDRTAPRF GLYRYAADQF EPKASMKHYR KIIDSNGF* |

FGF23 (R179Q)-(KL-D1)₂ amino acid sequence (SEQ ID NO: 28)

| | |
|---|---|
| 1 | MLGARLRLWV CALCSVCSMS VLRAYPNASP LLGSSWGGLI HLYTATARNS |
| 51 | YHLQIHKNGH VDGAPHQTIY SALMIRSEDA GFWITGVMS RRYLCMDFRG |
| 101 | NIFGSHYFDP ENCRFQHQTL ENGYDVYHSP QYHFLVSLGR AKRAFLPGMN |
| 151 | PPPYSQFLSR RNEIPLIHFN TPIPRRHTQS AEDDSERDPL NVLKPRARMT |
| 201 | PAPASCSQEL PSAEDNSPMA SDPLGVVRGG RVNTHAGGTG PEGCRPFAKF |
| 251 | IQGTFPDGFL WAVGSAAYQT EGGWQQHGKG ASIWDTFTHH PLAPPGDSRN |
| 301 | ASLPLGAPSP LQPATGDVAS DSYNNVFRDT EALRELGVTH YRFSISWARV |
| 351 | LPNGSAGVPN REGLRYYRRL LERLRELGVQ PVVTLYHWDL PQRLQDAYGG |
| 401 | WANRALADHF RDYAELCFRH FGGQVKYWIT IDNPYWAWH GYATGRLAPG |
| 451 | IRGSPRLGYL VAHNLLLAHA KVWHLYNTSF RPTQGGQVSI ALSSHWINPR |
| 501 | RMTDHSIKEC QKSLDFVLGW FAKPVFIDGD YPESMKNNLS SILPDFTESE |
| 551 | KKFIKGTADF FALCFGPTLS FQLLDPHMKF RQLESPNLRQ LLSWIDLEFN |
| 601 | HPQIFIVENG WFVSGTTKRD DAKYMYYLKK FIMETLKAIK LDGVDVIGYT |
| 651 | AWSLMDGFEW HRGYSIRRGL FYVDFLSQDK MLLPKSSALF YQKLIEKNGF |
| 701 | QGTFPDGFLW AVGSAAYQTE GGWQQHGKGA SIWDTFTHHP LAPPGDSRNA |
| 751 | SLPLGAPSPL QPATGDVASD SYNNVFRDTE ALRELGVTHY RFSISWARVL |
| 801 | PNGSAGVPNR EGLRYYRRLL ERLRELGVQP VVTLYHWDLP QRLQDAYGGW |
| 851 | ANRALADHFR DYAELCFRHF GGQVKYWITI DNPYVVAWHG YATGRLAPGI |
| 901 | RGSPRLGYLV AHNLLLAHAK VWHLYNTSFR PTQGGQVSIA LSSHWINPRR |
| 951 | MTDHSIKECQ KSLDFVLGWF AKPVFIDGDY PESMKNNLSS ILPDFTESEK |
| 1001 | KFIKGTADFF ALCFGPTLSF QLLDPHMKFR QLESPNLRQL LSWIDLEFNH |
| 1051 | PQIFIVENGW FVSGTTKRDD AKYMYYLKKF IMETLKAIKL DGVDVIGYTA |
| 1101 | WSLMDGFEWH RGYSIRRGLF YVDFLSQDKM LLPKSSALFY QKLIEKNGF* |

FGF23 (R179Q)-(KL-D2)₂ amino acid sequence (SEQ ID NO: 29)

| | |
|---|---|
| 1 | MLGARLRLWV CALCSVCSMS VLRAYPNASP LLGSSWGGLI HLYTATARNS |
| 51 | YHLQIHKNGH VDGAPHQTIY SALMIRSEDA GFWITGVMS RRYLCMDFRG |
| 101 | NIFGSHYFDP ENCRFQHQTL ENGYDVYHSP QYHFLVSLGR AKRAFLPGMN |
| 151 | PPPYSQFLSR RNEIPLIHFN TPIPRRHTQS AEDDSERDPL NVLKPRARMT |
| 201 | PAPASCSQEL PSAEDNSPMA SDPLGVVRGG RVNTHAGGTG PEGCRPFAKF |
| 251 | IGTFPCDFAW GVVDNYIQVD TTLSQFTDLN VYLWDVHHSK RLIKVDGVVT |
| 301 | KKRKSYCVDF AAIQPQIALL QEMHVTHFRF SLDWALILPL GNQSQVNHTI |
| 351 | LQYYRCMASE LVRVNITPVV ALWQPMAPNQ GLPRLLARQG AWENPYTALA |
| 401 | FAEYARLCFQ ELGHHVKLWI TMNEPYTRNM TYSAGHNLLK AHALAWHVYN |
| 451 | EKFRHAQNGK ISIALQADWI EPACPFSQKD KEVAERVLEF DIGWLAEPIF |
| 501 | GSGDYPli\JVMR DWLNQRNNFL LPYFTEDEKK LIQGTFDFLA LSHYTTILVD |
| 551 | SEKEDPIKYN DYLEVQEMTD ITWLNSPSQV AVVPWGLRKV LNWLKFKYGD |
| 601 | LPMYIISNGI DDGLHAEDDQ LRVYYMQNYI NEALKAHILD GINLCGYFAY |
| 651 | SFNDRTAPRF GLYRYAADQF EPKASMKHYR KIIDSNGFGT FPCDFAWGVV |
| 701 | DNYIQVDTTL SQFTDLNVYL WDVHHSKRLI KVDGVVTKKR KSYCVDFAAI |
| 751 | QPQIALLQEM HVTHFRFSLD WALILPLGNQ SQVNHTILQY YRCMASELVR |
| 801 | VNITPWALW QPMAPNQGLP RLLARQGAWE NPYTALAFAE YARLCFQELG |
| 851 | HHVKLWITMN EPYTRNMTYS AGHNLLKAHA LAWHVYNEKF RHAQNGKISI |
| 901 | ALQADWIEPA CPFSQKDKEV AERVLEFDIG WLAEPIFGSG DYPWVMRDWL |
| 951 | NQRNNFLLPY FTEDEKKLIQ GTFDFLALSH YTTILVDSEK EDPIKYNDYL |
| 1001 | EVQEMTDITW LNSPSQVAVV PWGLRKVLNW LKFKYGDLPM YIISNGIDDG |
| 1051 | LHAEDDQLRV YYMQNYINEA LKAHILDGIN LCGYFAYSFN DRTAPRFGLY |
| 1101 | RYAADQFEPK ASMKHYRKI I DSNGF* |

FGF19 nucleic acid sequence (NM_005117) (SEQ ID NO: 30)
Protein coding region (464-1114)

| | |
|---|---|
| 1 | gctcccagcc aagaacctcg gggccgctgc gcggtgggga ggagttcccc gaaacccggc |
| 61 | cgctaagcga ggcctcctcc tcccgcagat ccgaacggcc tgggcggggt caccccggct |
| 121 | gggacaagaa gccgccgcct gcctgcccgg gcccggggag ggggctgggg ctggggccgg |
| 181 | aggcggggtg tgagtgggtg tgtgcggggg gcggaggctt gatgcaatcc cgataagaaa |
| 241 | tgctcgggtg tcttgggcac ctacccgtgg ggcccgtaag gcgctactat ataaggctgc |
| 301 | cggcccggag ccgccgcgcc gtcagagcag gagcgctgcg tccaggatct agggccacga |
| 361 | ccatcccaac ccggcactca cagccccgca gcgcatcccg gtcgccgccc agcctcccgc |
| 421 | acccccatcg ccggagctgc gccgagagcc ccagggaggt gccatgcgga gcgggtgtgt |
| 481 | ggtggtccac gtatggatcc tggccggcct ctggctggcc gtggccgggc gccccctcgc |
| 541 | cttctcggac gcggggcccc acgtgcacta cggctggggc gaccccatcc gcctgcggca |
| 601 | cctgtacacc tccggccccc acgggctctc cagctgcttc ctgcgcatcc gtgccgacgg |
| 661 | cgtcgtggac tgcgcgcggg gccagagcgc gcacagtttg ctggagatca aggcagtcgc |
| 721 | tctgcggacc gtggccatca agggcgtgca cagcgtgcgg tacctctgca tgggcgccga |
| 781 | cggcaagatg caggggctgc ttcagtactc ggaggaagac tgtgctttcg aggaggagat |
| 841 | ccgcccagat ggctacaatg tgtaccgatc cgagaagcac cgcctcccgg tctccctgag |
| 901 | cagtgccaaa cagcggcagc tgtacaagaa cagaggcttt cttccactct ctcatttcct |
| 961 | gcccatgctg cccatggtcc cagaggagcc tgaggacctc aggggccact tggaatctga |
| 1021 | catgttctct tcgcccctgg agaccgacag catggaccca tttgggcttg tcaccggact |
| 1081 | ggaggccgtg aggagtccca gctttgagaa gtaactgaga ccatgcccgg gcctcttcac |
| 1141 | tgctgccagg ggctgtggta cctgcagcgt gggggacgtg cttctacaag aacagtcctg |
| 1201 | agtccacgtt ctgtttagct ttaggaagaa acatctagaa gttgtacata ttcagagttt |
| 1261 | tccattggca gtgccagttt ctagccaata gacttgtctg atcataacat tgtaagcctg |
| 1321 | tagcttgccc agctgctgcc tgggccccca ttctgctccc tcgaggttgc tggacaagct |
| 1381 | gctgcactgt ctcagttctg cttgaatacc tccatcgatg gggaactcac ttcctttgga |
| 1441 | aaaattctta tgtcaagctg aaattctcta attttttctc atcacttccc caggagcagc |
| 1501 | cagaagacag gcagtagttt taatttcagg aacaggtgat ccactctgta aaacagcagg |
| 1561 | taaatttcac tcaaccccat gtgggaattg atctatatct ctacttccag ggaccatttg |
| 1621 | cccttcccaa atccctccag gccagaactg actggagcag gcatggccca ccaggcttca |
| 1681 | ggagtagggg aagcctggag ccccactcca gccctgggac aacttgagaa ttccccctga |
| 1741 | ggccagttct gtcatggatg ctgtcctgag aataacttgc tgtcccggtg tcacctgctt |
| 1801 | ccatctccca gcccaccagc cctctgccca cctcacatgc ctccccatgg attggggcct |
| 1861 | cccaggcccc ccaccttatg tcaacctgca cttcttgttc aaaaatcagg aaaagaaaag |
| 1921 | atttgaagac cccaagtctt gtcaataact tgctgtgtgg aagcagcggg ggaagaccta |
| 1981 | gaaccctttc cccagcactt ggttttccaa catgatattt atgagtaatt tattttgata |
| 2041 | tgtacatctc ttattttctt acattattta tgcccccaaa ttatatttat gtatgtaagt |
| 2101 | gaggtttgtt ttgtatatta aaatggagtt tgtttgtaaa aaaaaaaaaa aaaaaaa |

FGF19 amino acid sequence (NP_005108) (SEQ ID NO: 31)

| | |
|---|---|
| 1 | MRSGCWV1-N WILAGLWLAV AGRPLAFSDA GHNHYGWGD PIRLRHLYTS GPHGLSSCFL |
| 61 | RIRADGVVDC ARGQSAHSLL EIKAVALRTV AIKGVHSVRY LCMGADGKMQ GLLQYSEEDC |
| 121 | AFEEEIRPDG YNVYRSEKHR LPVSLSSAKQ RQLYKNRGFL PLSHFLPMLP MVPEEPEDLR |
| 181 | GHLESDMFSS PLETDSMDPF GLVTGLEAVR SPSFEK |
| FGF21 | nucleic acid sequence (NM_019113) (SEQ ID NO: 32) |

Protein coding region 151-780

| | |
|---|---|
| 1 | CTGTCAGCTG AGGATCCAGC CGAAAGAGGA GCCAGGCACT CAGGCCACCT GAGTCTACTC |
| 61 | ACCTGGACAA CGGAATCTG GCACCAATTC TAAACCACTC AGCTTCTCCG AGCTCACACC |
| 121 | CCGGAGATCA CCTGAGGACC CGAGCCATTG ATGGACTCGG ACGAGACCGG GTTCGAGCAC |
| 181 | TCAGGACTGT GGGTTTCTGT GCTGGCTGGT CTTCTGCTGG GAGCCTGCCA GGCACACCCC |
| 241 | ATCCCTGACT CCAGTCCTCT CCTGCAATTC GGGGGCCAAG TCCGGCAGCG GTACCTCTAC |
| 301 | ACAGATGATG CCCAGCAGAC AGAAGCCCAC CTGGAGATCA GGGAGGATGG GACGGTGGGG |
| 361 | GGCGCTGCTG ACCAGAGCCC CGAAAGTCTC CTGCAGCTGA AAGCCTTGAA GCCGGGAGTT |
| 421 | ATTCAAATCT TGGGAGTCAA GACATCCAGG TTCCTGTGCC AGCGGCCAGA TGGGGCCCTG |
| 481 | TATGGATCGC TCCACTTTGA CCCTGAGGCC TGCAGCTTCC GGGAGCTGCT TCTTGAGGAC |
| 541 | GGATACAATG TTTACCAGTC CGAAGCCCAC GGCCTCCCGC TGCACCTGCC AGGGAACAAG |
| 601 | TCCCCACACC GGGACCCTGC ACCCCGAGGA CCAGCTCGCT TCCTGCCACT ACCAGGCCTG |
| 661 | CCCCCCGCAC TCCCGGAGCC ACCCGGAATC CTGGCCCCCC AGCCCCCCGA TGTGGGCTCC |
| 721 | TCGGACCCTC TGAGCATGGT GGGACCTTCC CAGGGCCGAA GCCCCAGCTA CGCTTCCTGA |
| 781 | AGCCAGAGGC TGTTTACTAT GACATCTCCT CTTTATTTAT TAGGTTATTT ATCTTATTTA |
| 841 | TTTTTTTATT TTTCTTACTT GAGATAATAA AGAGTTCCAG AGGAGAAAAA AAAAAAAAAA |
| 901 | AAAAAAAAAA AAAAAAAAAA AAAAAAAAAA AAAAAAAAAA |

FGF21 amino acid sequence (NP_061986) (SEQ ID NO: 33)

| | |
|---|---|
| 1 | MDSDETGFEH SGLWVSVLAG LLLGACQAHP IPDSSPLLQF GGQVRQRYLY TDDAQQTEAH |
| 61 | LEIREDGTVG GAADQSPESL LQLKALKPGV IQILGVKTSR FLCQRPDGAL YGSLHFDPEA |
| 121 | CSFRELLLED GYNVYQSEAH GLPLHLPGNK SPHRDPAPRG PARFLPLPGL PPALPEPPGI |
| 181 | LAPQPPDVGS SDPLSMVGPS QGRSPSYAS |

FGF23 nucleic acid sequence (NM_020638) (SEQ ID NO: 34)
Protein coding region 147-902

| | |
|---|---|
| 1 | cggcaaaaag gagggaatcc agtctaggat cctcacacca gctacttgca agggagaagg |
| 61 | aaaaggccag taaggcctgg gccaggagag tcccgacagg agtgtcaggt ttcaatctca |
| 121 | gcaccagcca ctcagagcag ggcacgatgt tgggggcccg cctcaggctc tgggtctgtg |
| 181 | ccttgtgcag cgtctgcagc atgagcgtcc tcagagccta tcccaatgcc tccccactgc |
| 241 | tcggctccag ctggggtggc ctgatccacc tgtacacagc cacagccagg aacagctacc |
| 301 | acctgcagat ccacaagaat ggccatgtgg atggcgcacc ccatcagacc atctacagtg |
| 361 | ccctgatgat cagatcagag gatgctggct ttgtggtgat tacaggtgtg atgagcagaa |
| 421 | gatacctctg catggatttc agaggcaaca tttttggatc acactatttc gacccggaga |
| 481 | actgcaggtt ccaacaccag acgctggaaa acgggtacga cgtctaccac tctcctcagt |
| 541 | atcacttcct ggtcagtctg ggccgggcga agagagcctt cctgccaggc atgaacccac |
| 601 | ccccgtactc ccagttcctg tcccggagga acgagatccc cctaattcac ttcaacaccc |
| 661 | ccataccacg gcggcacacc cggagcgccg aggacgactc ggagcgggac cccctgaacg |
| 721 | tgctgaagcc ccgggcccgg atgaccccgg ccccggcctc ctgttcacag gagctcccga |
| 781 | gcgccgagga caacagcccg atggccagtg acccattagg ggtggtcagg ggcggtcgag |
| 841 | tgaacacgca cgctggggga acgggcccgg aaggctgccg ccccttcgcc aagttcatct |
| 901 | agggtcgctg gaagggcacc ctctttaacc catccctcag caaacgcagc tcttcccaag |
| 961 | gaccaggtcc cttgacgttc cgaggatggg aaaggtgaca ggggcatgta tggaatttgc |
| 1021 | tgcttctctg gggtcccttc cacaggaggt cctgtgagaa ccaacctttg aggcccaagt |
| 1081 | catggggttt caccgccttc ctcactccat atagaacacc tttcccaata ggaaacccca |
| 1141 | acaggtaaac tagaaatttc cccttcatga aggtagagag aaggggtctc tcccaacata |
| 1201 | tttctcttcc ttgtgcctct cctctttatc acttttaagc ataaaaaaaa aaaaaaaaaa |
| 1261 | aaaaaaaaaa aaaagcagtg ggttcctgag ctcaagactt tgaaggtgta gggaagagga |
| 1321 | aatcggagat cccagaagct tctccactgc cctatgcatt tatgttagat gccccgatcc |
| 1381 | cactggcatt tgagtgtgca aaccttgaca ttaacagctg aatggggcaa gttgatgaaa |
| 1441 | acactacttt caagccttcg ttcttccttg agcatctctg gggaagagct gtcaaaagac |
| 1501 | tggtggtagg ctggtgaaaa cttgacagct agacttgatg cttgctgaaa tgaggcagga |
| 1561 | atcataatag aaaactcagc ctccctacag ggtgagcacc ttctgtctcg ctgtctccct |
| 1621 | ctgtgcagcc acagccagag ggcccagaat ggccccactc tgttcccaag cagttcatga |
| 1681 | tacagcctca ccttttggcc ccatctctgg tttttgaaaa tttggtctaa ggaataaata |
| 1741 | gcttttacac tggctcacga aaatctgccc tgctagaatt tgcttttcaa aatggaaata |
| 1801 | aattccaact ctcctaagag gcatttaatt aaggctctac ttccaggttg agtaggaatc |
| 1861 | cattctgaac aaactacaaa aatgtgactg ggaagggggc tttgagagac tgggactgct |
| 1921 | ctgggttagg ttttctgtgg actgaaaaat cgtgtccttt tctctaaatg aagtggcatc |
| 1981 | aaggactcag ggggaaagaa atcaggggac atgttataga agttatgaaa agacaaccac |
| 2041 | atggtcaggc tcttgtctgt ggtctctagg gctctgcagc agcagtggct cttcgattag |
| 2101 | ttaaaactct cctaggctga cacatctggg tctcaatccc cttggaaatt cttggtgcat |
| 2161 | taaatgaagc cttaccccat tactgcggtt cttcctgtaa gggggctcca ttttcctccc |
| 2221 | tctctttaaa tgaccaccta aaggacagta tattaacaag caaagtcgat tcaacaacag |
| 2281 | cttcttccca gtcacttttt tttttctcac tgccatcaca tactaacctt atactttgat |
| 2341 | ctattctttt tggttatgag agaaatgttg ggcaactgtt tttacctgat ggttttaagc |
| 2401 | tgaacttgaa ggactggttc ctattctgaa acagtaaaac tatgtataat agtatatagc |
| 2461 | catgcatggc aaatatttta atatttctgt tttcatttcc tgttggaaat attatcctgc |
| 2521 | ataatagcta ttggaggctc ctcagtgaaa gatcccaaaa ggattttggt ggaaaactag |
| 2581 | ttgtaatctc acaaactcaa cactaccatc aggggttttc tttatggcaa agccaaaata |
| 2641 | gctcctacaa tttcttatat ccctcgtcat gtggcagtat ttatttattt atttggaagt |
| 2701 | ttgcctatcc ttctatattt atagatattt ataaaaatgt aacccctttt tcctttcttc |
| 2761 | tgtttaaaat aaaaataaaa tttatctcag cttctgttag cttatcctct ttgtagtact |
| 2821 | acttaaaagc atgtcggaat ataagaataa aaaggattat gggaggggaa cattagggaa |
| 2881 | atccagagaa ggcaaaattg aaaaaaagat tttagaattt taaaattttc aaagatttct |
| 2941 | tccattcata aggagactca atgattttaa ttgatctaga cagaattatt taagttttat |
| 3001 | caatattgga tttctggt |

FGF23 amino acid sequence (NP_065689) (SEQ ID NO: 35)

| | |
|---|---|
| 1 | MLGARLRLWV CALCSVCSMS VLRAYPNASP LLGSSWGGLI HLYTATARNS YHLQIHKNGH |
| 61 | VDGAPHQTIY SALMIRSEDA GFVVITGVMS RRYLCMDFRG NIFGSHYFDP ENCRFQHQTL |
| 121 | ENGYDVYHSP QYHFLVSLGR AKRAFLPGMN PPPYSQFLSR RNEIPLIHFN TPIPRRHTRS |
| 181 | AEDDSERDPL NVLKPRARMT PAPASCSQEL PSAEDNSPMA SDPLGVVRGG RVNTHAGGTG |
| 241 | PEGCRPFAKF I |

FGF23 (R179Q) amino acid sequence (SEQ ID NO: 36)

| | |
|---|---|
| 1 | MLGARLRLWV CALCSVCSMS VLRAYPNASP LLGSSWGGLI HLYTATARNS YHLQIHKNGH |
| 61 | VDGAPHQTIY SALMIRSEDA GFVVITGVMS RRYLCMDFRG NIFGSHYFDP ENCRFQHQTL |
| 121 | ENGYDVYHSP QYHFLVSLGR AKRAFLPGMN PPPYSQFLSR RNEIPLIHFN TPIPRRHTQS |
| 181 | AEDDSERDPL NVLKPRARMT PAPASCSQEL PSAEDNSPMA SDPLGVVRGG RVNTHAGGTG |
| 241 | PEGCRPFAKF I |

Human beta-Klotho domain 1 (b-KL-D1) amino acid sequence (SEQ ID NO: 37)

| | |
|---|---|
| 77 | ydt fpknffwgig tgalqvegsw kkdgkgpsiw dhfihthlkn |
| 121 | vsstngssds yiflekdlsa ldfigvsfyq fsiswprlfp dgivtvanak glqyystlld |
| 181 | alvlrniepi vtlyhwdlpl alqekyggwk ndtildifnd yatycfqmfg drvkywitih |
| 241 | npylvawhgy gtgmhapgek gnlaavytvg hnlikahskv whnynthfrp hqkgwlsitl |
| 301 | gshwiepnrs entmdifkcq qsmvsvlgwf anpihgdgdy pegmrkklfs vlpifseaek |
| 361 | hemrgtadff afsfgpnnfk pintmakmgq nvslnlreal nwikleynnp rillaengwf |
| 421 | tdsrvktedt talymmknfl sqvlqairld eirvfgytaw slldgfewqd aytirrglfy |
| 481 | vdfnskqker kpkssahyyk qiirengf |

Human beta-Klotho domain 2 (b-KL-D2) amino acid sequence (SEQ ID NO: 38)

| | |
|---|---|
| 571 | trpaqctdfv nikkqlemla rmkvthyrfa |
| 601 | ldwasvlptg nlsavnrqal ryyrcvvseg lklgisamvt lyypthahlg lpepllhadg |
| 661 | wlnpstaeaf qayaglcfqe lgdlvklwit inepnrlsdi ynrsgndtyg aahnllvaha |
| 721 | lawrlydrqf rpsqrgaysl slhadwaepa npyadshwra aerflqfela wfaeplfktg |
| 781 | dypaamreyi askhrrglss salprlteae rrllkgtvdf calnhfttrf vmheqlagsr |
| 841 | ydsdrdigfl gditrlsspt rlavipwgvr kllrwvrrny gdmdlyitas giddgaledd |
| 901 | rlrkyylgky lgevlkayll dkvrikgyya fklaeekskp rfgfftsdfk akssiqfynk |
| 961 | vissrgf |

Beta-Klotho extracellular domain (without signal peptide) amino acid
sequence (SEQ ID NO: 39)

| | |
|---|---|
| 52 | gfsgdgral |
| 61 | wsknpnftpv nesqlflydt fpknffwgig tgalqvegsw kkdgkgpsiw dhfihthlkn |
| 121 | vsstngssds yiflekdlsa ldfigvsfyg fsiswprlfp dgivtvanak glqyystlld |
| 181 | alvlrniepi vtlyhwdlpl alqekyggwk ndtildifnd yatycfqmfg drvkywitih |
| 241 | npylvawhgy gtgmhapgek gnlaavytvg hnlikahskv whnynthfrp hqkgwlsitl |
| 301 | gshwiepnrs entmdifkcg gsmvsvlgwf anpihgdgdy pegmrkklfs vlpifseaek |
| 361 | hemrgtadff afsfgpnnfk pintmakmgq nvslnlreal nwikleynnp rillaengwf |
| 421 | tdsrvktedt talymmknfl sqvlgairld eirvfgytaw slldgfewqd aytirrglfy |
| 481 | vdfnskqker kpkssahyyk glirengfsl kestpdvggq fpcdfswgvt esvlkpesva |
| 541 | sspqfsdphl yvwnatgnrl lhrvegvrlk trpagctdfv nikkglemla rmkvthyrfa |
| 601 | ldwasvlptg nlsavnrgal ryyrcvvseg lklgisamvt lyypthahlg lpepllhadg |
| 661 | wlnpstaeaf gayaglcfge lgdlvklwit inepnrlsdi ynrsgndtyg aahnllvaha |
| 721 | lawrlydrqf rpsgrgaysl slhadwaepa npyadshwra aerflgfela wfaeplfktg |
| 781 | dypaamreyi askhrrglss salprlteae rrllkgtvdf calnhfttrf vmheqlagsr |
| 841 | ydsdrdigfl gditrlsspt rlavipwgvr kllrwvrrny gdmdlyitas giddgaledd |
| 901 | rlrkyylgky lgevlkayll dkvrikgyya fklaeekskp rfgfftsdfk akssiqfynk |
| 961 | vissrgfpfe nsssrcsgtg entectvclf lvqkkpl |

sKlotho without signal peptide-FGF23 amino acid sequence (without signal
peptide) (SEQ ID NO: 40)

| | |
|---|---|
| | EPGDGAQ TWARFSRPPA |
| 51 | PEAAGLFQGT FPDGFLWAVG SAAYQTEGGW QQHGKGASIW DTFTHHPLAP |
| 101 | PGDSRNASLP LGAPSPLQPA TGDVASDSYN NVFRDTEALR ELGVTHYRFS |
| 151 | ISWARVLPNG SAGVPNREGL RYYRRLLERL RELGVQPVVT LYHWDLPQRL |
| 201 | QDAYGGWANR ALADHFRDYA ELCFRHFGGQ VKYWITIDNP YVVAWHGYAT |
| 251 | GRLAPGIRGS PRLGYLVAHN LLLAHAKVWH LYNTSFRPTQ GGQVSIALSS |
| 301 | HWINPRBMTD HSIKECQKSL DFVLGWFAKP VFIDGDYPES MKNNLSSILP |
| 351 | DFTESEKKFI KGTADFFALC FGPTLSFQLL DPHMKFRQLE SPNLRQLLSW |
| 401 | IDLEFNHPQI FIVENGWFVS GTTKRDDAKY MYYLKKFIME TLKAIKLDGV |
| 451 | DVIGYTAWSL MDGFEWHRGY SIRRGLFYVD FLSQDKMLLP KSSALFYQKL |
| 501 | IEKNGFPPLP ENQPLEGTFP CDFAWGVVDN YIQVDTTLSQ FTDLNVYLWD |
| 551 | VHHSKRLIKV DGVVTKKRKS YCVDFAAIQP QIALLQEMHV THFRFSLDWA |
| 601 | LILPLGNQSQ VNHTILQYYR CMASELVRVN ITPVVALWQP MAPNQGLPRL |
| 651 | LARQGAWENP YTALAFAEYA RLCFQELGHH VKLWITMNEP YTRNMTYSAG |
| 701 | HNLLKAHALA WHVYNEKFRH AQNGKISIAL QADWIEPACP FSQKDKEVAE |
| 751 | RVLEFDIGWL AEPIFGSGDY PWVMRDWLNQ RNNFLLPYFT EDEKKLIQGT |
| 801 | FDFLALSHYT TILVDSEKED PIKYNDYLEV QEMTDITWLN SPSQVAVVPW |
| 851 | GLRKVLNWLK FKYGDLPMYI ISNGIDDGLH AEDDQLRVYY MQNYINEALK |
| 901 | AHILDGINLC GYFAYSFNDR TAPRFGLYRY AADQFEPKAS MKHYRKIIDS |
| 951 | NGFPGPETLE RFCPEEFTVC TECSFFHTRK SLGSGGGGSG GGGSGGGGSL |
| 1001 | KYPNASPLLG SSWGGLIHLY TATARNSYHL QIHKNGHVDG APHQTIYSAL |
| 1051 | MIRSEDAGFV VITGVMSRRY LCMDFRGNIF GSHYFDPENC RFQHQTLENG |
| 1101 | YDVYHSPQYH FLVSLGRAKR AFLPGMNPPP YSQFLSRRNE IPLIHFNTPI |
| 1151 | PRRHTRSAED DSERDPLNVL KPRARMTPAP ASCSQELPSA EDNSPMASDP |
| 1201 | LGVVRGGRVN THAGGTGPEG CRPFAKFI* |

sKlotho without signal pepfide-FGF23 (R179C) (without signal peptide)
amino acid sequence (SEQ ID NO: 41)

| | |
|---|---|
| | EPGDGAQ TWARFSRPPA |
| 51 | PEAAGLFQGT FPDGFLWAVG SAAYQTEGGW QQHGKGASIW DTFTHHPLAP |
| 101 | PGDSRNASLP LGAPSPLQPA TGDVASDSYN NVFRDTEALR ELGVTHYRFS |
| 151 | ISWARVLPNG SAGVPNREGL RYYRRLLERL RELGVQPVVT LYHWDLPQRL |
| 201 | QDAYGGWANR ALADHFRDYA ELCFRHFGGQ VKYWITIDNP YVVAWHGYAT |
| 251 | GRLAPGIRGS PRLGYLVAHN LLLAHAKVWH LYNTSFRPTQ GGQVSIALSS |
| 301 | HWINPRPMTD HSIKECQKSL DFVLGWFAKP VFIDGDYPES MKNNLSSILP |
| 351 | DFTESEKKFI KGTADFFALC FGPTLSFQLL DPHMKFRQLE SPNLRQLLSW |
| 401 | IDLEFNHPQI FIVENGWFVS GTTKRDDAKY MYYLKKFIME TLKAIKLDGV |
| 451 | DVIGYTAWSL MDGFEWHRGY SIRRGLFYVD FLSQDKMLLP KSSALFYQKL |
| 501 | IEKNGFPPLP ENQPLEGTFP CDFAWGVVDN YIQVDTTLSQ FTDLNVYLWD |
| 551 | VHHSKRLIKV DGVVTKKRKS YCVDFAAIQP QIALLQEMHV THFRFSLDWA |
| 601 | LILPLGNQSQ VNHTILQYYR CMASELVRVN ITPVVALWQP MAPNQGLPRL |
| 651 | LARQGAWENP YTALAFAEYA RLCFQELGHH VKLWITMNEP YTRNMTYSAG |
| 701 | HNLLKAHALA WHVYNEKFRH AQNGKISIAL QADWIEPACP FSQKDKEVAE |
| 751 | RVLEFDIGWL AEPIFGSGDY PWVMRDWLNQ RNNFLLPYFT EDEKKLIQGT |
| 801 | FDFLALSHYT TILVDSEKED PIKYNDYLEV QEMTDITWLN SPSQVAWPW |
| 851 | GLRKVLNWLK FKYGDLPMYI ISNGIDDGLH AEDDQLRVYY MQNYINEALK |
| 901 | AHILDGINLC GYFAYSFNDR TAPRFGLYRY AADQFEPKAS MKHYRKIIDS |
| 951 | NGFPGPETLE RFCPEEFTVC TECSFFHTRK SLGSGGGGSG GGGSGGGGSL |
| 1001 | KYPNASPLLG SSWGGLIHLY TATARNSYHL QIHKNGHVDG APHQTIYSAL |
| 1051 | MIRSEDAGFV VITGVMSRRY LCMDFRGNIF GSHYFDPENC RFQHQTLENG |
| 1101 | YDVYHSPQYH FLVSLGRAKR AFLPGMNPPP YSQFLSRRNE IPLIHFNTPI |
| 1151 | PRRHTQSAED DSERDPLNVL KPRARMTPAP ASCSQELPSA EDNSPMASDP |
| 1201 | LGWRGGRVN THAGGTGPEG CRPFAKFI* |

FGF23 without signal peptide (SEQ ID NO: 42)

| | |
|---|---|
| | YPNASP LLGSSWGGLI HLYTATARNS YHLQIHKNGH |
| 61 | VDGAPHQTIY SALMIRSEDA GFVVITGVMS RRYLCMDFRG NIFGSHYFDP ENCRFQHQTL |
| 121 | ENGYDVYHSP QYHFLVSLGR AKRAFLPGMN PPPYSQFLSR RNEIPLIHFN TPIPRRHTRS |
| 181 | AEDDSERDPL NVLKPRARMT PAPASCSQEL PSAEDNSPMA SDPLGVVRGG RVNTHAGGTG |
| 241 | PEGCRPFAKF I |

FGF23(R179Q) without signal peptide (SEQ ID NO: 43)

| | |
|---|---|
| | YPNASP LLGSSWGGLI HLYTATARNS YHLQIHKNGH |
| 61 | VDGAPHQTIY SALMIRSEDA GFVVITGVMS RRYLCMDFRG NIFGSHYFDP ENCRFQHQTL |
| 121 | ENGYDVYHSP QYHFLVSLGR AKRAFLPGMN PPPYSQFLSR RNEIPLIHFN TPIPRRHTQS |
| 181 | AEDDSERDPL NVLKPRARMT PAPASCSQEL PSAEDNSPMA SDPLGVVRGG RVNTHAGGTG |
| 241 | PEGCRPFAKF I |

sKlotho with Moth-signal peptide (SEQ ID NO: 44)

| | |
|---|---|
| 1 | MPASAPPRRP RPPPPSLSLL LVLLGLGGRR LRAEPGDGAQ TWARFSRPPA |
| 51 | PEAAGLFQGT FPDGFLWAVG SAAYQTEGGW QQHGKGASIW DTFTHHPLAP |
| 101 | PGDSRNASLP LGAPSPLQPA TGDVASDSYN NVFRDTEALR ELGVTHYRFS |
| 151 | ISWARVLPNG SAGVPNREGL RYYRRLLERL RELGVQPVVT LYHWDLPQRL |
| 201 | QDAYGGWANR ALADHFRDYA ELCFRHFGGQ VKYWITIDNP YVVAWHGYAT |
| 251 | GRLAPGIRGS PRLGYLVAHN LLLAHAKVWH LYNTSFRPTQ GGQVSIALSS |
| 301 | HWINPRRMTD HSIKECQKSL DFVLGWFAKP VFIDGDYPES MKNNLSSILP |
| 351 | DFTESEKKFI KGTADFFALC FGPTLSFQLL DPHMKFRQLE SPNLRQLLSW |
| 401 | IDLEFNHPQI FIVENGWFVS GTTKRDDAKY MYYLKKFIME TLKAIKLDGV |
| 451 | DVIGYTAWSL MDGFEWHRGY SIRRGLFYVD FLSQDKMLLP KSSALFYQKL |
| 501 | IEKNGFPPLP ENQPLEGTFP CDFAWGVVDN YIQVDTTLSQ FTDLNVYLWD |
| 551 | VHHSKRLIKV DGVVTKKRKS YCVDFAAIQP QIALLQEMHV THFRFSLDWA |
| 601 | LILPLGNQSQ VNHTILQYYR CMASELVRVN ITPVVALWQP MAPNQGLPRL |
| 651 | LARQGAWENP YTALAFAEYA RLCFQELGHH VKLWITMNEP YTRNMTYSAG |
| 701 | HNLLKAHALA WHVYNEKFRH AQNGKISIAL QADWIEPACP FSQKDKEVAE |
| 751 | RVLEFDIGWL AEPIFGSGDY PWVMRDWLNQ RNNFLLPYFT EDEKKLIQGT |
| 801 | FDFLALSHYT TILVDSEKED PIKYNDYLEV QEMTDITWLN SPSQVAWPW |
| 851 | GLRKVLNWLK FKYGDLPMYI ISNGIDDGLH AEDDQLRVYY MQNYINEALK |
| 901 | AHILDGINLC GYFAYSFNDR TAPRFGLYRY AADQFEPKAS MKHYRKIIDS |
| 951 | NGFPGPETLE RFCPEEFTVC TECSFFHTRK SL |

sKlotho with IgG Signal peptide (SEQ ID NO: 45)

| | |
|---|---|
| 1 | MSVLTQVLAL LLLWLTGLGG RRLRAEPGDG AQTWARFSRP PAPEAAGLFQ |
| 51 | GTFPDGFLWA VGSAAYQTEG GWQQHGKGAS IWDTFTHHPL APPGDSRNAS |
| 101 | LPLGAPSPLQ PATGDVASDS YNNVFRDTEA LRELGVTHYR FSISWARVLP |
| 151 | NGSAGVPNRE GLRYYRRLLE RLRELGVQPV VTLYHWDLPQ RLQDAYGGWA |
| 201 | NRALADHFRD YAELCFRHFG GQVKYWITID NPYVVAWHGY ATGRLAPGIR |
| 251 | GSPRLGYLVA HNLLLAHAKV WHLYNTSFRP TQGGQVSIAL SSHWINPRRM |
| 301 | TDHSIKECQK SLDFVLGWFA KPVFIDGDYP ESMKNNLSSI LPDFTESEKK |
| 351 | FIKGTADFFA LCFGPTLSFQ LLDPHMKFRQ LESPNLRQLL SWIDLEFNHP |
| 401 | QIFIVENGWF VSGTTKRDDA KYMYYLKKFI METLKAIKLD GVDVIGYTAW |
| 451 | SLMDGFEWHR GYSIRRGLFY VDFLSQDKML LPKSSALFYQ KLIEKNGFPP |
| 501 | LPENQPLEGT FPCDFAWGVV DNYIQVDTTL SQFTDLNVYL WDVHHSKRLI |
| 551 | KVDGVVTKKR KSYCVDFAAI QPQIALLQEM HVTHFRFSLD WALILPLGNQ |
| 601 | SQVNHTILQY YRCMASELVR VNITPVVALW QPMAPNQGLP RLLARQGAWE |
| 651 | NPYTALAFAE YARLCFQELG HHVKLWITMN EPYTRNMTYS AGHNLLKAHA |
| 701 | LAWHVYNEKF RHAQNGKISI ALQADWIEPA CPFSQKDKEV AERVLEFDIG |
| 751 | WLAEPIFGSG DYPWVMRDWL NQRNNFLLPY FTEDEKKLIQ GTFDFLALSH |
| 801 | YTTILVDSEK EDPIKYNDYL EVQEMTDITW LNSPSQVAW PWGLRKVLNW |
| 851 | LKFKYGDLPM YIISNGIDDG LHAEDDQLRV YYMQNYINEA LKAHILDGIN |
| 901 | LCGYFAYSFN DRTAPRFGLY RYAADQFEPK ASMKHYRKII DSNGFPGPET |
| 951 | LERFCPEEFT VCTECSFFHT RKSL |

What is claimed is:

1.-47. (canceled)

48. A fusion polypeptide comprising a sequence with at least 90% identity to SEQ ID NO:41.

49. The fusion polypeptide of claim 48 further comprising a signal peptide.

50. The fusion polypeptide of claim 49, wherein the signal peptide is a Klotho signal peptide.

51. The fusion polypeptide of claim 49, wherein the signal peptide is an IgG signal peptide.

52. A pharmaceutical composition comprising the fusion polypeptide of claim 48 and a pharmaceutically acceptable carrier.

53. The fusion polypeptide of claim 48, wherein the polypeptide comprises a sequence with at least 95% identity to SEQ ID NO:41.

54. The fusion polypeptide of claim 48, wherein the polypeptide comprises a sequence with at least 96% identity to SEQ ID NO:41.

55. The fusion polypeptide of claim 48, wherein the polypeptide comprises a sequence with at least 97% identity to SEQ ID NO:41.

56. The fusion polypeptide of claim 48, wherein the polypeptide comprises a sequence with at least 98% identity to SEQ ID NO:41.

57. The fusion polypeptide of claim 48, wherein the polypeptide comprises a sequence with at least 99% identity to SEQ ID NO:41.

58. A nucleic acid comprising a sequence that encodes a fusion polypeptide comprising a sequence with at least 90% identity to SEQ ID NO:41.

59. A host cell comprising the nucleic acid of claim 58.

60. A vector comprising the nucleic acid of claim 58.

61. A method for activating Egr-1 in a subject, the method comprising administering to the subject a therapeutically effective amount of a fusion polypeptide comprising a sequence with at least 90% identity to SEQ ID NO:41.

62. The method of claim 61, wherein the subject is in need of a treatment for an age-related condition selected from the group consisting of sarcopenia, skin atrophy, muscle wasting, brain atrophy, atherosclerosis, arteriosclerosis, pulmonary emphysema, osteoporosis, osteoarthritis, immunologic incompetence, high blood pressure, dementia, Huntington's disease, Alzheimer's disease, cataracts, age-related macular degeneration, prostate cancer, stroke, diminished life expectancy, memory loss, wrinkles, impaired kidney function, and age-related hearing loss.

63. The method of claim 61, wherein the subject is in need of a treatment for muscle wasting.

64. The method of claim 61, wherein the subject is in need of a treatment for a metabolic disorder selected from the group consisting of Type II Diabetes, Metabolic Syndrome, hyperglycemia, and obesity.

65. The method of claim 61, wherein the subject is in need of a treatment for hyperphosphatemia or calcinosis.

66. The method of claim 61, wherein the subject is in need of a treatment for chronic renal disease or chronic renal failure.

67. The method of claim 61, wherein the subject is in need of a treatment for cancer.

68. The method of claim 67, wherein the cancer is breast cancer.


SEQUENCE LISTING (53 sequences)

SEQ ID NO: 1 - DNA, 5003 bp, Homo sapiens
cgcgcagcatgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggccgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaacggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactctggaaagattttgtccagaagaattcaccgtgtgtactgagtgcagtttttttcacacccgaaagtctttactggctttcatagcttttctattttttgcttctattatttctctctcccttatattttactactcgaagaaaggcagaagaagttacaaatagttctgaacatttttctattcattcattttgaaataattatgcagacacatcagctgttaaccatttgcacctctaagtgttgtgaaactgtaaatttcatacatttgacttctagaaaacatttttgtggcttatgacagaggttttgaaatgggcataggtgatcgtaaaatattgaataatgcgaatagtgcctgaatttgttctctttttgggtgattaaaaaactgacaggcactataatttctgtaacacactaacaaaagcatgaaaaataggaaccacaccaatgcaacatttgtgcagaaatttgaatgacaagattaggaatattttcttctgcacccacttctaaatttaatgtttttctggaagtagtaattgcaagagttcgaatagaaagttatgtaccaagtaaccatttctcagctgccataataatgcctagtggcttcccctctgtcaaatctagtttcctatggaaaagaagatggcagatacaggagagacgacagagggtcctaggctggaatgttcctttcgaaagcaatgcttctatcaaatactagtattaatttatgtatctggttaatgacatacttggagagcaaattatggaaatgtgtattttatatgatttttgaggtcctgtctaaaccctgtgtccctgagggatctgtctcactggcatcttgttgagggccttgcacataggaaacttttgataagtatctgcggaaaaacaaacatgaatcctgtgatattgggctcttcaggaagcataaagcaattgtgaaatacagtataccgcagtggctctaggtggaggaaaggaggaaaaagtgcttattatgtgcaacattatgattaatctgattatacaccatttttgagcagatcttggaatgaatgacatgacctttccctagagaataaggatgaaataatcactcattctatgaacagtgacactactttctattctttagctgtactgtaatttctttgagttgatagttttacaaattcttaataggttcaaaagcaatctggtctgaataacactggatttgtttctgtgatctctgaggtctattttatgtttttgctgctacttctgtggaagtagctttgaactagttttactttgaactttcacgctgaaacatgctagtgatatctagaaagggctaattaggtctcatcctttaatgccccttaaataagtcttgctgattttcagacagggaagtctctctattacactggagctgttttatagataagtcaatattgtatcaggcaagataaaccaatgtcataacaggcattgccaacctcactgacacagggtcatagtgtataataatatactgtactatataatatatcatctttagaggtatgattttttcatgaaagataagcttttggtaatattcattttaaagtggacttattaaaattggatgctagagaatcaagtttattttatgtatatatttttctgattataagagtaatatatgttcattgtaaaaatttttaaaacacagaaactatatgcaaagaaaaaataaaaattatctataatctcagaacccagaaatagccactattaacatttcctacgtattttattttacatagatcatattgtatatagttagtatctttattaatttttattatgaaactttcctttgtcattattagtcttcaaaagcatgatttttaatagttgttgagtattccaccacaggaatgtatcacaacttaaccgttcccgtttgttagactagtttcttattaatgttgatgaatgttgtttaaaaataattttgttgctacatttactttaatttccttgactgtaaagagaagtaattttgctccttgataaagtattatattaataataaatctgcctgcaactttttgccttctttcataatc

SEQ ID NO: 2 - protein, 1012 aa, Homo sapiens
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLLAFIAFLFFASIISLSLIFYYSKKGRRSYK

SEQ ID NO: 3 - DNA, 3279 bp, Homo sapiens
atcctcagtctcccagttcaagctaatcattgacagagctttacaatcacaagcttttactgaagctttgataagacagtccagcagttggtggcaaatgaagccaggctgtgcggcaggatctccagggaatgaatggattttcttcagcactgatgaaataaccacacgctataggaatacaatgtccaacgggggattgcaaagatctgtcatcctgtcagcacttattctgctacgagctgttactggattctctggagatggaagagctatatggtctaaaaatcctaattttactccggtaaatgaaagtcagctgtttctctatgacactttccctaaaaactttttctggggtattgggactggagcattgcaagtggaagggagttggaagaaggatggaaaaggaccttctatatgggatcatttcatccacacacaccttaaaaatgtcagcagcacgaatggttccagtgacagttatatttttctggaaaaagacttatcagccctggattttataggagtttctttttatcaattttcaatttcctggccaaggcttttccccgatggaatagtaacagttgccaacgcaaaaggtctgcagtactacagtactcttctggacgctctagtgcttagaaacattgaacctatagttactttataccactgggatttgcctttggcactacaagaaaaatatggggggtggaaaaatgataccataatagatatcttcaatgactatgccacatactgtttccagatgtttggggaccgtgtcaaatattggattacaattcacaacccatatctagtggcttggcatgggtatgggacaggtatgcatgcccctggagagaagggaaatttagcagctgtctacactgtgggacacaacttgatcaaggctcactcgaaagtttggcataactacaacacacatttccgcccacatcagaagggttggttatcgatcacgttgggatctcattggatcgagccaaaccggtcggaaaacacgatggatatattcaaatgtcaacaatccatggtttctgtgcttggatggtttgccaaccctatccatggggatggcgactatccagaggggatgagaaagaagttgttctccgttctacccattttctctgaagcagagaagcatgagatgagaggcacagctgatttctttgccttttcttttggacccaacaacttcaagcccctaaacaccatggctaaaatgggacaaaatgtttcacttaatttaagagaagcgctgaactggattaaactggaatacaacaaccctcgaatcttgattgctgagaatggctggttcacagacagtcgtgtgaaaacagaagacaccacggccatctacatgatgaagaatttcctcagccaggtgcttcaagcaataaggttagatgaaatacgagtgtttggttatactgcctggtctctcctggatggctttgaatggcaggatgcttacaccatccgccgaggattattttatgtggattttaacagtaaacagaaagagcggaaacctaagtcttcagcacactactacaaacagatcatacgagaaaatggtttttctttaaaagagtccacgccagatgtgcagggccagtttccctgtgacttctcctggggtgtcactgaatctgttcttaagcccgagtctgtggcttcgtccccacagttcagcgatcctcatctgtacgtgtggaacgccactggcaacagactgttgcaccgagtggaaggggtgaggctgaaaacacgacccgctcaatgcacagattttgtaaacatcaaaaaacaacttgagatgttggcaagaatgaaagtcacccactaccggtttgctctggattgggcctcggtccttcccactggcaacctgtccgcggtgaaccgacaggccctgaggtactacaggtgcgtggtcagtgaggggctgaagcttggcatctccgcgatggtcaccctgtattatccgacccacgcccacctaggcctccccgagcctctgttgcatgccgacgggtggctgaacccatcgacggccgaggccttccaggcctacgctgggctgtgcttccaggagctgggggacctggtgaagctctggatcaccatcaacgagcctaaccggctaagtgacatctacaaccgctctggcaacgacacctacggggcggcgcacaacctgctggtggcccacgccctggcctggcgcctctacgaccggcagttcaggccctcacagcgcggggccgtgtcgctgtcgctgcacgcggactgggcggaacccgccaacccctatgctgactcgcactggagggcggccgagcgcttcctgcagttcgagatcgcctggttcgccgagccgctcttcaagaccggggactaccccgcggccatgagggaatacattgcctccaagcaccgacgggggctttccagctcggccctgccgcgcctcaccgaggccgaaaggaggctgctcaagggcacggtcgacttctgcgcgctcaaccacttcaccactaggttcgtgatgcacgagcagctggccggcagccgctacgactcggacagggacatccagtttctgcaggacatcacccgcctgagctcccccacgcgcctggctgtgattccctggggggtgcgcaagctgctgcggtgggtccggaggaactacggcgacatggacatttacatcaccgccagtggcatcgacgaccaggctctggaggatgaccggctccggaagtactacctagggaagtaccttcaggaggtgctgaaagcatacctgattgataaagtcagaatcaaaggctattatgcattcaaactggctgaagagaaatctaaacccagatttggattcttcacatctgattttaaagctaaatcctcaatacaattttacaacaaagtgatcagcagcaggggcttcccttttgagaacagtagttctagatgcagtcagacccaagaaaatacagagtgcactgtctgcttattccttgtgcagaagaaaccactgatattcctgggttgttgcttcttctccaccctggttctactcttatcaattgccatttttcaaaggcagaagagaagaaagttttggaaagcaaaaaacttacaacacataccattaaagaaaggcaagagagttgttagctaaactgatctgtctgcatgatagacagtttaaaaattcatcccagttcc

SEQ ID NO: 4 - protein, 1044 aa, Homo sapiens
MKPGCAAGSPGNEWIFFSTDEITTRYRNTMSNGGLQRSVILSALILLRAVTGFSGDGRAIWSKNPNFTPVNESQLFLYDTFPKNFFWGIGTGALQVEGSWKKDGKGPSIWDHFIHTHLKNVSSTNGSSDSYIFLEKDLSALDFIGVSFYQFSISWPRLFPDGIVTVANAKGLQYYSTLLDALVLRNIEPIVTLYHWDLPLALQEKYGGWKNDTIIDIFNDYATYCFQMFGDRVKYWITIHNPYLVAWHGYGTGMHAPGEKGNLAAVYTVGHNLIKAHSKVWHNYNTHFRPHQKGWLSITLGSHWIEPNRSENTMDIFKCQQSMVSVLGWFANPIHGDGDYPEGMRKKLFSVLPIFSEAEKHEMRGTADFFAFSFGPNNFKPLNTMAKMGQNVSLNLREALNWIKLEYNNPRILIAENGWFTDSRVKTEDTTAIYMMKNFLSQVLQAIRLDEIRVFGYTAWSLLDGFEWQDAYTIRRGLFYVDFNSKQKERKPKSSAHYYKQIIRENGFSLKESTPDVQGQFPCDFSWGVTESVLKPESVASSPQFSDPHLYVWNATGNRLLHRVEGVRLKTRPAQCTDFVNIKKQLEMLARMKVTHYRFALDWASVLPTGNLSAVNRQALRYYRCVVSEGLKLGISAMVTLYYPTHAHLGLPEPLLHADGWLNPSTAEAFQAYAGLCFQELGDLVKLWITINEPNRLSDIYNRSGNDTYGAAHNLLVAHALAWRLYDRQFRPSQRGAVSLSLHADWAEPANPYADSHWRAAERFLQFEIAWFAEPLFKTGDYPAAMREYIASKHRRGLSSSALPRLTEAERRLLKGTVDFCALNHFTTRFVMHEQLAGSRYDSDRDIQFLQDITRLSSPTRLAVIPWGVRKLLRWVRRNYGDMDIYITASGIDDQALEDDRLRKYYLGKYLQEVLKAYLIDKVRIKGYYAFKLAEEKSKPRFGFFTSDFKAKSSIQFYNKVISSRGFPFENSSSRCSQTQENTECTVCLFLVQKKPLIFLGCCFFSTLVLLLSIAIFQRQKRRKFWKAKNLQHIPLKKGKRVVS

SEQ ID NO: 5 - protein, 449 aa, Homo sapiens
QGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGF

SEQ ID NO: 6 - protein, 437 aa, Homo sapiens
GTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGF

SEQ ID NO: 7 - protein, 949 aa, Homo sapiens
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSL

SEQ ID NO: 8 - protein, 33 aa, Homo sapiens
MPASAPPRRPRPPPPSLSLLLVLLGLGGRRLRA

SEQ ID NO: 9 - protein, 25 aa, Homo sapiens
MSVLTQVLALLLLWLTGTRCRRLRA

SEQ ID NO: 10 - DNA, 45 bp, Artificial
  linker
ggaggtggaggttcaggaggtggaggttcaggaggtggaggttca

SEQ ID NO: 11 - protein, 15 aa, Artificial
  linker
GGGGSGGGGSGGGGS

SEQ ID NO: 12 - protein, 5 aa, Artificial
  linker
GGGGS

SEQ ID NO: 13 - protein, 1 aa, Artificial
  linker
G

SEQ ID NO: 14 - protein, 2 aa, Artificial
  linker
GG

SEQ ID NO: 15 - protein, 2 aa, Artificial
  linker
GS

SEQ ID NO: 16 - protein, 3 aa, Artificial
  linker
GGS

SEQ ID NO: 17 - protein, 1 aa, Artificial
  linker
A

SEQ ID NO: 18 - protein, 2 aa, Artificial
  linker
AA

SEQ ID NO: 19 - protein, 1228 aa, Artificial
  fusion polypeptide
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLGSGGGGSGGGGSGGGGSLKYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFI

SEQ ID NO: 20 - protein, 1220 aa, Artificial
  fusion polypeptide
MSVLTQVLALLLLWLTGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLGSGGGGSGGGGSGGGGSLKYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFI

SEQ ID NO: 21 - protein, 762 aa, Artificial
  fusion polypeptide
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGSGGGGSGGGGSGGGGSLKYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFI

SEQ ID NO: 22 - protein, 752 aa, Artificial
  fusion polypeptide
MPASAPPRRPRPPPPSLSLLLVLLGLGGRRLPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLGSGGGGSGGGGSGGGGSLKYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFI

SEQ ID NO: 23 - protein, 1215 aa, Artificial
  fusion polypeptide
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGSGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPEFGSGGGGSGGGGSGGGGSLKYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFI

SEQ ID NO: 24 - protein, 1189 aa, Artificial
  fusion polypeptide
MPASAPPRRPRPPPPSLSLLLVLLGLGGRRLPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFGSGGGGSGGGGSGGGGSLKYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFI

SEQ ID NO: 25 - protein, 1219 aa, Artificial [sequence omitted]

SEQ ID NO: 26 - protein, 700 aa, Artificial
  fusion polypeptide
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFIQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGF

SEQ ID NO: 27 - protein, 688 aa, Artificial
  fusion polypeptide
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFIGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGF

SEQ ID NO: 28 - protein, 1149 aa, Artificial
  fusion polypeptide
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFIQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGF

SEQ ID NO: 29 - protein, 1125 aa, Artificial
  fusion polypeptide
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFIGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGF

SEQ ID NO: 30 - DNA, 2157 bp, Homo sapiens
gctcccagccaagaacctcggggccgctgcgcggtggggaggagttccccgaaacccggccgctaagcgaggcctcctcctcccgcagatccgaacggcctgggcggggtcaccccggctgggacaagaagccgccgcctgcctgcccgggcccggggagggggctggggctggggccggaggcggggtgtgagtgggtgtgtgcggggggcggaggcttgatgcaatcccgataagaaatgctcgggtgtcttgggcacctacccgtggggcccgtaaggcgctactatataaggctgccggcccggagccgccgcgccgtcagagcaggagcgctgcgtccaggatctagggccacgaccatcccaacccggcactcacagccccgcagcgcatcccggtcgccgcccagcctcccgcacccccatcgccggagctgcgccgagagccccagggaggtgccatgcggagcgggtgtgtggtggtccacgtatggatcctggccggcctctggctggccgtggccgggcgccccctcgccttctcggacgcggggccccacgtgcactacggctggggcgaccccatccgcctgcggcacctgtacacctccggcccccacgggctctccagctgcttcctgcgcatccgtgccgacggcgtcgtggactgcgcgcggggccagagcgcgcacagtttgctggagatcaaggcagtcgctctgcggaccgtggccatcaagggcgtgcacagcgtgcggtacctctgcatgggcgccgacggcaagatgcaggggctgcttcagtactcggaggaagactgtgctttcgaggaggagatccgcccagatggctacaatgtgtaccgatccgagaagcaccgcctcccggtctccctgagcagtgccaaacagcggcagctgtacaagaacagaggctttcttccactctctcatttcctgcccatgctgcccatggtcccagaggagcctgaggacctcaggggccacttggaatctgacatgttctcttcgcccctggagaccgacagcatggacccatttgggcttgtcaccggactggaggccgtgaggagtcccagctttgagaagtaactgagaccatgcccgggcctcttcactgctgccaggggctgtggtacctgcagcgtgggggacgtgcttctacaagaacagtcctgagtccacgttctgtttagctttaggaagaaacatctagaagttgtacatattcagagttttccattggcagtgccagtttctagccaatagacttgtctgatcataacattgtaagcctgtagcttgcccagctgctgcctgggcccccattctgctccctcgaggttgctggacaagctgctgcactgtctcagttctgcttgaatacctccatcgatggggaactcacttcctttggaaaaattcttatgtcaagctgaaattctctaattttttctcatcacttccccaggagcagccagaagacaggcagtagttttaatttcaggaacaggtgatccactctgtaaaacagcaggtaaatttcactcaaccccatgtgggaattgatctatatctctacttccagggaccatttgcccttcccaaatccctccaggccagaactgactggagcaggcatggcccaccaggcttcaggagtaggggaagcctggagccccactccagccctgggacaacttgagaattccccctgaggccagttctgtcatggatgctgtcctgagaataacttgctgtcccggtgtcacctgcttccatctcccagcccaccagccctctgcccacctcacatgcctccccatggattggggcctcccaggccccccaccttatgtcaacctgcacttcttgttcaaaaatcaggaaaagaaaagatttgaagaccccaagtcttgtcaataacttgctgtgtggaagcagcgggggaagacctagaaccctttccccagcacttggttttccaacatgatatttatgagtaatttattttgatatgtacatctcttattttcttacattatttatgcccccaaattatatttatgtatgtaagtgaggtttgttttgtatattaaaatggagtttgtttgtaaaaaaaaaaaaaaaaaaaa

SEQ ID NO: 31 - protein, 216 aa, Homo sapiens
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 32 - DNA, 940 bp, Homo sapiens
ctgtcagctgaggatccagccgaaagaggagccaggcactcaggccacctgagtctactcacctggacaactggaatctggcaccaattctaaaccactcagcttctccgagctcacaccccggagatcacctgaggacccgagccattgatggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctgaagccagaggctgtttactatgacatctcctctttatttattaggttatttatcttatttatttttttatttttcttacttgagataataaagagttccagaggagaaaaaaaaaaaaaaaaaaaaaaaaaaaaaaaaaaaaaaaaaaaaaaaaaaaaaaa

SEQ ID NO: 33 - protein, 209 aa, Homo sapiens
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 34 - DNA, 3018 bp, Homo sapiens
cggcaaaaaggagggaatccagtctaggatcctcacaccagctacttgcaagggagaaggaaaaggccagtaaggcctgggccaggagagtcccgacaggagtgtcaggtttcaatctcagcaccagccactcagagcagggcacgatgttgggggcccgcctcaggctctgggtctgtgccttgtgcagcgtctgcagcatgagcgtcctcagagcctatcccaatgcctccccactgctcggctccagctggggtggcctgatccacctgtacacagccacagccaggaacagctaccacctgcagatccacaagaatggccatgtggatggcgcaccccatcagaccatctacagtgccctgatgatcagatcagaggatgctggctttgtggtgattacaggtgtgatgagcagaagatacctctgcatggatttcagaggcaacatttttggatcacactatttcgacccggagaactgcaggttccaacaccagacgctggaaaacgggtacgacgtctaccactctcctcagtatcacttcctggtcagtctgggccgggcgaagagagccttcctgccaggcatgaacccacccccgtactcccagttcctgtcccggaggaacgagatccccctaattcacttcaacacccccataccacggcggcacacccggagcgccgaggacgactcggagcgggaccccctgaacgtgctgaagccccgggcccggatgaccccggccccggcctcctgttcacaggagctcccgagcgccgaggacaacagcccgatggccagtgacccattaggggtggtcaggggcggtcgagtgaacacgcacgctgggggaacgggcccggaaggctgccgccccttcgccaagttcatctagggtcgctggaagggcaccctctttaacccatccctcagcaaacgcagctcttcccaaggaccaggtcccttgacgttccgaggatgggaaaggtgacaggggcatgtatggaatttgctgcttctctggggtcccttccacaggaggtcctgtgagaaccaacctttgaggcccaagtcatggggtttcaccgccttcctcactccatatagaacacctttcccaataggaaaccccaacaggtaaactagaaatttccccttcatgaaggtagagagaaggggtctctcccaacatatttctcttccttgtgcctctcctctttatcacttttaagcataaaaaaaaaaaaaaaaaaaaaaaaaaaaaaaagcagtgggttcctgagctcaagactttgaaggtgtagggaagaggaaatcggagatcccagaagcttctccactgccctatgcatttatgttagatgccccgatcccactggcatttgagtgtgcaaaccttgacattaacagctgaatggggcaagttgatgaaaacactactttcaagccttcgttcttccttgagcatctctggggaagagctgtcaaaagactggtggtaggctggtgaaaacttgacagctagacttgatgcttgctgaaatgaggcaggaatcataatagaaaactcagcctccctacagggtgagcaccttctgtctcgctgtctccctctgtgcagccacagccagagggcccagaatggccccactctgttcccaagcagttcatgatacagcctcaccttttggccccatctctggtttttgaaaatttggtctaaggaataaatagcttttacactggctcacgaaaatctgccctgctagaatttgcttttcaaaatggaaataaattccaactctcctaagaggcatttaattaaggctctacttccaggttgagtaggaatccattctgaacaaactacaaaaatgtgactgggaagggggctttgagagactgggactgctctgggttaggttttctgtggactgaaaaatcgtgtccttttctctaaatgaagtggcatcaaggactcagggggaaagaaatcaggggacatgttatagaagttatgaaaagacaaccacatggtcaggctcttgtctgtggtctctagggctctgcagcagcagtggctcttcgattagttaaaactctcctaggctgacacatctgggtctcaatccccttggaaattcttggtgcattaaatgaagccttaccccattactgcggttcttcctgtaagggggctccattttcctccctctctttaaatgaccacctaaaggacagtatattaacaagcaaagtcgattcaacaacagcttcttcccagtcacttttttttttctcactgccatcacatactaaccttatactttgatctattctttttggttatgagagaaatgttgggcaactgtttttacctgatggttttaagctgaacttgaaggactggttcctattctgaaacagtaaaactatgtataatagtatatagccatgcatggcaaatattttaatatttctgttttcatttcctgttggaaatattatcctgcataatagctattggaggctcctcagtgaaagatcccaaaaggattttggtggaaaactagttgtaatctcacaaactcaacactaccatcaggggttttctttatggcaaagccaaaatagctcctacaatttcttatatccctcgtcatgtggcagtatttatttatttatttggaagtttgcctatccttctatatttatagatatttataaaaatgtaacccctttttcctttcttctgtttaaaataaaaataaaatttatctcagcttctgttagcttatcctctttgtagtactacttaaaagcatgtcggaatataagaataaaaaggattatgggaggggaacattagggaaatccagagaaggcaaaattgaaaaaaagattttagaattttaaaattttcaaagatttcttccattcataaggagactcaatgattttaattgatctagacagaattatttaagttttatcaatattggatttctggt

SEQ ID NO: 35 - protein, 251 aa, Homo sapiens
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTRSAEDDSERDPLNVLKPRARMTPAPASCSQELPSAEDNSPMASDPLGVVRGGRVNTHAGGTGPEGCRPFAKFI

SEQ ID NO: 36 - protein, 251 aa, Artificial
  fusion polypeptide
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFI

SEQ ID NO: 37 - protein, 431 aa, Homo sapiens
YDTFPKNFFWGIGTGALQVEGSWKKDGKGPSIWDHFIHTHLKNVSSTNGSSDSYIFLEKDLSALDFIGVSFYQFSISWPRLFPDGIVTVANAKGLQYYSTLLDALVLRNIEPIVTLYHWDLPLALQEKYGGWKNDTIIDIFNDYATYCFQMFGDRVKYWITIHNPYLVAWHGYGTGMHAPGEKGNLAAVYTVGHNLIKAHSKVWHNYNTHFRPHQKGWLSITLGSHWIEPNRSENTMDIFKCQQSMVSVLGWFANPIHGDGDYPEGMRKKLFSVLPIFSEAEKHEMRGTADFFAFSFGPNNFKPLNTMAKMGQNVSLNLREALNWIKLEYNNPRILIAENGWFTDSRVKTEDTTAIYMMKNFLSQVLQAIRLDEIRVFGYTAWSLLDGFEWQDAYTIRRGLFYVDFNSKQKERKPKSSAHYYKQIIRENGF

SEQ ID NO: 38 - protein, 397 aa, Homo sapiens
TRPAQCTDFVNIKKQLEMLARMKVTHYRFALDWASVLPTGNLSAVNRQALRYYRCVVSEGLKLGISAMVTLYYPTHAHLGLPEPLLHADGWLNPSTAEAFQAYAGLCFQELGDLVKLWITINEPNRLSDIYNRSGNDTYGAAHNLLVAHALAWRLYDRQFRPSQRGAVSLSLHADWAEPANPYADSHWRAAERFLQFEIAWFAEPLFKTGDYPAAMREYIASKHRRGLSSSALPRLTEAERRLLKGTVDFCALNHFTTRFVMHEQLAGSRYDSDRDIQFLQDITRLSSPTRLAVIPWGVRKLLRWVRRNYGDMDIYITASGIDDQALEDDRLRKYYLGKYLQEVLKAYLIDKVRIKGYYAFKLAEEKSKPRFGFFTSDFKAKSSIQFYNKVISSRGF

SEQ ID NO: 39 - protein, 946 aa, Homo sapiens
GFSGDGRAIWSKNPNFTPVNESQLFLYDTFPKNFFWGIGTGALQVEGSWKKDGKGPSIWDHFIHTHLKNVSSTNGSSDSYIFLEKDLSALDFIGVSFYQFSISWPRLFPDGIVTVANAKGLQYYSTLLDALVLRNIEPIVTLYHWDLPLALQEKYGGWKNDTIIDIFNDYATYCFQMFGDRVKYWITIHNPYLVAWHGYGTGMHAPGEKGNLAAVYTVGHNLIKAHSKVWHNYNTHFRPHQKGWLSITLGSHWIEPNRSENTMDIFKCQQSMVSVLGWFANPIHGDGDYPEGMRKKLFSVLPIFSEAEKHEMRGTADFFAFSFGPNNFKPLNTMAKMGQNVSLNLREALNWIKLEYNNPRILIAENGWFTDSRVKTEDTTAIYMMKNFLSQVLQAIRLDEIRVFGYTAWSLLDGFEWQDAYTIRRGLFYVDFNSKQKERKPKSSAHYYKQIIRENGFSLKESTPDVQGQFPCDFSWGVTESVLKPESVASSPQFSDPHLYVWNATGNRLLHRVEGVRLKTRPAQCTDFVNIKKQLEMLARMKVTHYRFALDWASVLPTGNLSAVNRQALRYYRCVVSEGLKLGISAMVTLYYPTHAHLGLPEPLLHADGWLNPSTAEAFQAYAGLCFQELGDLVKLWITINEPNRLSDIYNRSGNDTYGAAHNLLVAHALAWRLYDRQFRPSQRGAVSLSLHADWAEPANPYADSHWRAAERFLQFEIAWFAEPLFKTGDYPAAMREYIASKHRRGLSSSALPRLTEAERRLLKGTVDFCALNHFTTRFVMHEQLAGSRYDSDRDIQFLQDITRLSSPTRLAVIPWGVRKLLRWVRRNYGDMDIYITASGIDDQALEDDRLRKYYLGKYLQEVLKAYLIDKVRIKGYYAFKLAEEKSKPRFGFFTSDFKAKSSIQFYNKVISSRGFPFENSSSRCSQTQENTECTVCLFLVQKKPL

SEQ ID NO: 40 - protein, 1195 aa, Artificial
  fusion polypeptide
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLGSGGGGSGGGGSGGGGSLKYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTRSAEDDSERDPLNVLKPRARMTPAPASCSQELPSAEDNSPMASDPLGVVRGGRVNTHAGGTGPEGCRPFAKFI

SEQ ID NO: 41 - protein, 1195 aa, Artificial
  fusion polypeptide
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLGSGGGGSGGGGSGGGGSLKYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFI

SEQ ID NO: 42 - protein, 227 aa, Artificial
  fusion polypeptide
YPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTRSAEDDSERDPLNVLKPRARMTPAPASCSQELPSAEDNSPMASDPLGVVRGGRVNTHAGGTGPEGCRPFAKFI

SEQ ID NO: 43 - protein, 227 aa, Artificial
  fusion polypeptide
YPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFI

SEQ ID NO: 44 - protein, 982 aa, Artificial
  fusion polypeptide
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSL

SEQ ID NO: 45 - protein, 974 aa, Artificial
  fusion polypeptide
MSVLTQVLALLLLWLTGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSL

SEQ ID NO: 46 - protein, 4380 aa, Artificial
  fusion peptide derived from human
ATGCCCGCCAGCGCCCCGCCGCGCCGCCCGCGGCCGCCGCCGCCGTCGCTGTCGCTGCTGCTGGTGCTGCTGGGCCTGGGCGGCCGCCGCCTGCGTGCGGAGCCGGGCGACGGCGCGCAGACCTGGGCCCGTTTCTCGCGGCCTCCTGCCCCCGAGGCCGCGGGCCTCTTCCAGGGCACCTTCCCCGACGGCTTCCTCTGGGCCGTGGGCAGCGCCGCCTACCAGACCGAGGGCGGCTGGCAGCAGCACGGCAAGGGTGCGTCCATCTGGGATACGTTCACCCACCACCCCCTGGCACCCCCGGGAGACTCCCGGAACGCCAGTCTGCCGTTGGGCGCCCCGTCGCCGCTGCAGCCCGCCACCGGGGACGTAGCCAGCGACAGCTACAACAACGTCTTCCGCGACACGGAGGCGCTGCGCGAGCTCGGGGTCACTCACTACCGCTTCTCCATCTCGTGGGCGCGAGTGCTCCCCAATGGCAGCGCGGGCGTCCCCAACCGCGAGGGGCTGCGCTACTACCGGCGCCTGCTGGAGCGGCTGCGGGAGCTGGGCGTGCAGCCCGTGGTCACCCTGTACCACTGGGACCTGCCCCAGCGCCTGCAGGACGCCTACGGCGGCTGGGCCAACCGCGCCCTGGCCGACCACTTCAGGGATTACGCGGAGCTCTGCTTCCGCCACTTCGGCGGTCAGGTCAAGTACTGGATCACCATCGACAACCCCTACGTGGTGGCCTGGCACGGCTACGCCACCGGGCGCCTGGCCCCCGGCATCCGGGGCAGCCCGCGGCTCGGGTACCTGGTGGCGCACAACCTCCTCCTGGCTCATGCCAAAGTCTGGCATCTCTACAATACTTCTTTCCGTCCCACTCAGGGAGGTCAGGTGTCCATTGCCCTAAGCTCTCACTGGATCAATCCTCGAAGAATGACCGACCACAGCATCAAAGAATGTCAAAAATCTCTGGACTTTGTACTAGGTTGGTTTGCCAAACCCGTATTTATTGATGGTGACTATCCCGAGAGCATGAAGAATAACCTTTCATCTATTCTGCCTGATTTTACTGAATCTGAGAAAAAGTTCATCAAAGGAACTGCTGACTTTTTTGCTCTTTGCTTTGGACCCACCTTGAGTTTTCAACTTTTGGACCCTCACATGAAGTTCCGCCAATTGGAATCTCCCAACCTGAGGCAACTGCTTTCCTGGATTGACCTTGAATTTAACCATCCTCAAATATTTATTGTGGAAAATGGCTGGTTTGTCTCAGGGACCACCAAGAGAGATGATGCCAAATATATGTATTACCTCAAAAAGTTCATCATGGAAACCTTAAAAGCCATCAAGCTGGATGGGGTGGATGTCATCGGGTATACCGCATGGTCCCTCATGGATGGTTTCGAGTGGCACAGAGGTTACAGCATCAGGCGTGGACTCTTCTATGTTGACTTTCTAAGCCAGGACAAGATGTTGTTGCCAAAGTCTTCAGCCTTGTTCTACCAAAAGCTGATAGAGAAAAATGGCTTCCCTCCTTTACCTGAAAATCAGCCCCTAGAAGGGACATTTCCCTGTGACTTTGCTTGGGGAGTTGTTGACAACTACATTCAAGTAGATACCACTCTGTCTCAGTTTACCGACCTGAATGTTTACCTGTGGGATGTCCACCACAGTAAAAGGCTTATTAAAGTGGATGGGGTTGTGACCAAGAAGAGGAAATCCTACTGTGTTGACTTTGCTGCCATCCAGCCCCAGATCGCTTTACTCCAGGAAATGCACGTTACACATTTTCGCTTCTCCCTGGACTGGGCCCTGATTCTCCCTCTGGGTAACCAGTCCCAGGTGAACCACACCATCCTGCAGTACTATCGCTGCATGGCCAGCGAGCTTGTCCGTGTCAACATCACCCCAGTGGTGGCCCTGTGGCAGCCTATGGCCCCGAACCAAGGACTGCCGCGCCTCCTGGCCAGGCAGGGCGCCTGGGAGAACCCCTACACTGCCCTGGCCTTTGCAGAGTATGCCCGACTGTGCTTTCAAGAGCTCGGCCATCACGTCAAGCTTTGGATAACGATGAATGAGCCGTATACAAGGAATATGACATACAGTGCTGGCCACAACCTTCTGAAGGCCCATGCCCTGGCTTGGCATGTGTACAATGAAAAGTTTAGGCATGCTCAGAATGGGAAAATATCCATAGCCTTGCAGGCTGATTGGATAGAACCTGCCTGCCCTTTCTCCCAAAAGGACAAAGAGGTGGCCGAGAGAGTTTTGGAATTTGACATTGGCTGGCTGGCTGAGCCCATTTTCGGCTCTGGAGATTATCCATGGGTGATGAGGGACTGGCTGAACCAAAGAAACAATTTTCTTCTTCCTTATTTCACTGAAGATGAAAAAAAGCTAATCCAGGGTACCTTTGACTTTTTGGCTTTAAGCCATTATACCACCATCCTTGTAGACTCAGAAAAAGAAGATCCAATAAAATACAATGATTACCTAGAAGTGCAAGAAATGACCGACATCACGTGGCTCAACTCCCCCAGTCAGGTGGCGGTAGTGCCCTGGGGGTTGCGCAAAGTGCTGAACTGGCTGAAGTTCAAGTACGGAGACCTCCCCATGTACATAATATCCAACGGAATCGATGACGGGCTGCATGCTGAGGACGACCAGCTGAGGGTGTATTATATGCAGAATTACATAAACGAAGCTCTCAAAGCCCACATACTGGATGGTATCAATCTTTGCGGATACTTTGCTTATTCGTTTAACGACCGCACAGCTCCGAGGTTTGGCCTCTATCGTTATGCTGCAGATCAGTTTGAGCCCAAGGCATCCATGAAACATTACAGGAAAATTATTGACAGCAATGGTTTCCCGGGCCCAGAAACTCTGGAAAGATTTTGTCCAGAAGAATTCACCGTGTGTACTGAGTGCAGTTTTTTTCACACCCGAAAGTCTTTAGGATCCGGAGGTGGAGGTTCAGGAGGTGGAGGTTCAGGAGGTGGAGGTTCACTTAAGTATCCCAATGCCTCCCCACTGCTCGGCTCCAGCTGGGGTGGCCTGATCCACCTGTACACAGCCACAGCCAGGAACAGCTACCACCTGCAGATCCACAAGAATGGCCATGTGGATGGCGCACCCCATCAGACCATCTACAGTGCCCTGATGATCAGATCAGAGGATGCTGGCTTTGTGGTGATTACAGGTGTGATGAGCAGAAGATACCTCTGCATGGATTTCAGAGGCAACATTTTTGGATCACACTATTTCGACCCGGAGAACTGCAGGTTCCAACACCAGACGCTGGAAAACGGGTACGACGTCTACCACTCTCCTCAGTATCACTTCCTGGTCAGTCTGGGCCGGGCGAAGAGAGCCTTCCTGCCAGGCATGAACCCACCCCCGTACTCCCAGTTCCTGTCCCGGAGGAACGAGATCCCCCTAATTCACTTCAACACCCCCATACCACGGCGGCACACCCAGAGCGCCGAGGACGACTCGGAGCGGGACCCCCTGAACGTGCTGAAGCCCCGGGCCCGGATGACCCCGGCCCCGGCCTCCTGTTCACAGGAGCTCCCGAGCGCCGAGGACAACAGCCCGATGGCCAGTGACCCATTAGGGGTGGTCAGGGGCGGTCGAGTGAACACGCACGCTGGGGGAACGGGCCCGGAAGGCTGCCGCCCCTTCGCCAAGTTCATCGGAGGTGGAGGTTCAAAAACCCACACGTGTCCTCCTTGTCCTGCCCCAGAAGCAGCAGGTGGTCCATCAGTTTTTCTTTTCCCTCCCAAACCCAAGGATACGCTGATGATCTCTCGCACGCCTGAGGTGACATGCGTCGTAGTAGACGTGAGCCACGAAGATCCCGAGGTGAAGTTCAATTGGTATGTGGACGGAGTAGAAGTGCATAACGCGAAAACTAAGCCGCGCGAGGAACAATATAACAGTACTTACAGGGTGGTATCCGTGCTCACAGTCCTGCACCAGGACTGGCTGAACGGTAAGGAATACAAGTGCAAAGTAAGCAACAAGGCACTTCCCGCGCCTATTGAGAAAACAATCTCCAAGGCGAAGGGACAACCAAGAGAACCTCAGGTTTACACTCTCCCGCCTTCCAGGGAAGAGATGACCAAAAATCAAGTTTCCCTGACTTGCCTCGTCAAAGGATTCTACCCTTCCGACATTGCTGTTGAATGGGAAAGCAATGGACAACCAGAGAACAACTACAAGACAACACCCCCGGTGCTGGATAGTGACGGATCTTTCTTTCTCTACTCAAAGCTGACCGTGGATAAGTCCAGGTGGCAGCAGGGAAACGTGTTTTCCTGCTCTGTCATGCATGAAGCGCTGCATAATCACTATACCCAGAAGTCTCTGAGCTTGAGCCCAGGCAAGTAA

SEQ ID NO: 47 - protein, 1459 aa, Artificial [sequence omitted]

SEQ ID NO: 48 - protein, 4353 aa, Artificial
  fusion peptide derived from human
ATGCCCGCCAGCGCCCCGCCGCGCCGCCCGCGGCCGCCGCCGCCGTCGCTGTCGCTGCTGCTGGTGCTGCTGGGCCTGGGCGGCCGCCGCCTGCGTGCGGAGCCGGGCGACGGCGCGCAGACCTGGGCCCGTTTCTCGCGGCCTCCTGCCCCCGAGGCCGCGGGCCTCTTCCAGGGCACCTTCCCCGACGGCTTCCTCTGGGCCGTGGGCAGCGCCGCCTACCAGACCGAGGGCGGCTGGCAGCAGCACGGCAAGGGTGCGTCCATCTGGGATACGTTCACCCACCACCCCCTGGCACCCCCGGGAGACTCCCGGAACGCCAGTCTGCCGTTGGGCGCCCCGTCGCCGCTGCAGCCCGCCACCGGGGACGTAGCCAGCGACAGCTACAACAACGTCTTCCGCGACACGGAGGCGCTGCGCGAGCTCGGGGTCACTCACTACCGCTTCTCCATCTCGTGGGCGCGAGTGCTCCCCAATGGCAGCGCGGGCGTCCCCAACCGCGAGGGGCTGCGCTACTACCGGCGCCTGCTGGAGCGGCTGCGGGAGCTGGGCGTGCAGCCCGTGGTCACCCTGTACCACTGGGACCTGCCCCAGCGCCTGCAGGACGCCTACGGCGGCTGGGCCAACCGCGCCCTGGCCGACCACTTCAGGGATTACGCGGAGCTCTGCTTCCGCCACTTCGGCGGTCAGGTCAAGTACTGGATCACCATCGACAACCCCTACGTGGTGGCCTGGCACGGCTACGCCACCGGGCGCCTGGCCCCCGGCATCCGGGGCAGCCCGCGGCTCGGGTACCTGGTGGCGCACAACCTCCTCCTGGCTCATGCCAAAGTCTGGCATCTCTACAATACTTCTTTCCGTCCCACTCAGGGAGGTCAGGTGTCCATTGCCCTAAGCTCTCACTGGATCAATCCTCGAAGAATGACCGACCACAGCATCAAAGAATGTCAAAAATCTCTGGACTTTGTACTAGGTTGGTTTGCCAAACCCGTATTTATTGATGGTGACTATCCCGAGAGCATGAAGAATAACCTTTCATCTATTCTGCCTGATTTTACTGAATCTGAGAAAAAGTTCATCAAAGGAACTGCTGACTTTTTTGCTCTTTGCTTTGGACCCACCTTGAGTTTTCAACTTTTGGACCCTCACATGAAGTTCCGCCAATTGGAATCTCCCAACCTGAGGCAACTGCTTTCCTGGATTGACCTTGAATTTAACCATCCTCAAATATTTATTGTGGAAAATGGCTGGTTTGTCTCAGGGACCACCAAGAGAGATGATGCCAAATATATGTATTACCTCAAAAAGTTCATCATGGAAACCTTAAAAGCCATCAAGCTGGATGGGGTGGATGTCATCGGGTATACCGCATGGTCCCTCATGGATGGTTTCGAGTGGCACAGAGGTTACAGCATCAGGCGTGGACTCTTCTATGTTGACTTTCTAAGCCAGGACAAGATGTTGTTGCCAAAGTCTTCAGCCTTGTTCTACCAAAAGCTGATAGAGAAAAATGGCTTCCCTCCTTTACCTGAAAATCAGCCCCTAGAAGGGACATTTCCCTGTGACTTTGCTTGGGGAGTTGTTGACAACTACATTCAAGTAGATACCACTCTGTCTCAGTTTACCGACCTGAATGTTTACCTGTGGGATGTCCACCACAGTAAAAGGCTTATTAAAGTGGATGGGGTTGTGACCAAGAAGAGGAAATCCTACTGTGTTGACTTTGCTGCCATCCAGCCCCAGATCGCTTTACTCCAGGAAATGCACGTTACACATTTTCGCTTCTCCCTGGACTGGGCCCTGATTCTCCCTCTGGGTAACCAGTCCCAGGTGAACCACACCATCCTGCAGTACTATCGCTGCATGGCCAGCGAGCTTGTCCGTGTCAACATCACCCCAGTGGTGGCCCTGTGGCAGCCTATGGCCCCGAACCAAGGACTGCCGCGCCTCCTGGCCAGGCAGGGCGCCTGGGAGAACCCCTACACTGCCCTGGCCTTTGCAGAGTATGCCCGACTGTGCTTTCAAGAGCTCGGCCATCACGTCAAGCTTTGGATAACGATGAATGAGCCGTATACAAGGAATATGACATACAGTGCTGGCCACAACCTTCTGAAGGCCCATGCCCTGGCTTGGCATGTGTACAATGAAAAGTTTAGGCATGCTCAGAATGGGAAAATATCCATAGCCTTGCAGGCTGATTGGATAGAACCTGCCTGCCCTTTCTCCCAAAAGGACAAAGAGGTGGCCGAGAGAGTTTTGGAATTTGACATTGGCTGGCTGGCTGAGCCCATTTTCGGCTCTGGAGATTATCCATGGGTGATGAGGGACTGGCTGAACCAAAGAAACAATTTTCTTCTTCCTTATTTCACTGAAGATGAAAAAAAGCTAATCCAGGGTACCTTTGACTTTTTGGCTTTAAGCCATTATACCACCATCCTTGTAGACTCAGAAAAAGAAGATCCAATAAAATACAATGATTACCTAGAAGTGCAAGAAATGACCGACATCACGTGGCTCAACTCCCCCAGTCAGGTGGCGGTAGTGCCCTGGGGGTTGCGCAAAGTGCTGAACTGGCTGAAGTTCAAGTACGGAGACCTCCCCATGTACATAATATCCAACGGAATCGATGACGGGCTGCATGCTGAGGACGACCAGCTGAGGGTGTATTATATGCAGAATTACATAAACGAAGCTCTCAAAGCCCACATACTGGATGGTATCAATCTTTGCGGATACTTTGCTTATTCGTTTAACGACCGCACAGCTCCGAGGTTTGGCCTCTATCGTTATGCTGCAGATCAGTTTGAGCCCAAGGCATCCATGAAACATTACAGGAAAATTATTGACAGCAATGGTTTCCCGGGCCCAGAAACTCTGGAAAGATTTTGTCCAGAAGAATTCACCGTGTGTACTGAGTGCAGTTTTTTTCACACCCGAAAGTCTTTAGGATCCGGAGGTGGAGGTTCAGGAGGTGGAGGTTCAGGAGGTGGAGGTTCACTTAAGTATCCCAATGCCTCCCCACTGCTCGGCTCCAGCTGGGGTGGCCTGATCCACCTGTACACAGCCACAGCCAGGAACAGCTACCACCTGCAGATCCACAAGAATGGCCATGTGGATGGCGCACCCCATCAGACCATCTACAGTGCCCTGATGATCAGATCAGAGGATGCTGGCTTTGTGGTGATTACAGGTGTGATGAGCAGAAGATACCTCTGCATGGATTTCAGAGGCAACATTTTTGGATCACACTATTTCGACCCGGAGAACTGCAGGTTCCAACACCAGACGCTGGAAAACGGGTACGACGTCTACCACTCTCCTCAGTATCACTTCCTGGTCAGTCTGGGCCGGGCGAAGAGAGCCTTCCTGCCAGGCATGAACCCACCCCCGTACTCCCAGTTCCTGTCCCGGAGGAACGAGATCCCCCTAATTCACTTCAACACCCCCATACCACGGCGGCACACCCAGAGCGCCGAGGACGACTCGGAGCGGGACCCCCTGAACGTGCTGAAGCCCCGGGCCCGGATGACCCCGGCCCCGGCCTCCTGTTCACAGGAGCTCCCGAGCGCCGAGGACAACAGCCCGATGGCCAGTGACCCATTAGGGGTGGTCAGGGGCGGTCGAGTGAACACGCACGCTGGGGGAACGGGCCCGGAAGGCTGCCGCCCCTTCGCCAAGTTCATCGGAGGTGGAGGTTCAGCCCCAGAAGCAGCAGGTGGTCCATCAGTTTTTCTTTTCCCTCCCAAACCCAAGGATACGCTGATGATCTCTCGCACGCCTGAGGTGACATGCGTCGTAGTAGACGTGAGCCACGAAGATCCCGAGGTGAAGTTCAATTGGTATGTGGACGGAGTAGAAGTGCATAACGCGAAAACTAAGCCGCGCGAGGAACAATATAACAGTACTTACAGGGTGGTATCCGTGCTCACAGTCCTGCACCAGGACTGGCTGAACGGTAAGGAATACAAGTGCAAAGTAAGCAACAAGGCACTTCCCGCGCCTATTGAGAAAACAATCTCCAAGGCGAAGGGACAACCAAGAGAACCTCAGGTTTACACTCTCCCGCCTTCCAGGGAAGAGATGACCAAAAATCAAGTTTCCCTGACTTGCCTCGTCAAAGGATTCTACCCTTCCGACATTGCTGTTGAATGGGAAAGCAATGGACAACCAGAGAACAACTACAAGACAACACCCCCGGTGCTGGATAGTGACGGATCTTTCTTTCTCTACTCAAAGCTGACCGTGGATAAGTCCAGGTGGCAGCAGGGAAACGTGTTTTCCTGCTCTGTCATGCATGAAGCGCTGCATAATCACTATACCCAGAAGTCTCTGAGCTTGAGCCCAGGCAAGTAA

SEQ ID NO: 49 - protein, 1450 aa, Artificial
  fusion peptide derived from human
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLGSGGGGSGGGGSGGGGSLKYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFIGGGGSAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 1449 aa, Artificial
  fusion peptide derived from human
ATGTTGGGGGCCCGCCTCAGGCTCTGGGTCTGTGCCTTGTGCAGCGTCTGCAGCATGAGCGTCCTCAGAGCCTATCCCAATGCCTCCCCACTGCTCGGCTCCAGCTGGGGTGGCCTGATCCACCTGTACACAGCCACAGCCAGGAACAGCTACCACCTGCAGATCCACAAGAATGGCCATGTGGATGGCGCACCCCATCAGACCATCTACAGTGCCCTGATGATCAGATCAGAGGATGCTGGCTTTGTGGTGATTACAGGTGTGATGAGCAGAAGATACCTCTGCATGGATTTCAGAGGCAACATTTTTGGATCACACTATTTCGACCCGGAGAACTGCAGGTTCCAACACCAGACGCTGGAAAACGGGTACGACGTCTACCACTCTCCTCAGTATCACTTCCTGGTCAGTCTGGGCCGGGCGAAGAGAGCCTTCCTGCCAGGCATGAACCCACCCCCGTACTCCCAGTTCCTGTCCCGGAGGAACGAGATCCCCCTAATTCACTTCAACACCCCCATACCACGGCGGCACACCCAGAGCGCCGAGGACGACTCGGAGCGGGACCCCCTGAACGTGCTGAAGCCCCGGGCCCGGATGACCCCGGCCCCGGCCTCCTGTTCACAGGAGCTCCCGAGCGCCGAGGACAACAGCCCGATGGCCAGTGACCCATTAGGGGTGGTCAGGGGCGGTCGAGTGAACACGCACGCTGGGGGAACGGGCCCGGAAGGCTGCCGCCCCTTCGCCAAGTTCATCGGAGGTGGAGGTTCAAAAACCCACACGTGTCCTCCTTGTCCTGCCCCAGAAGCAGCAGGTGGTCCATCAGTTTTTCTTTTCCCTCCCAAACCCAAGGATACGCTGATGATCTCTCGCACGCCTGAGGTGACATGCGTCGTAGTAGACGTGAGCCACGAAGATCCCGAGGTGAAGTTCAATTGGTATGTGGACGGAGTAGAAGTGCATAACGCGAAAACTAAGCCGCGCGAGGAACAATATAACAGTACTTACAGGGTGGTATCCGTGCTCACAGTCCTGCACCAGGACTGGCTGAACGGTAAGGAATACAAGTGCAAAGTAAGCAACAAGGCACTTCCCGCGCCTATTGAGAAAACAATCTCCAAGGCGAAGGGACAACCAAGAGAACCTCAGGTTTACACTCTCCCGCCTTCCAGGGAAGAGATGACCAAAAATCAAGTTTCCCTGACTTGCCTCGTCAAAGGATTCTACCCTTCCGACATTGCTGTTGAATGGGAAAGCAATGGACAACCAGAGAACAACTACAAGACAACACCCCCGGTGCTGGATAGTGACGGATCTTTCTTTCTCTACTCAAAGCTGACCGTGGATAAGTCCAGGTGGCAGCAGGGAAACGTGTTTTCCTGCTCTGTCATGCATGAAGCGCTGCATAATCACTATACCCAGAAGTCTCTGAGCTTGAGCCCAGGCAAGTAA

SEQ ID NO: 51 - protein, 482 aa, Artificial
  fusion peptide derived from human
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFIGGGGSKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 1422 aa, Artificial
  fusion peptide derived from human
ATGTTGGGGGCCCGCCTCAGGCTCTGGGTCTGTGCCTTGTGCAGCGTCTGCAGCATGAGCGTCCTCAGAGCCTATCCCAATGCCTCCCCACTGCTCGGCTCCAGCTGGGGTGGCCTGATCCACCTGTACACAGCCACAGCCAGGAACAGCTACCACCTGCAGATCCACAAGAATGGCCATGTGGATGGCGCACCCCATCAGACCATCTACAGTGCCCTGATGATCAGATCAGAGGATGCTGGCTTTGTGGTGATTACAGGTGTGATGAGCAGAAGATACCTCTGCATGGATTTCAGAGGCAACATTTTTGGATCACACTATTTCGACCCGGAGAACTGCAGGTTCCAACACCAGACGCTGGAAAACGGGTACGACGTCTACCACTCTCCTCAGTATCACTTCCTGGTCAGTCTGGGCCGGGCGAAGAGAGCCTTCCTGCCAGGCATGAACCCACCCCCGTACTCCCAGTTCCTGTCCCGGAGGAACGAGATCCCCCTAATTCACTTCAACACCCCCATACCACGGCGGCACACCCAGAGCGCCGAGGACGACTCGGAGCGGGACCCCCTGAACGTGCTGAAGCCCCGGGCCCGGATGACCCCGGCCCCGGCCTCCTGTTCACAGGAGCTCCCGAGCGCCGAGGACAACAGCCCGATGGCCAGTGACCCATTAGGGGTGGTCAGGGGCGGTCGAGTGAACACGCACGCTGGGGGAACGGGCCCGGAAGGCTGCCGCCCCTTCGCCAAGTTCATCGGAGGTGGAGGTTCAGCCCCAGAAGCAGCAGGTGGTCCATCAGTTTTTCTTTTCCCTCCCAAACCCAAGGATACGCTGATGATCTCTCGCACGCCTGAGGTGACATGCGTCGTAGTAGACGTGAGCCACGAAGATCCCGAGGTGAAGTTCAATTGGTATGTGGACGGAGTAGAAGTGCATAACGCGAAAACTAAGCCGCGCGAGGAACAATATAACAGTACTTACAGGGTGGTATCCGTGCTCACAGTCCTGCACCAGGACTGGCTGAACGGTAAGGAATACAAGTGCAAAGTAAGCAACAAGGCACTTCCCGCGCCTATTGAGAAAACAATCTCCAAGGCGAAGGGACAACCAAGAGAACCTCAGGTTTACACTCTCCCGCCTTCCAGGGAAGAGATGACCAAAAATCAAGTTTCCCTGACTTGCCTCGTCAAAGGATTCTACCCTTCCGACATTGCTGTTGAATGGGAAAGCAATGGACAACCAGAGAACAACTACAAGACAACACCCCCGGTGCTGGATAGTGACGGATCTTTCTTTCTCTACTCAAAGCTGACCGTGGATAAGTCCAGGTGGCAGCAGGGAAACGTGTTTTCCTGCTCTGTCATGCATGAAGCGCTGCATAATCACTATACCCAGAAGTCTCTGAGCTTGAGCCCAGGCAAGTAA

SEQ ID NO: 53 - protein, 473 aa, Artificial
  fusion peptide derived from human
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTQSAEDDSERDPLNVLKPRARMTPAPASCSQELPSAEDNSPMASDPLGVVRGGRVNTHAGGTGPEGCRPFAKFIGGGGSAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK